(19) United States
(12) Patent Application Publication
(10) Pub. No.: US 2010/0292085 A1
(43) Pub. Date: Nov. 18, 2010

(54) Methods and compositions for characterization of HSD1 inhibitors

(76) Inventors: Pek Yee Lum, Seattle, WA (US); Rolf Thieringer, Highland Park, NJ (US); Serguei B. Stepaniants, Redmond, WA (US); Cheryl LeGrand, Langhome, PA (US); Sarah Dreikorn, Scotch Plains, NJ (US)

Correspondence Address: R. Douglas Bradley;Merck & Co., Inc., Patent Department RY60-30, P.O. Box 2000, Rahway, NJ, 07065-0907, US

(21) Appl. No.: 12/584,317

(22) Filed: Sep. 3, 2009

Related U.S. Application Data
(63) Continuation of application No. 11/595,699, filed on Nov. 9, 2006, now abandoned.
(60) Provisional application No. 60/736,040, filed on Nov. 10, 2005.

(51) Int. Cl. C40B 30/00; C12Q 1/68; G01N 33/574

(52) U.S. Cl. 506/7; 435/6; 435/7.23

(57) ABSTRACT

One aspect of the present invention relates to methods of identifying cortisone response signature gene sets and methods of using the identified gene sets to identify compounds that modulate HSD1 activity. In some embodiments, methods are provided to use cortisone response gene sets to estimate the HSD1 activity. Another aspect of the present inventive relates to methods for identification of off-target signature gene sets that can be used to estimate HSD1 compound induced off-target activity and methods for classification of compounds that modulate HSD1 activity. Another aspect of the present invention relates to cell lines that over-expresses HSD1 and methods of use thereof. Additional embodiments of the of the invention are described in the specification provided herein. The contents of this ABSTRACT are not intended to in anyway limit the scope of the invention claimed herein.

This application is a continuation of application Ser. No. 11/595,699, filed on Nov. 9, 2006, which claims benefit of U.S. Provisional Patent Application Ser. No. 60/736,040 filed on Nov. 10, 2005, both of which are incorporated by reference herein in their entirety.

This application includes a Sequence Listing submitted on compact disc, recorded on two compact discs, including one duplicate, containing Filename RS0225CA.txt, of size 1,433,600 bytes, created Aug. 19, 2009. The Sequence Listing on the compact discs is incorporated by reference herein in its entirety.

### BACKGROUND OF THE INVENTION

The references cited herein are not admitted to be prior art to the claimed invention.

Obesity is associated with a number of adverse health outcomes, such as insulin resistance, dyslipidemia, glucose intolerance, and coronary heart disease. Features of metabolic syndrome appear to be related to adiposity, in particular visceral fat mass (mesenteric and omental fat depots). The anatomical distribution of adipose tissue can be influenced by glucocorticoids. The enzyme 11β-hydroxysteroid dehydrogenase Type I (11β-HSD1 or HSD1) catalyzes the interconversion of inactive glucocorticoid precursors to active glucocorticoids. In addition to affecting fat topology, glucocorticoids also regulate the differentiation of adipose stromal cells and affect the function of adipocytes (Hauner et al., 1989, J. Clin. Invest. 84:1663-1670; Bronnegard et al., 1995, J. Clin. Endocrinol. Metab. 80:3608-3612). Glucocorticoids are also potent regulators of key enzymes involved in hepatic gluconeogenesis, including phosphoenolpyruvate carboxykinase (PEPCK) and glucose-6-phosphatase (G6 Pase) (reviewed in Barthel and Schmoll, 2003, Am. J. Physiol. Endocrinol. Metab. 285:E685-E692). Thus, HSD1 may play an important role in obesity and metabolic syndrome-related outcomes (reviewed in 2004, Stulnig and Waldhäusl, 2004, Diabetologia 47:1-11; Wolf, 2002, Nutrition Rev. 60:148-151).

The importance of glucocorticoids as regulators of glucose and lipid homeostasis in humans is exemplified by Cushing's syndrome. Glucocorticoid excess in Cushing's syndrome leads to insulin resistance, diabetes, dyslipidemia, and redistribution of fat to visceral depots (reviewed in Peeke and Chrousos, 1995, Ann. N.Y. Acad. Sci. 771:665-676). Moreover, use of glucocorticoid therapy as anti-inflammatory or immunosupporessive agents is associated with side effects induced by disturbance of glucose metabolism, such as insulin resistance (reviewed in Schäcke et al., 2002, Pharm. Ther. 96:23-43). Draper et al. (2003, Nat. Genet. 34:434-439) demonstrated that mutations in HSD1 and hexose-6-phosphate dehydrogenase interact to cause cortisone reductase deficiency (absence of cortisone activation), which is characterized by obesity, insulin resistance, infertility, and hirsutism. As in the obese Zucker rat model (Livingstone et al., 2000), tissue specific changes in glucocorticoid metabolism were observed in obese humans, with enhanced HSD1 activity in adipose tissue, which may contribute to obesity (Rask et al., 2001, J. Clin. Endocrinol. Metab. 86:1418-1421). In a study of Pima Indians and Caucasians, higher adipose HSD1 activity was associated with adiposity and insulin resistance (Lindsay et al., 2003, J. Clin. Endocrinol. Metab. 88:2738-2744).

Specific inhibitors of HSD1 have been identified. Arylsulfonamidothiazoles have been shown to be potent and selective inhibitors of HSD1, with no significant activity against HSD2 (Barf et al., 2002, J. Med. Chem. 45:3813-3815). Flavanone has also been identified as an HSD1 specific inhibitor in a rapid screening assay (Schweizer et al., 2003, Mol. Cell. Endocrin. 212:41-49). Carbenoxolone has been found to inhibit hepatic HSD1 reductase activity, resulting in increased hepatic insulin sensitivity (Walker et al., 1995, J. Clin. Endocrinol. Metab. 80:3155-3159). Selective inhibitors of HSD1 useful for the treatment of diabetes, obesity, and other lipid disorders have been disclosed, see for example, U.S. Pat. No. 6,730,690; WO 03/104208, WO 03/104207, and WO 03/065983. WO 04/058741 and WO 04/058730 describe triazole derivatives that are selective inhibitors of HSD1. Amide derivatives have also been described as HSD1 inhibitors (WO04/065351)

HSD1 is being used to develop targeted therapies for the treatment of obesity and metabolic syndrome. Since HSD1 produces cortisol, inhibition of HSD1 by a small molecule should lead to a decrease in cortisol-related responses. Several small molecules have been evaluated in animal models as agents affecting the different risk factors associated with metabolic syndrome.

There is a need for assays that are compatible with current clinical laboratory instrumentation and that are capable of identifying HSD1 compounds and estimating the inhibitory activity of HSD1 compounds. Clinically, it is difficult to predict the in vivo efficacy of HSD1 inhibitors based on their pharmacodynamic effects. As mentioned above, a number of HSD1 inhibitor compounds have been described which show a range of in vivo inhibitor activity that is sometimes discordant with their respective pharmacokinetic/pharmacodynamic profiles. Thus, there is also a need for assays that are capable of monitoring off-target effects of inhibitory HSD1 compounds, allowing better classification of compounds before entering animal efficacy studies.

### BRIEF DESCRIPTION OF SEQUENCE LISTING

This application includes a Sequence Listing submitted on compact disc, recorded on two compact discs, labeled “Copy 1” and “Copy 2” including one duplicate, labeled “Computer Readable Copy”, containing File Name “RS0225.txt”, created on Oct. 20, 2006, consisting of 1,433,600 bytes. The sequence listing on the compact discs is incorporated by reference herein in its entirety.

### DETAILED DESCRIPTION OF THE INVENTION

This section presents a detailed description of the many aspects and embodiments representative of the inventions disclosed herein. This description is by way of several exemplary illustrations, of varying detail and specificity. Other features and advantages of these embodiments are apparent from the additional descriptions provided herein, including the different examples. The provided examples illustrate different components and methodology useful in practicing various embodiments of the invention. The examples do not limit the claimed invention. Based on the present disclosure the ordinary skilled artisan can identify and employ other components and methodology useful for practicing the present invention.

Though enzymatically bidirectional, in vivo HSD1 is believed to function as an oxidoreductase, converting inactive cortisone to active cortisol, thereby regulating the degree of glucocorticoid access to its receptors. HSD1 is a particularly attractive target for regulating gluconeogenesis, insulin sensitivity, and other disorders caused by excess glucocorticoid action.

The cells and methods disclosed and claimed herein are based on the discovery and characterization of gene expression genesets that are specific for: a) cellular response to cortisone as an estimate of HSD1 activity; and b) off-target activity in response to an agent that modulates HSD1 activity. In particular, gene expression profiling data from cultured HepG2 hepatoma cells over-expressing HSD1 were used to identify a set of genes whose expression levels are correlated with response to cortisone treatment and whose gene expression levels are muted by concomitant treatment of an HSD1 inhibitor and cortisone. In one aspect of the invention, the cortisone response geneset is useful to detect HSD1 inhibition in a wide variety of cell samples obtained from in vivo or in vitro sources. In another aspect of the invention, gene expression profiling data from cultured HepG2 cells was used to identify a set of genes that are selectively regulated in response to a specific HSD1 inhibitor and whose expression levels are independent of the presence of cortisone, thus, indicating off-target activity.

In some embodiments of the invention, the cortisone response signature gene sets, which are disclosed and claimed herein, have utility in the context of providing a method for monitoring the inhibition of HSD1 activity via gene expression profiling. Embodiments of the off-target signature gene sets, which are disclosed and claimed herein, have utility in the context of providing a method for detection of off-target activity and for classification of compounds that modulate HSD1 activity. Another aspect of the present invention relates to hepatoma cell lines that over-expresses HSD1 and methods of use thereof.

### I. DEFINITIONS

Unless defined otherwise, all technical and scientific terms used herein have the meaning commonly understood by one of ordinary skill in the art to which this invention belongs. Practitioners are particularly directed to Sambrook et al. (1989) Molecular Cloning: A Laboratory Manual, 2d ed., Cold Spring Harbor Press, Plainsview, N.Y. (1989), and Ausubel et al., Current Protocols in Molecular Biology (Supplement 47), John Wiley & Sons, New York (1999), for definitions and terms of the art.

As used herein, “measuring expression levels,” “obtaining an expression level” and the like, includes methods that quantify a gene expression level of, for example, a transcript of a gene or a protein encoded by a gene, as well as methods that determine whether a gene of interest is expressed at all. Thus, an assay which provides a “yes” or “no” result without necessarily providing quantification, of an amount of expression is an assay that “measures expression” as that phrase is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see for example, U.S. Pat. No. 5,569,588) nuclease protection, RT-PCR, microarray profiling, differential display, 2D gel electrophoresis, SELDI-TOF, ICAT, enzyme assay, antibody assay, and the like.

The term “gene expression”, as used herein, refers to the process of transcription and translation of a gene to produce a gene product, be it RNA or protein. Thus, modulation of gene expression may occur at any one or more of many levels, including transcription, post-transcriptional processing, translation, post-translational modification, and the like.

As used herein, “HSD1” refers to a protein having 11-β-Hydroxysteroid Dehydrogenase Type I activity (HSD1 activity). HSD1 is alternatively known in the art as 11-β-HSD1 or HSD11β1. Exemplary sequences include, for example, the human HSD1 protein, encoded by a nucleic acid molecule, NM_{—}005525, comprising SEQ ID NO: 1. An amino acid sequence for human HSD1, Ref Seq NP_{—}005516, is provided by SEQ ID NO: 2. In addition, “HSD1” is not limited to human HSD1 proteins. The methods exemplified herein may also be used with HSD1 sequences identified in other species, such as, but not limited to, chimpanzee, rat, mouse, and dog. An exemplary chimpanzee HSD1 protein, SEQ ID NO: 3, is encoded by a nucleic acid molecule comprising SEQ ID NO: 4. An exemplary rat HSD1 protein, SEQ ID NO: 5, is encoded by a nucleic acid molecule comprising SEQ ID NO: 6. An exemplary mouse HSD1 protein, SEQ ID NO: 7, is encoded by a nucleic acid molecule comprising SEQ ID NO: 8. An exemplary dog HSD1 protein, SEQ ID NO: 9, is encoded by SEQ ID NO: 10.

As used herein, “HSD1” refers to a polynucleotide encoding HSD1.

The term “HSD1 activity”, as used herein, refers to the oxidoreductase function of HSD1, which converts inactive glucocorticoids (cortisone in humans and 11-dehydrocorticosterone in rats and mice) into their active forms (cortisol and corticosterone, respectively).

The term “HSD1-agent”, as used herein, refers to any substance which alters HSD1 activity. Thus, the HSD1-agent may be a chemical compound, such as a small molecule or complex organic compound, a protein, an antibody, or a genetic construct which acts at the DNA, mRNA, or protein level in a subject. The HSD1-agent may act directly or indirectly, and may modulate the activity of a substance which itself modulates the activity of HSD1.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

The phrases “isolated protein”, “isolated polypeptide”, “isolated peptide” and “isolated oligopeptide” refer to a protein (or respectively to a polypeptide, peptide, or oligopeptide) that is non-identical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

The terms “over-expression”, “over-expresses”, “over-expressing” and the like, refer to the state of altering a subject such that expression of one or more genes in said subject is significantly higher, as determined using one or more statistical tests, than the level of expression of said gene or genes in the same unaltered subject or an analogous unaltered subject.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the percent “sequence identity” of two amino acid sequences is determined using the BLAST family of programs (Altschul et al., 1990, J. Mol. Biol., 215, 403-410; Altschul et al., 1997, Nucleic Acids Res., 25:389-3402, available from the National Center for Biotechnology Information (NCBI), Bethesda, Md., USA and accessible through the home page of the NCBI at ncbi.nlm.nih.gov/BLAST/) using the default settings. Preferably, the BLOSUM62 amino acid substitution matrix (Henikoff and Henikoff, 1992, Proc. Nat. Acad. Sci. USA, 89:10915-10919) is used in polypeptide sequence comparisons including where nucleotide sequences are first translated into amino acid sequences before comparison.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

As used herein, “subject”, as refers to an organism and to cells or tissues derived therefrom, including, for example, cultured cell lines. For example, an organism may be an animal, including but not limited to, an animal, such as a cow, a pig, a mouse, a rat, a chicken, a cat, a dog, etc., and is usually a mammal, such as a human.

### II. ASPECTS AND EMBODIMENTS OF THE INVENTION

### A. HepG2 Cells Over-Expressing HSD1

A first aspect of the invention provides a human hepatoma cell line that has been modified to over-express HSD1. In one embodiment, the HSD1 is expressed from a polynucleotide comprising a sequence encoding human HSD1. An exemplary human HSD1 nucleic acid sequence is SEQ ID NO: 1. In another embodiment, the over expressed HSD1 is encoded by any polynucleotide sequence that encodes SEQ ID NO: 2. In other embodiments, the over-expressed HSD1 comprises a sequence encoding a polypeptide having at least 95% sequence identity to SEQ ID NO: 2. In another embodiment, the HepG2 hepatoma cell line over-expresses HSD1 at least about 2-fold higher, at least about 5-fold higher, or at least about 10-fold higher compared to expression in a non-modified hepatoma cell line. In addition, embodiments of this aspect may be practiced using non-human HSD1. For example, a human hepatoma cell line can be made that over-expresses non-human HSD1 sequences identified in other species, such as, but not limited to, rat HSD1, NP_{—}058776 (SEQ ID NO: 5), mouse HSD1, NP_{—}032314 (SEQ ID NO: 7), chimpanzee HSD1, XP_{—}514165 (SEQ ID NO: 3) or chimeric HSD1 made by combining sequences from one or more species, so long as the overexpressed HSD1 has detectable HSD1 activity in the human hepatoma cell line. Methods for detecting HSD1 activity are known in the art. One such method for detecting HSD1 activity is disclosed in Example 1 herein. Functional variants of the above HSD1 sequences may also be used in the practice of certain aspects of the invention. Functional variants have a detectable level of HSD1 activity. In some embodiments of the invention, functional HSD1 variants have at least 95% sequence identity to one or more of the above listed mammalian HSD1 proteins. In other embodiments of the invention, functional HSD1 variants have at least 90% sequence identity or at least 85% sequence identity to one or more of the above listed mammalian HSD1 proteins.

An exemplary human hepatoma cell line is HepG2 hepatoma cell line (ATCC HB8065) (Knowles et al., 1980, Science 209:497-499). Methods for the preparation and propagation of HepG2 hepatoma cells have been disclosed (see U.S. Pat. No. 4,393,133). In other embodiments of the invention, HSD1 over-expression may be achieved in other human hepatoma cell lines, including but not limited to HuH-7 (Nakabayashi et al., 1982, Cancer Res. 42:3858-3863). HSD1 expression may also be achieved in cell lines from other mammalian species and used with the methods described herein, providing that the cell line also expresses a glucocorticoid receptor, either endogenous or recombinant, capable of responding to the active glucocorticoid product of HSD1, cortisol or corticosterone. An exemplary human glucocorticoid receptor (GCCR or NR3C1) is provided by NP_{—}000167 (SEQ ID NO: 11).

A HepG2 cell line over-expressing HSD1 can be used, for example, in assays to estimate HSD1 activity in response to cortisone, to identify agents that modify the activity of HSD1, or to rank order agents that modify HSD1 activity according to changes in cortisone response signature gene expression levels. Alternatively, the HepG2 cell line can be used in assays to classify HSD1 agents by monitoring HSD1 off-target activity.

A number of approaches to achieve over-expression of HSD1 in hepatoma cells can be used. In some embodiments, expression is achieved in a host cell using an expression vector comprising a polynucleotide encoding active HSD1. An exemplary expression vector contains, for example, a recombinant nucleic acid encoding an HSD1 polypeptide operably linked with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. In most embodiments, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids, viruses or transposons.

In most embodiments, the promoter element is typically selected from promoters that are functional in mammalian cells, although prokaryotic promoters and promoters functional in other eukaryotic cells can be used. The promoter is typically selected from promoter sequences of viral or eukaryotic genes. The promoter can be a promoter that functions in a ubiquitous manner, e.g., are functional in a large number of cell types, such as, for example, promoters of α-actin or β-actin or, alternatively, a tissue-specific manner. In some embodiments of the invention, tissue-specific promoters functional in hepatocytes may be used. In other embodiments promoters are selected that respond to specific stimuli, such as, promoters that bind steroid hormone receptors. In still other embodiments viral promoters may be used, for example, the Rous sarcoma virus (RSV) LTR promoter. Other examples of useful promoters are inducible promoters can be used to modulate, e.g., turn on and off or otherwise change the level of expression of HSD1 during the life-time of the cells in which the HSD1 over-expressing vector construct is present. Chimeric promoters comprising sequence elements from two or more different promoters may also be used. Additionally, any of these promoters, or the endogenous HSD1 promoter in the host cell, can be modified by additional of regulatory sequences, such as enhancer sequences.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacI (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460).

A number of viral-based expression systems can be used to express HSD1 polypeptides in mammalian host cells. For example, if an adenovirus is used as an expression vector, sequences encoding HSD1 polypeptides can be ligated into an adenovirus transcription/translation complex comprising the late promoter and tripartite leader sequence. Insertion in a non-essential E1 or E3 region of the viral genome can be used to obtain a viable virus which is capable of expressing a HSD1 polypeptide in infected host cells (Logan and Shenk, 1984, Proc. Natl. Acad. Sci. 81:3655-3659). If desired, transcription enhancers, such as the Rous sarcoma virus (RSV) enhancer, can be used to increase expression in mammalian host cells.

Alternatively, the expression vector comprises a transposable element that contains an HSD1 gene that when transposed into a target cell chromosome is expressed thereby over-producing HSD1. Exemplary transposons include Sleeping Beauty (Essner et al., 2005 Curr. Opin. Pharmacol. 5:513-519) and piggyBac (Ding et al., 2005, Cell 122:473-483), as well as any other transposable elements that are functional in human cells.

Human artificial chromosomes (HACs) also can be used to deliver larger fragments of DNA than can be contained and expressed in a plasmid. HACs of 6 to 10 megabases (Mb) are constructed and delivered to cells via conventional delivery methods (e.g., liposomes, polycationic amino polymers, or vesicles).

In still other embodiments, an endogenous HSD1 gene that is normally transcriptionally inactive in HepG2 cells is activated by recombination of an exogenously engineered polynucleotide containing a transcription promoter that is active in HepG2 cells. Exemplary methods and vectors for recombination based endogenous gene activation are described in U.S. Pat. No. 5,641,670; U.S. Pat. No. 5,733,761; U.S. Pat. No. 6,187,305; U.S. Pat. No. 6,623,958; and U.S. Pat. No. 6,576,443.

In some embodiments of the invention, long-term, high yield intracellular expression of HSD1 protein may be desired. Stable expression of HSD1 in cell lines can be achieved using expression vectors which contain an origin of DNA replication functional in the target cell, such as for example a viral origin of DNA replication, and/or endogenous expression elements and a selectable marker gene on the same or on a separate vector. Alternatively, stable expression may be obtained via recombination of an exogenously provided HSD1 gene as described previously. The selectable marker allows growth and recovery of cells which successfully express the introduced HSD1 enzyme sequences. Resistant clones of stable transformed cells can be proliferated using tissue culture techniques appropriate to the cell type. See for example, ANIMAL CELL CULTURE, R. I. Freshney, ed., 1986. Selection systems include, but are not limited to herpes simplex virus thymidine kinase, adenine phosphoribosyltransferase, antibiotic resistance, antimetabolite resistance, and visible markers such as luciferase.

Lipsomal administration of a modulator that increases HSD1 expression, such as C/EBPα (Williams et al., 2000, J. Biol. Chem. 275:30232-30239), may also be used to stimulate HSD1 expression. Alternatively a host cell can be transformed or transfected with an expression vector encoding an HSD1 modulator to achieve increased expression of endogenous or exogenous (encoded in a vector) HSD1, providing the appropriate binding sites for the modulator are not excluded in the vector encoded HSD1 gene.

Recombinant DNA molecules that feature precise fusions of polynucleotide sequences can also be assembled using standard recombinational subcloning techniques. Recombination-mediated, PCR-directed, or PCR-independent plasmid construction in yeast is well known in the art (see Hua et al., 1997, Plasmid 38:91-96; Hudson et al., 1997, Genome Res. 7:1169-1173; Oldenburg et al., 1997, Nucleic Acids Res. 25:451-452; Raymond et al., 1999, BioTechniques 26:134-8, 140-1). Overlapping sequences between the donor DNA fragments and the acceptor plasmid permit recombination in yeast. An example of recombination-mediated plasmid construction in Saccharomyces cerevisiae is described in Oldenburg et al. (1997, Nucleic Acids Res. 25:451-452): a DNA segment of interest was amplified by PCR so that the PCR product had 20-40 by of homology at each end to the region of the plasmid at which recombination was to occur. The PCR product and linearized plasmid were co-transformed into yeast, and recombination resulted in replacement of the region between the homologous sequences on the plasmid with the region carried by the PCR fragment. The recombinational method of plasmid construction bypasses the need for extensive modification and ligation steps and does not rely on available restriction sites. These cloning vectors can then be utilized for protein expression in multiple systems, including, for example, HepG2 hepatoma cell lines.

In another embodiment of the invention, nucleic acid sequences encoding HSD1 may be ligated to a heterologous sequence resulting in translation of a fusion protein in any of the aforementioned host systems. For example, a chimeric HSD1 protein containing a heterologous moiety that can be recognized by a commercially available antibody may facilitate detection and quantification of HSD1 expression in the host system. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the HSD1 encoding sequence and the heterologous protein sequence, so that HSD1 may be cleaved away from the heterologous moiety following purification. Methods for fusion protein expression and purification are discussed in Ausubel (Current Protocols in Molecular Biology, John Wiley, 1987-1998). A variety of commercially available kits may also be used to facilitate expression and purification of fusion proteins.

To enhance expression in a particular host it may be useful to modify the HSD1 coding sequence to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Introduction of mRNA into cell based systems can be achieved, for example, by microinjection or electroporation. A variety of methods are well known in the art for manipulating the expression of a gene of interest. It is to be understood that methods other than those exemplified herein may be used to manipulate hepatoma cell lines so that they express HSD1 activity.

### III. DETECTING GENE EXPRESSION BY MEASURING GENE TRANSCRIPT EXPRESSION

In some aspects of the present invention, gene expression levels are measured in cells of a cell type that have been contacted with an agent. Gene expression may be measured, for example, by extracting (and optionally purifying) mRNA from the living thing, and using the mRNA as a template to synthesize cDNA which is then labeled (e.g., with a fluorescent dye) and can be used to measure gene expression. While the following exemplary description is directed to embodiments of the invention in which the extracted mRNA is used as a template to synthesize cDNA, which is then labeled, it will be understood that the extracted mRNA can also be used as a template to synthesize cRNA which can then be labeled and can be used to measure gene expression.

RNA molecules useful as templates for cDNA synthesis can be isolated from any organism or part thereof, including organs, tissues, and/or individual cells. Any suitable RNA preparation can be utilized, such as total cellular RNA, or such as cytoplasmic RNA or such as an RNA preparation that is enriched for messenger RNA (mRNA), such as RNA preparations that include greater than 70%, or greater than 80%, or greater than 90%, or greater than 95%, or greater than 99% messenger RNA. Typically, RNA preparations that are enriched for messenger RNA are utilized to provide the RNA template in the practice of the methods of this aspect of the invention. Messenger RNA can be purified in accordance with any art-recognized method, such as by the use of oligo-dT columns (see, e.g., Sambrook et al., 1989, Molecular Cloning—A Laboratory Manual (2nd Ed.), Vol. 1, Chapter 7, Cold Spring Harbor Laboratory, Cold Spring Harbor, N.Y.).

Total RNA may be isolated from cells by procedures that involve breaking open the cells and, typically, denaturation of the proteins contained therein. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells using guanidinium thiocyanate lysis followed by CsCl centrifugation to separate the RNA from DNA (Chirgwin et al., 1979, Biochemistry 18:5294-5299). Messenger RNA may be selected with oligo-dT cellulose (see Sambrook et al., supra). Separation of RNA from DNA can also be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

The sample of total RNA typically includes a multiplicity of different mRNA molecules, each different mRNA molecule having a different nucleotide sequence (although there may be multiple copies of the same mRNA molecule). In a specific embodiment, the mRNA molecules in the RNA sample comprise at least 100 different nucleotide sequences. In other embodiments, the mRNA molecules of the RNA sample comprise at least 500, 1,000, 5,000, 10,000, 20,000, 30,000, 40,000, 50,000, 60,000, 70,000, 80,000, 90,000 or 100,000 different nucleotide sequences. In another specific embodiment, the RNA sample is a mammalian RNA sample, the mRNA molecules of the mammalian RNA sample comprising about 20,000 to 30,000 different nucleotide sequences, or comprising substantially all of the different mRNA sequences that are expressed in the cell(s) from which the mRNA was extracted.

In the context of the present example, cDNA molecules are synthesized that are complementary to the RNA template molecules. Each cDNA molecule is preferably sufficiently long (e.g., at least 50 nucleotides in length) to subsequently serve as a specific probe for the mRNA template from which it was synthesized, or to serve as a specific probe for a DNA sequence that is identical to the sequence of the mRNA template from which the cDNA molecule was synthesized. Individual DNA molecules can be complementary to a whole RNA template molecule, or to a portion thereof. Thus, a population of cDNA molecules is synthesized that includes individual DNA molecules that are each complementary to all, or to a portion, of a template RNA molecule. Typically, at least a portion of the complementary sequence of at least 95% (more typically at least 99%) of the template RNA molecules are represented in the population of cDNA molecules.

Any reverse transcriptase molecule can be utilized to synthesize the cDNA molecules, such as reverse transcriptase molecules derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT). A reverse transcriptase lacking RNaseH activity (e.g., SUPERSCRIPT II™ sold by Stratagene, La Jolla, Calif.) has the advantage that, in the absence of an RNaseH activity, synthesis of second strand cDNA molecules does not occur during synthesis of first strand cDNA molecules. The reverse transcriptase molecule should also preferably be thermostable so that the cDNA synthesis reaction can be conducted at as high a temperature as possible, while still permitting hybridization of any required primer(s) to the RNA template molecules.

The synthesis of the cDNA molecules can be primed using any suitable primer, typically an oligonucleotide in the range of ten to 60 bases in length. Oligonucleotides that are useful for priming the synthesis of the cDNA molecules can hybridize to any portion of the RNA template molecules, including the oligo-dT tail. In some embodiments, the synthesis of the cDNA molecules is primed using a mixture of primers, such as a mixture of primers having random nucleotide sequences. Typically, for oligonucleotide molecules less than 100 bases in length, hybridization conditions are 5° C. to 10° C. below the homoduplex melting temperature (Tm); see generally, Sambrook et al. Molecular Cloning: A Laboratory Manual, 2nd ed., Cold Spring Harbor Press, 1987; Ausubel et al., Current Protocols in Molecular Biology, Greene Publishing, 1987).

A primer for priming cDNA synthesis can be prepared by any suitable method, such as phosphotriester and phosphodiester methods of synthesis, or automated embodiments thereof. It is also possible to use a primer that has been isolated from a biological source, such as a restriction endonuclease digest. An oligonucleotide primer can be DNA, RNA, chimeric mixtures or derivatives or modified versions thereof, so long as it is still capable of priming the desired reaction. The oligonucleotide primer can be modified at the base moiety, sugar moiety, or phosphate backbone, and may include other appending groups or labels, so long as it is still capable of priming cDNA synthesis.

An oligonucleotide primer for priming cDNA synthesis can be derived by cleavage of a larger nucleic acid fragment using non-specific nucleic acid cleaving chemicals or enzymes or site-specific restriction endonucleases; or by synthesis by standard methods known in the art, e.g., by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (1988, Nucl. Acids Res. 16:3209-3221), methylphosphonate oligonucleotides can be prepared by use of controlled pore glass polymer supports (Sarin et al., 1988, Proc. Natl. Acad. Sci. U.S.A. 85:7448-7451).

Once the desired oligonucleotide is synthesized, it is cleaved from the solid support on which it was synthesized and treated, by methods known in the art, to remove any protecting groups present. The oligonucleotide may then be purified by any method known in the art, including extraction and gel purification. The concentration and purity of the oligonucleotide may be determined, for example, by examining the oligonucleotide that has been separated on an acrylamide gel, or by measuring the optical density at 260 nm in a spectrophotometer.

After cDNA synthesis is complete, the RNA template molecules can be hydrolyzed, and all, or substantially all (typically more than 99%), of the primers can be removed. Hydrolysis of the RNA template can be achieved, for example, by alkalinization of the solution containing the RNA template (e.g., by addition of an aliquot of a concentrated sodium hydroxide solution). The primers can be removed, for example, by applying the solution containing the RNA template molecules, cDNA molecules, and the primers, to a column that separates nucleic acid molecules on the basis of size. The purified, cDNA molecules, can then, for example, be precipitated and re-dissolved in a suitable buffer.

The cDNA molecules are typically labeled to facilitate the detection of the cDNA molecules when they are used as a probe in a hybridization experiment, such as a probe used to screen a DNA microarray, to identify an efficacy-related population of genes. The cDNA molecules can be labeled with any useful label, such as a radioactive atom (e.g., ³²P), but typically the cDNA molecules are labeled with a dye. Examples of suitable dyes include fluorophores and chemiluminescers.

By way of example, cDNA molecules can be coupled to dye molecules via aminoallyl linkages by incorporating allylamine-derived nucleotides (e.g., allylamine-dATP, allylamine-dCTP, allylamine-dGTP, and/or allylamine-dTTP) into the cDNA molecules during synthesis of the cDNA molecules. The allylamine-derived nucleotide(s) can then be coupled, via an aminoallyl linkage, to N-hydroxysuccinimide ester derivatives (NHS derivatives) of dyes (e.g., Cy-NHS, Cy3-NHS and/or Cy5-NHS). Again by way of example, in another embodiment, dye-labeled nucleotides may be incorporated into the cDNA molecules during synthesis of the cDNA molecules, which labels the cDNA molecules directly.

It is also possible to include a spacer (usually 5-16 carbon atoms long) between the dye and the nucleotide, which may improve enzymatic incorporation of the modified nucleotides during synthesis of the cDNA molecules.

In the context of the present example, the labeled cDNA is hybridized to a DNA array that includes hundreds, or thousands, of identified nucleic acid molecules (e.g., cDNA molecules) that correspond to genes that are expressed in the type of cells wherein gene expression is being analyzed. Typically, hybridization conditions used to hybridize the labeled cDNA to a DNA array are no more than 25° C. to 30° C. (for example, 10° C.) below the melting temperature (Tm) of the native duplex of the cDNA that has the lowest melting temperature (see generally, Sambrook et al. Molecular Cloning: A Laboratory Manual, 2nd ed., Cold Spring Harbor Press, 1987; Ausubel et al., Current Protocols in Molecular Biology, Greene Publishing, 1987). Tm for nucleic acid molecules greater than about 100 bases can be calculated by the formula Tm=81.5+0.41% (G+C)−log(Na+). For oligonucleotide molecules less than 100 bases in length, exemplary hybridization conditions are 5° to 10° C. below Tm.

A. Preparation of microarrays. Nucleic acid molecules can be immobilized on a solid substrate by any art-recognized means. For example, nucleic acid molecules (such as DNA or RNA molecules) can be immobilized to nitrocellulose, or to a synthetic membrane capable of binding nucleic acid molecules, or to a nucleic acid microarray, such as a DNA microarray. A DNA microarray, or chip, is a microscopic array of DNA fragments, such as synthetic oligonucleotides, disposed in a defined pattern on a solid support, wherein they are amenable to analysis by standard hybridization methods (see, Schena, BioEssays 18:427, 1996).

The DNA in a microarray may be derived, for example, from genomic or cDNA libraries, from fully sequenced clones, or from partially sequenced cDNAs known as expressed sequence tags (ESTs). Methods for obtaining such DNA molecules are generally known in the art (see, e.g., Ausubel et al. (eds.), 1994, Current Protocols in Molecular Biology, Vol. 2, Current Protocols Publishing, New York). Again by way of example, oligonucleotides may be synthesized by conventional methods, such as the methods described herein.

Microarrays can be made in a number of ways, of which several are described below. However produced, microarrays preferably share certain characteristics. The arrays are preferably reproducible, allowing multiple copies of a given array to be produced and easily compared with each other. Preferably the microarrays are small, usually smaller than 5 cm², and they are made from materials that are stable under nucleic acid hybridization conditions. A given binding site or unique set of binding sites in the microarray should specifically bind the product of a single gene (or a nucleic acid molecule that represents the product of a single gene, such as a cDNA molecule that is complementary to all, or to part, of an mRNA molecule). Although there may be more than one physical binding site (hereinafter “site”) per specific gene product, for the sake of clarity the discussion below will assume that there is a single site.

In one embodiment, the microarray is an array of polynucleotide probes, the array comprising a support with at least one surface and typically at least 100 different polynucleotide probes, each different polynucleotide probe comprising a different nucleotide sequence and being attached to the surface of the support in a different location on the surface. For example, the nucleotide sequence of each of the different polynucleotide probes can be in the range of 40 to 80 nucleotides in length. For example, the nucleotide sequence of each of the different polynucleotide probes can be in the range of 50 to 70 nucleotides in length. For example, the nucleotide sequence of each of the different polynucleotide probes can be in the range of 50 to 60 nucleotides in length. In specific embodiments, the array comprises polynucleotide probes of at least 2,000, 4,000, 10,000, 15,000, 20,000, 50,000, 80,000, or 100,000 different nucleotide sequences.

Thus, the array can include polynucleotide probes for most, or all, genes expressed in a cell, tissue, organ or organism. In a specific embodiment, the cell or organism is a mammalian cell or organism. In another specific embodiment, the cell or organism is a human cell or organism. In specific embodiments, the nucleotide sequences of the different polynucleotide probes of the array are specific for at least 50%, at least 75%, at least 80%, at least 85%, at least 90%, at least 95%, or at least 99% of the genes in the genome of the cell or organism. Most preferably, the nucleotide sequences of the different polynucleotide probes of the array are specific for all of the genes in the genome of the cell or organism. In specific embodiments, the polynucleotide probes of the array hybridize specifically and distinguishably to at least 10,000, to at least 20,000, to at least 50,000, to at least 80,000, or to at least 100,000 different polynucleotide sequences. In other specific embodiments, the polynucleotide probes of the array hybridize specifically and distinguishably to at least 90%, at least 95%, or at least 99% of the genes or gene transcripts of the genome of a cell or organism. Most preferably, the polynucleotide probes of the array hybridize specifically and distinguishably to the genes or gene transcripts of the entire genome of a cell or organism.

In specific embodiments, the array has at least 100, at least 250, at least 1,000, or at least 2,500 probes per 1 cm², preferably all or at least 25% or 50% of which are different from each other. In another embodiment, the array is a positionally addressable array (in that the sequence of the polynucleotide probe at each position is known). In another embodiment, the nucleotide sequence of each polynucleotide probe in the array is a DNA sequence. In another embodiment, the DNA sequence is a single-stranded DNA sequence. The DNA sequence may be, e.g., a cDNA sequence, or a synthetic sequence.

When a cDNA molecule that corresponds to an mRNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) DNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In some embodiments, cDNA molecule populations prepared from RNA from two different cell populations, or tissues, or organs, or whole organisms, are hybridized to the binding sites of the array. A single array can be used to simultaneously screen more than one cDNA sample. For example, in the context of the present invention, a single array can be used to simultaneously screen a cDNA sample prepared from a living thing that has been contacted with an agent (e.g., candidate HSD1 inhibitor agent), and the same type of living thing that has not been contacted with the agent. The cDNA molecules in the two samples are differently labeled so that they can be distinguished. In one embodiment, for example, cDNA molecules from a cell population treated with a drug is synthesized using a fluorescein-labeled NTP, and cDNA molecules from a control cell population, not treated with the drug, is synthesized using a rhodamine-labeled NTP. When the two populations of cDNA molecules are mixed and hybridized to the DNA array, the relative intensity of signal from each population of cDNA molecules is determined for each site on the array, and any relative difference in abundance of a particular mRNA detected.

In this representative example, the cDNA molecule population from the drug-treated cells will fluoresce green when the fluorophore is stimulated, and the cDNA molecule population from the untreated cells will fluoresce red. As a result, when the drug treatment has no effect, either directly or indirectly, on the relative abundance of a particular mRNA in a cell, the mRNA will be equally prevalent in treated and untreated cells and red-labeled and green-labeled cDNA molecules will be equally prevalent. When hybridized to the DNA array, the binding site(s) for that species of RNA will emit wavelengths characteristic of both fluorophores (and appear brown in combination). In contrast, when the drug-exposed cell is treated with a drug that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, e.g., in Schena et al., 1995, Science 270:467-470, which is incorporated by reference in its entirety for all purposes. An advantage of using cDNA molecules labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA molecules from a single cell, and compare, for example, the absolute amount of a particular mRNA in, e.g., a drug-treated or an untreated cell.

Exemplary microarrays and methods for their manufacture and use are set forth in Hughes et al., 2001, Nature Biotechnology 19:342-347.

B. Preparation of nucleic acid molecules for immobilization on microarrays. As noted above, the “binding site” to which a particular, cognate, nucleic acid molecule specifically hybridizes is usually a nucleic acid, or nucleic acid analogue, attached at that binding site. In one embodiment, the binding sites of the microarray are DNA polynucleotides corresponding to at least a portion of some or all genes in an organism's genome. These DNAs can be obtained by, for example, polymerase chain reaction (PCR) amplification of gene segments from genomic DNA, cDNA (e.g., by reverse transcription or RT-PCR), or cloned sequences. Nucleic acid amplification primers are chosen, based on the known sequence of the genes or cDNA, that result in amplification of unique fragments (i.e., fragments that typically do not share more than 10 bases of contiguous identical sequence with any other fragment on the microarray). Computer programs are useful in the design of primers with the required specificity and optimal amplification properties. See, e.g., Oligo version 5.0 (National Biosciences). Typically each gene fragment on the microarray will be between about 50 by and about 2000 bp, more typically between about 100 by and about 1000 bp, and usually between about 300 by and about 800 by in length.

Nucleic acid amplification methods are well known and are described, for example, in Innis et al., eds., 1990, PCR Protocols: A Guide to Methods and Applications, Academic Press Inc., San Diego, Calif., which is incorporated by reference in its entirety for all purposes. Computer controlled robotic systems are useful for isolating and amplifying nucleic acids.

An alternative means for generating the nucleic acid molecules for the microarray is by synthesis of synthetic polynucleotides or oligonucleotides, e.g., using N-phosphonate or phosphoramidite chemistries (e.g., Froehler et al., 1986, Nucleic Acid Res 14:5399-5407). Synthetic sequences are typically between about 15 and about 100 bases in length, such as between about 20 and about 50 bases.

In some embodiments, synthetic nucleic acids include non-natural bases, e.g., inosine. Where the particular base in a given sequence is unknown or is polymorphic, a universal base, such as inosine or 5-nitroindole, may be substituted. Additionally, it is possible to vary the charge on the phosphate backbone of the oligonucleotide, for example, by thiolation or methylation, or even to use a peptide rather than a phosphate backbone. The making of such modifications is within the skill of one trained in the art.

As noted above, nucleic acid analogues may be used as binding sites for hybridization. An example of a suitable nucleic acid analogue is peptide nucleic acid (see, e.g., Egholm et al., 1993, Nature 365:566-568; see also U.S. Pat. No. 5,539,083).

In another embodiment, the binding (hybridization) sites are made from plasmid or phage clones of genes, cDNAs (e.g., expressed sequence tags), or inserts therefrom (Nguyen et al., 1995, Genomics 29:207-209). In yet another embodiment, the polynucleotide of the binding sites is RNA.

C. Attaching nucleic acids to the solid support. The nucleic acids, or analogues, are attached to a solid support, which may be made, for example, from glass, silicon, plastic (e.g., polypropylene, nylon, polyester), polyacrylamide, nitrocellulose, cellulose acetate or other materials. In general, non-porous supports, and glass in particular, are preferred. The solid support may also be treated in such a way as to enhance binding of oligonucleotides thereto, or to reduce non-specific binding of unwanted substances thereto. For example, a glass support may be treated with polylysine or silane to facilitate attachment of oligonucleotides to the slide.

Methods of immobilizing DNA on the solid support may include direct touch, micropipetting (see, e.g., Yershov et al., 1996, Proc. Natl. Acad. Sci. USA 93:4913-4918), or the use of controlled electric fields to direct a given oligonucleotide to a specific spot in the array. Oligonucleotides are typically immobilized at a density of 100 to 10,000 oligonucleotides per cm², such as at a density of about 1000 oligonucleotides per cm².

A method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., 1996, Proc. Natl. Acad. Sci. USA 93:10614-19.)

In an alternative to immobilizing pre-fabricated oligonucleotides onto a solid support, it is possible to synthesize oligonucleotides directly on the support (see, e.g., Maskos et al., 1993, Nucl. Acids Res. 21:2269-70; Lipshutz et al., 1999, Nat. Genet. 21(1 Suppl):20-4). Methods of synthesizing oligonucleotides directly on a solid support include photolithography (see McGall et al., 1996, Proc. Natl. Acad. Sci. USA 93:13555-60) and piezoelectric printing (Lipshutz et al., 1999, Nat. Genet. 21(1 Suppl):20-4).

A high-density oligonucleotide array may be employed. Techniques are known for producing arrays containing thousands of oligonucleotides complementary to defined sequences, at defined locations on a surface using photolithographic techniques for synthesis in situ (see Pease et al., 1994, Proc. Natl. Acad. Sci. USA 91:5022-5026; Lockhart et al., 1996, Nature Biotechnol. 14:1675-80) or other methods for rapid synthesis and deposition of defined oligonucleotides (Lipshutz et al., 1999, Nat. Genet. 21(1 Suppl):20-4).

In some embodiments, microarrays are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioeletronics 11:687-690; Blanchard, 1998, in Synthetic DNA Arrays in Genetic Engineering, Vol. 20, J. K. Setlow (ed.), Plenum Press, New York at pages 111-123; U.S. Pat. No. 6,028,189 to Blanchard. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes).

Other methods for making microarrays, e.g., by masking (Maskos and Southern, 1992, Nuc. Acids Res. 20:1679-1684), may also be used. In principle, any type of array, for example dot blots on a nylon hybridization membrane (see Sambrook et al., 1989, Molecular Cloning—A Laboratory Manual (2nd ed.), Vols. 1-3, Cold Spring Harbor Laboratory, Cold Spring Harbor, N.Y.), could be used, although, as will be recognized by those of skill in the art, very small arrays are typically preferred because hybridization volumes will be smaller.

D. Signal detection and data analysis. When fluorescently labeled probes are used, the fluorescence emissions at each site of an array can be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645, which is incorporated by reference in its entirety for all purposes). In one embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Shalon et al., 1996, Genome Res. 6:639-645 and in other references cited herein. Alternatively, the fiber-optic bundle described by Ferguson et al., 1996, Nature Biotechnol. 14:1681-1684, may be used to monitor mRNA abundance levels at a large number of sites simultaneously.

Signals are recorded and may be analyzed by computer, e.g., using a 12 bit analog to digital board. In some embodiments the scanned image is de-speckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration.

The relative abundance of an mRNA in two biological samples is scored as a perturbation and its magnitude determined (i.e., the abundance is different in the two sources of mRNA tested), or as not perturbed (i.e., the relative abundance is the same). Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

By way of example, two samples, each labeled with a different fluor, are hybridized simultaneously to permit differential expression measurements. If neither sample hybridizes to a given spot in the array, no fluorescence will be seen. If only one hybridizes to a given spot, the color of the resulting fluorescence will correspond to that of the fluor used to label the hybridizing sample (for example, green if the sample was labeled with Cy3, or red, if the sample was labeled with Cy5). If both samples hybridize to the same spot, an intermediate color is produced (for example, yellow if the samples were labeled with fluorescein and rhodamine). Then, applying methods of pattern recognition and data analysis known in the art, it is possible to quantify differences in gene expression between the samples. Methods of pattern recognition and data analysis are described in e.g., International Publication WO 00/24936, which is incorporated by reference herein.

### IV. DETECTING GENE EXPRESSION BY MEASURING EXPRESSION OF A POPULATION OF PROTEINS

The magnitude of expression of a plurality of genes can be measured, for example, by measuring the magnitude of expression of proteins encoded by the genes.

Any useful method for measuring protein expression patterns can be used. Typically all, or substantially all, proteins are extracted from a living thing, or a portion thereof, e.g., cells. The living thing is typically treated to disrupt cells, for example by homogenizing the cellular material in a blender, or by grinding (in the presence of acid-washed, siliconized, sand if desired) the cellular material with a mortar and pestle, or by subjecting the cellular material to osmotic stress that lyses the cells. Cell disruption may be carried out in the presence of a buffer that maintains the released contents of the disrupted cells at a desired pH, such as the physiological pH of the cells. The buffer may optionally contain inhibitors of endogenous proteases. Physical disruption of the cells can be conducted in the presence of chemical agents (e.g., detergents) that promote the release of proteins.

The cellular material may be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium.

It may be desirable to include one or more protease inhibitors in the protein extraction buffer. Representative examples of protease inhibitors include: serine protease inhibitors (such as phenylmethylsulfonyl fluoride (PMSF), benzamide, benzamidine HCl, ε-Amino-n-caproic acid and aprotinin (Trasylol)); cysteine protease inhibitors, such as sodium p-hydroxymercuribenzoate; competitive protease inhibitors, such as antipain and leupeptin; covalent protease inhibitors, such as iodoacetate and N-ethylmaleimide; aspartate (acidic) protease inhibitors, such as pepstatin and diazoacetylnorleucine methyl ester (DAN); metalloprotease inhibitors, such as EGTA [ethylene glycol bis(β-aminoethyl ether) N,N,N′,N′-tetraacetic acid], and the chelator 1,10-phenanthroline.

The mixture of released proteins may, or may not, be treated to completely or partially purify some of the proteins for further analysis, and/or to remove non-protein contaminants (e.g., carbohydrates and lipids). In some embodiments, the complete mixture of released proteins is analyzed to determine the amount and/or identity of some or all of the proteins. For example, the protein mixture may be applied to a substrate bearing antibody molecules that specifically bind to one or more proteins in the mixture. The unbound proteins are removed (e.g., washed away with a buffer solution), and the amount of bound protein(s) is measured. Representative techniques for measuring the amount of protein using antibodies are described in Harlow and Lane, 1988, Antibodies: A Laboratory Manual, Cold Spring Harbor, N.Y., and include such techniques as the ELISA assay. Moreover, protein microarrays can be used to simultaneously measure the amount of a multiplicity of proteins. A surface of the microarray bears protein binding agents, such as monoclonal antibodies specific to a plurality of protein species. Preferably, antibodies are present for a substantial fraction of the encoded proteins, or at least for those proteins whose amount is to be measured. Methods for making monoclonal antibodies are well known (see, e.g., Harlow and Lane, 1988, Antibodies: A Laboratory Manual, Cold Spring Harbor, N.Y.). Protein binding agents are not restricted to monoclonal antibodies, and can be, for example, scFv/Fab diabodies, affibodies, and aptamers. Protein microarrays are generally described by Templin et al., 2002, Trends Biotechnol. 20:160-166. Representative examples of protein microarrays are described by Zhu et al., 2001, Science 293:2102-2105; and MacBeath and Schreiber, 2000, Science 289:1760-1763.

In some embodiments, the released protein is treated to completely or partially purify some of the proteins for further analysis, and/or to remove non-protein contaminants. Any useful purification technique, or combination of techniques, can be used. For example, a solution containing extracted proteins can be treated to selectively precipitate certain proteins, such as by dissolving ammonium sulfate in the solution, or by adding trichloroacetic acid. The precipitated material can be separated from the un-precipitated material, for example by centrifugation, or by filtration. The precipitated material can be further fractionated if so desired.

By way of example, a number of different neutral or slightly acidic salts have been used to solubilize, precipitate, or fractionate proteins in a differential manner. These include NaCl, Na₂SO₄, MgSO₄ and NH₄(SO₄)₂. Ammonium sulfate is a commonly used precipitant for salting proteins out of solution. The solution to be treated with ammonium sulfate may first be clarified by centrifugation. The solution should be in a buffer at neutral pH unless there is a reason to conduct the precipitation at another pH; in most cases the buffer will have ionic strength close to physiological. Precipitation is usually performed at 0-4° C. (to reduce the rate of proteolysis caused by proteases in the solution), and all solutions should be pre-cooled to that temperature range.

Representative examples of other art-recognized techniques for purifying, or partially purifying, proteins from a living thing are exclusion chromatography, ion-exchange chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or octyl nonpolar groups. The mobile phase is usually an aqueous salt solution. In reversed phase chromatography the matrix is silica that has been substituted with longer n-alkyl chains, usually C₈ (octylsilyl) or C₁₈ (octadecylsilyl). The matrix is less polar than the mobile phase. The mobile phase is usually a mixture of water and a less polar organic modifier.

Separations on hydrophobic interaction chromatography matrices are usually done in aqueous salt solutions, which generally are non-denaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of protein purification, hydrophobic interaction chromatography depends on surface hydrophobic groups and is usually carried out under conditions which maintain the integrity of the protein molecule. Reversed-phase chromatography depends on the native hydrophobicity of the protein and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by chemical bonding to the stationary phase. These fixed charges carry a counter-ion of opposite sign. This counter-ion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the non-fixed ions by the molecule. Elution, in turn, involves displacement of the molecule from the fixed charges by a new counter-ion with a greater affinity for the fixed charges than the molecule, and which then becomes the new, non-fixed ion.

The ability of counter-ions (salts) to displace molecules bound to fixed charges is a function of the difference in affinities between the fixed charges and the non-fixed charges of both the molecule and the salt. Affinities in turn are affected by several variables, including the magnitude of the net charge of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding proteins at pH values above their pI. The polystyrene matrix can be obtained with quaternary ammonium functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as proteins) is applied in a discrete volume or zone at the top of a gel filtration column and allowed to percolate through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

A technique that can be applied to the purification of proteins is High Performance Liquid Chromatography (HPLC). HPLC is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance adsorption-desorption kinetics and diminish band spreading, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography.

An exemplary technique that is useful for measuring the amounts of individual proteins in a mixture of proteins is two dimensional gel electrophoresis. This technique typically involves isoelectric focusing of a protein mixture along a first dimension, followed by SDS-PAGE of the focused proteins along a second dimension (see, e.g., Hames et al., 1990, Gel Electrophoresis of Proteins: A Practical Approach, IRL Press, New York; Shevchenko et al., 1996, Proc. Nat'l Acad. Sci. U.S.A. 93:1440-1445; Sagliocco et al., 1996, Yeast 12:1519-1533; Lander, 1996, Science 274:536-539; and Beaumont et al., Life Science News, 2001, 7, Amersham Pharmacia Biotech. The resulting series of protein “spots” on the second dimension SDS-PAGE gel can be measured to reveal the amount of one or more specific proteins in the mixture. The identity of the measured proteins may, or may not, be known; it is only necessary to be able to identify and measure specific protein “spots” on the second dimension gel. Numerous techniques are available to measure the amount of protein in a “spot” on the second dimension gel. For example, the gel can be stained with a reagent that binds to proteins and yields a visible protein “spot” (e.g., Coomassie blue dye, or staining with silver nitrate), and the density of the stained spot can be measured. Again by way of example, all, or most, proteins in a mixture can be measured with a fluorescent reagent before electrophoretic separation, and the amount of fluorescence in some, or all, of the resolved protein “spots” can be measured (see, e.g., Beaumont et al., Life Science News, 7, 2001, Amersham Pharmacia Biotech).

Again by way of example, any HPLC technique (e.g., exclusion chromatography, ion-exchange chromatography, hydrophobic interaction chromatography, reversed-phase chromatography and immobilized metal affinity chromatography) can be used to separate proteins in a mixture, and the separated proteins can thereafter be directed to a detector (e.g., spectrophotometer) that detects and measures the amount of individual proteins.

In some embodiments of the invention it is desirable to both identify and measure the amount of specific proteins. A technique that is useful in these embodiments of the invention is mass spectrometry, in particular the techniques of electrospray ionization mass spectrometry (ESI-MS) and matrix-assisted laser desorption/ionization mass spectrometry (MALDI-MS), although it is understood that mass spectrometry can be used only to measure the amounts of proteins without also identifying (by function and/or sequence) the proteins. These techniques overcame the problem of generating ions from large, non-volatile, analytes, such as proteins, without significant analyte fragmentation (see, e.g., Aebersold and Goodlett, 2001, Chem. Rev. 102: 269-296).

Thus, for example, proteins can be extracted from cells of a living thing and individual proteins purified therefrom using, for example, any of the art-recognized purification techniques described herein (e.g., HPLC). The purified proteins are subjected to enzymatic degradation using a protein-degrading agent (e.g., an enzyme, such as trypsin) that cleaves proteins at specific amino acid sequences. The resulting protein fragments are subjected to mass spectrometry. If the sequence of the complete genome (or at least the sequence of part of the genome) of the living thing from which the proteins were isolated is known, then computer algorithms are available that can compare the observed protein fragments to the protein fragments that are predicted to exist by cleaving the proteins encoded by the genome with the agent used to cleave the extracted proteins. Thus, the identity, and the amount, of the proteins from which the observed fragments are derived can be determined.

Again by way of example, the use of isotope-coded affinity tags in conjunction with mass spectrometry is a technique that is adapted to permit comparison of the identities and amounts of proteins expressed in different samples of the same type of living thing subjected to different treatments (e.g., the same type of living tissue cultured, in vitro, in the presence or absence of a candidate drug) (see, e.g., Gygi et al., 1999, Nat. Biotechnol. 17:994-999). In an exemplary embodiment of this method, two different samples of the same type of living thing are subjected to two different treatments (treatment 1 and treatment 2). Proteins are extracted from the treated living things and are labeled (via cysteine residues) with an ICAT reagent that includes (1) a thiol-specific reactive group, (2) a linker that can include eight deuteriums (yielding a heavy ICAT reagent) or no deuteriums (yielding a light ICAT reagent), and (3) a biotin molecule. Thus, for example, the proteins from treatment 1 may be labeled with the heavy ICAT reagent, and proteins from treatment 2 may be labeled with the light ICAT reagent. The labeled proteins from treatment 1 and treatment 2 are combined and enzymatically cleaved to generate peptide fragments. The tagged (cysteine-containing) fragments are isolated by avidin affinity chromatography (that binds the biotin moiety of the ICAT reagent). The isolated peptides are then separated by mass spectrometry. The quantity and identity of the peptides (and the proteins from which they are derived) may be determined. The method is also applicable to proteins that do not include cysteines by using ICAT reagents that label other amino acids.

### V. ESTIMATING HSD1 ACTIVITY

HSD1 encodes 11-β-Hydroxysteroid Dehydrogenase Type I, a component of glucose and lipid metabolism, catalyzing the conversion of inactive cortisone to active cortisol. Some embodiments of the present invention provide methods to estimate the activity of HSD1 based upon the level of expression of genes that are differentially expressed in a cell in response to cortisone. Marker genes identified as being differentially expressed in cells in response to cortisone may be used in a variety of nucleic acid detection assays to detect or quantify the expression level of a gene or multiple genes in a given sample. For example, traditional Northern blotting, nuclease protection, RT-PCR, differential display methods, and microarray may be used for detecting gene expression levels. Alternatively, protein detection methods such as 2D gel electrophoresis, surface-enhanced laser desorption/ionization time-of-flight (SELDI-TOF) followed by affinity chromatography, isotope-coded affinity tags (ICAT) in conjunction with mass spectrometry, may also be used for detecting gene expression levels. It is to be understood that alternative assay formats, other than the methodologies exemplified herein, may be used to estimate the activity of HSD1 based upon the expression of a plurality of gene markers identified in Table 2. Alternatively, HSD1 activity is estimated based upon the expression of a plurality of gene markers identified in Table 8.

As described in more detail in Example 2, genome-wide gene expression profiling was used to design a gene expression-based assay for measuring HSD1 activity to identify a general cortisone-induced gene expression signature in cultured HepG2 cells over-expressing HSD1. Cortisone is the substrate for HSD1 and the precursor to active cortisol. Some embodiments of the inventive methods are derived from a hypothesis that the cortisone response signature would be altered by active HSD1 inhibitors, but not by compounds that do not significantly alter the activity HSD1. Experimental validation of this hypothesis (see Example 2) confirms that it is possible to determine whether an agent inhibits HSD1 activity and to rank order HSD1 inhibitor agents based upon the degree to which the a plurality of genes selected from the cortisone response signature gene list in Table 2 is changed by each HSD1 inhibitory agent.

Based on the data described herein, a set of genes were identified, collectively referred to herein as a cortisone response signature (alternatively referred to as a cortisone response gene set), that are regulated in vitro in HepG2 cells over-expressing HSD1 in response to exposure to cortisone. Table 8 provides a list genes selected from Table 2 that may be used in the practice of some embodiments of the invention and represents an alternative cortisone response gene set.

In one embodiment of the invention, a method is provided for identifying a one or more genes useful for estimating HSD1 activity in a human subject. The method comprises: contacting a first sample of cells from a human hepatoma cell line with HSD1, wherein the cell line expresses HSD1; measuring an expression level for each of a plurality of genes of the cell line in the first sample; comparing the measured expression level of each of the plurality of genes in the first cell sample to a second expression level of each of the plurality of genes in a second sample of the cell line not contacted with cortisone; and identifying one or more genes in the plurality of genes that exhibit a statistically significant change in gene expression level between the first sample and the second sample, wherein the identified one or more genes are useful for estimating HSD1 activity in the human subject. In some embodiments the change in gene expression level is an at least 1.5-fold change between the first and second cell samples with a p-value for the change of less than or equal to 0.01. The plurality of genes whose expression is measured may be at least 10 genes, 50 genes, 100 genes, 500 genes, 1,000 genes, or 10,000 genes in the human genome. In one embodiment, the cell line is HepG2 modified to over-express HSD1. Optionally, both of the cell samples are contacted with forsoklin.

In another embodiment of the invention, a method of identifying an agent that modifies HSD1 activity is provided. The method comprises contacting an agent with a first cell sample of an HepG2 cell line, wherein the cell line over-expresses HSD1; and determining a change in HSD1 activity compared to a second sample of the cell line not contacted with the agent. HSD1 activity may be determined using an HSD1 enzyme assay or a gene expression measurement assay. As described previously, cell lines that over-express HSD1 may be produced in a variety of ways using a variety of different HSD1 gene sequences. In some embodiments the over-expressed HSD1 comprises a sequence encoding a polypeptide having at least 95% sequence identity to SEQ ID NO: 2. In some embodiments of the invention, HSD1 is produced in the HepG2 hepatoma cell line at least two-fold higher than expression of HSD1 in a HepG2 hepatoma cell line that does not over-express HSD1. Alternatively, HSD1 is expressed at least five-fold or at least ten-fold higher in the HepG2 hepatoma cell line than expression of HSD1 in a HepG2 hepatoma cell line that does not over-express HSD1. Optionally, the method may be practiced using samples of the HepG2 hepatoma cell line over-expressing HSD1 that are contacted with forskolin. Alternatively, or additionally, the samples of the HepG2 hepatoma cell line over-expressing HSD1 may be contacted with cortisone.

In another embodiment of the invention, changes in expression levels of cortisone response signature genes, used as a surrogate for HSD1 activity, may be measured in subjects other than HepG2 cells that over-express HSD1. One such exemplary method of estimating HSD1 activity in a subject comprising administering cortisone to a subject; obtaining an expression level for each of a plurality of genes in a test sample taken from the subject, wherein the subject has detectable HSD1 activity, and the plurality of genes correspond to at least three markers listed in Table 2; and then comparing each measured expression level for each of the plurality of genes in the test sample to a second set of gene expression levels for each of the plurality of genes measured in a control sample, wherein the control sample has not been contacted with cortisone. The difference in expression levels of the plurality of genes between the test sample and the control sample provides an estimate of HSD1 activity in the subject. In some embodiments the subject comprises a HepG2 hepatoma cell line, wherein the cell line over-expresses HSD1 at a level that is detectably higher than the level of HSD1 in HepG2 hepatoma cells that do not over-express HSD1.

The amount of cortisone to be added to a subject expressing HSD1 needs to be sufficient to allow the newly generated active glucocorticoid cortisol to activate the glucocorticoid receptor. The necessary amount of cortisol needed for glucocorticoid receptor activation will depend on factors such as gene sequence, the cell line, duration of incubation, HSD1 activity. Cortisone titration experiments may be performed to determine its saturation level or level at which HSD1 activity is maximized. Furthermore, other glucocorticoid precursors may be used in place of cortisone in the methods described herein, providing that the glucocorticoid precursors can be metabolized enzymatically by HSD1 to generate active glucocorticoid compounds. Examples of such glucocorticoid precursors include 11-dehydrocorticosterone, prednisone, or other pro-drugs, such as acetate.

In some embodiment of the invention, measuring HSD1 activity to identify a general cortisone-induced gene expression signature in cultured HepG2 cells over-expressing HSD1 is performed in the presence of forskolin. Forskolin, an activator of adenylyl cyclase, has been found to potentiate the glucocorticoid response in HepG2 cells (Ge et al., 2003, Endocrine Society Meeting, Poster P2-128).

As shown in the examples, active HSD1 inhibitor agents significantly alter the expression levels of the cortisone response signature genes whereas agents that do not inhibit HSD1 activity do not induce such changes in expression of genes in the cortisone response gene set. The identification and use of the cortisone response gene sets, as disclosed herein, provides an assay to monitor and classify cellular responses following exposure to agents that modulate HSD1 activity.

In some embodiments, polynucleotides that are complementary and hybridizable to the genes in the cortisone response signature listed in Table 2 and Table 8 are used to estimate HSD1 activity using the methods described in the examples. Persons of skill in the art will recognize that there are numerous examples of polynucleotides complementary and hybridizable to the cortisone response signature genes that may be used to measure gene expression levels, including oligonucleotide probes, RT-PCR primers.

Estimating HSD1 activity using cortisone response signature gene expression is useful, for example, in gene expression assays to diagnose and monitor treatment of diseases related to glucocorticoid metabolism and to identify and classify agents affecting HSD1 activity. Measurement of cortisone response signature gene expression can also be used to identify and categorize glucocorticoid disease subtypes not heretofore appreciated using conventional diagnostic tests. In some embodiments of the invention, expression of the cortisone response signature genes is measured using any of a variety of methods well known in the art, such as Northern blots, microarrays, and RT-PCR, and the like. For example, Table 8, sets forth primer sequences for a subset of the cortisone response genes listed in Table 2 that may be used in RT-PCR reactions to measure a gene expression level for each listed transcript. Cortisone response signature gene expression may also be monitored by other means, such as detecting their proteins or metabolic products.

One aspect of the present invention provides a method of estimating HSD1 activity in a subject. This is useful, for example, to detect changes in HSD1 activity in a subject in response to a disease state or treatment of a disease. The method comprises: administering cortisone to a subject; obtaining an expression level for each of a plurality of genes in a test sample taken from said subject, wherein said subject has detectable HSD1 activity, said plurality of genes corresponding to at least three markers listed in Table 2 or 8; and comparing each said measured expression level for said plurality of genes in said test sample to a second set of gene expression levels for each of said plurality of genes measured in a control sample, said control sample obtained from said subject not contacted with cortisone, wherein a difference in expression levels of said plurality of genes between said test sample and said control sample provides an estimate of HSD1 activity in said subject.

In another embodiment, a method is provided for estimating HSD1 activity in an HepG2 cell line that over-expresses HSD1. The method comprises: administering cortisone to a subject, said HepG2 cell line that over-expresses HSD1; obtaining an expression level for each of a plurality of genes in a test sample taken from said subject, wherein said subject has detectable HSD1 activity, said plurality of genes corresponding to at least three markers listed in Table 2 or 8; and comparing each said measured expression level for said plurality of genes in said test sample to a second set of gene expression levels for each of said plurality of genes measured in a control sample, said control sample obtained from said subject not contacted with cortisone, wherein a difference in expression levels of said plurality of genes between said test sample and said control sample provides an estimate of HSD1 activity in said subject.

Alternatively, the method of estimating HSD1 activity in a subject as described above may be used to identify a compound that modifies HSD1 activity, wherein said compound modifies the expression of one or more genes in said cortisone response signature in a sample from said cell line contacted with the compound in the presence of cortisone relative to the expression of one or more genes in said cortisone response signature in an analogous sample from said cell line not contacted with said compound in the presence of cortisone. A compound which modifies expression of one or more genes in said cortisone response signature compared to a control sample is said to modify HSD1 activity. In other embodiments of the invention, said cortisone response signature comprises at least 3 or more, at least 4 or more, and, at least 5 or more sequences selected from Table 2. In some embodiments of the invention, gene expression levels of the cortisone response signature are determined using a method selected from the group consisting of microarray, RT-PCR, Northern blot, 2D gel electrophoresis, SELDI-TOF, and ICAT.

The invention also provides for a method of classifying compounds that modify HSD1 activity comprising the steps of: a) contacting subject with a compound in the presence of cortisone; b) measuring gene expression level in a sample of mRNA, nucleic acid, or protein derived therefrom said subject, wherein said gene comprises a nucleotide sequence selected from cortisone response signature presented in Table 2 or Table 8; and c) comparing said measured expression level of said gene to the expression level of said gene in an analogous sample from said subject not contacted with said compound in the presence of cortisone, wherein the change in cortisone response signature gene expression level is used to rank order compounds that modify HSD1 activity. The above described method can also be performed wherein said subject comprises a HepG2 cell line that over-expresses HSD1.

### VI. IDENTIFYING OFF-TARGET ACTIVITY

Based on the data described herein, embodiments of the invention provide a method of identifying an off-target gene signature cells in the presence of an HSD1 compound, independent of cortisone exposure or HSD1 activity. In one embodiment, an off-target gene signature is detected in HepG2 cells over-expressing HSD1 in the presence of an HSD1 inhibitor compound and forskolin, independent of cortisone exposure. Changes in expression levels of the gene signature in response to the compound, independent of cortisone exposure, are indicators of off-target activity. Identification of the off-target signatures disclosed herein, the regulation of which is indicative of an HSD1 inhibitor compound's off-target activity, provides an assay to monitor and classify off-target responses following exposure to agents that modulate HSD1 activity.

Another embodiment provides methods for detection of off-target HSD1 compound activity. Off-target HSD1 activity is defined as changes in expression of genes that are induced independent of cortisone or HSD1 activity in the presence of an HSD1 compound. Thus, one embodiment provides a method of identifying one or more genes of a cell line as useful for estimating HSD1 off-target activity of an HSD1-agent. The method comprises contacting an HSD1-agent with a first sample of cells from a cell line, wherein said cell line does not express a detectable level of HSD1 activity, or, is not contacted with cortisone. Gene expression levels are then measured for a plurality of genes in the first cell sample and compared to the expression level of the same plurality of genes in a second sample cells of the cell line not contacted with the HSD1-agent. One or more genes are then identified in the plurality of measured genes which exhibit a statistically significant change in gene expression level between the first cell sample and the second cell sample, wherein the identified one or more genes are useful for estimating off-target activity of an HSD1-agent. In one embodiment the measured change in gene expression level is an at least 1.5-fold change with a p-value for the change of less than or equal to 0.01. In other embodiments, the plurality of genes for which gene expression levels are measured is at least 10 genes, 50 genes, 100 genes, 500 genes, 1000 genes, or at least 10,000 genes. In another embodiment, the cell line is HepG2. Alternatively, the cell line is a HepG2 cell line modified to over-express HSD1, in which case the cell samples are not contacted with cortisone. In other embodiments, the number of genes identified as showing a change in gene expression provides an estimate of the HSD1 off-target activity of the HSD1-agent. In this way embodiments of the inventive method can be used to compare two or more HSD1 inhibitor compounds to each other to determine which compound exhibits more or less HSD1 off-target activity.

In another embodiment of the invention, an off-target signature may be obtained in vitro in cell lines other than HepG2 that express HSD1 in the presence of a compound, independent of cortisone. In an alternative embodiment of the invention, an off-target signature may also be obtained in vitro in cell lines which do not express HSD1 in the presence of a compound. Changes in expression levels of the gene signature in response to the compound, independent of activation of the glucocorticoid receptor pathway, are indicators of off-target activity.

More specifically, an active HSD1 inhibitor agent, (3-(2-chlorophenyl)-4-methyl-5-(4-pentylbicyclo[2.2.2]oct-1-yl)-4H-1,2,4-triazole (described in U.S. Pat. No. 6,849,636 and WO2004058730A2)), that failed to achieve significant efficacy in vivo exhibited off-target activity in a HepG2 cell line that over-expresses HSD1. The presence of the off-target signature (see Table 6), as measured in a series of microarray based measurements of gene expression, was independent of cortisone treatment, suggesting that the off-target activity was occurring via a glucocorticoid independent pathway. This off-target gene signature was not detected in other active HSD1 inhibitor compounds with in vivo efficacy. Monitoring for the off-target signature identified by 3-(2-chlorophenyl)-4-methyl-5-(4-pentylbicyclo[2.2.2]oct-1-yl)-4H-1,2,4-triazole in the HepG2 cells over-expressing HSD1 can improve characterization and selection of candidate HSD1 compounds prior to in vivo efficacy testing.

The genes identified as being differentially expressed in cells in response to a compound and independent of cortisone may be used in a variety of nucleic acid detection assays to detect or quantify the expression level of a gene or multiple genes in a given sample. For example, traditional Northern blotting, nuclease protection, RT-PCR, differential display methods, and microarray may be used for detecting gene expression levels. Alternatively, protein detection methods such as 2D gel electrophoresis, surface-enhanced laser desorption/ionization time-of-flight (SELDI-TOF) followed by affinity chromatography, isotope-coded affinity tags (ICAT) in conjunction with mass spectrometry, may also be used for detecting gene expression levels. It is to be understood that alternative assay formats, other than the methodologies exemplified herein, may be used to estimate a compound's off-target activity based upon the expression of a plurality of genes listed in Table 6 or 9.

One aspect of the invention provides for a method of classifying compounds that modify HSD1 activity by monitoring off-target activity comprising: a) contacting a compound with a HepG2 cell line over-expressing HSD1; b) measuring gene expression level in a sample of mRNA, nucleic acid, or protein derived therefrom said cell line, wherein said gene comprises a nucleotide sequence selected from the off-target signature genes presented in Table 6 or Table 9; and c) comparing said measured expression level of said gene to the expression level of said gene in an analogous cell line not contacted with said compound; wherein a change in off-target gene expression is indicative of off-target activity.

### VII. REAL-TIME PCR TO SCREEN LARGE PANEL OF COMPOUNDS FOR MODULATION OF HSD1 ACTIVITY OR OFF-TARGET ACTIVITY

In practice, a gene expression-based pharmacodynamic assay based on a small number of genes can be performed with relatively little effort using existing quantitative real-time PCR technology familiar to clinical laboratories. Quantitative real-time PCR measures PCR product accumulation through a dual-labeled fluorigenic probe. A variety of normalization methods may be used, such as an internal competitor for each target sequence, a normalization gene contained within the sample, or a housekeeping gene. Sufficient RNA for real time PCR can be isolated from low milligram quantities from a subject. Quantitative thermal cyclers may now be used with microfluidics cards preloaded with reagents making routine clinical use of multigene expression-based assays a realistic goal.

The cortisone signature or off-target signature gene sets or a subset of genes selected from the cortisone or off-target gene sets, which are assayed according to the present invention are typically in the form of total RNA or mRNA or reverse transcribed total RNA or mRNA. General methods for total and mRNA extraction are well known in the art and are disclosed in standard textbooks of molecular biology, including Ausubel et al., Current Protocols of Molecular Biology, John Wiley and Sons (1997). RNA isolation can also be performed using purification kit, buffer set and protease from commercial manufacturers, such as Qiagen (Valencia, Calif.) and Ambion (Austin, Tex.), according to the manufacturer's instructions.

TAQman quantitative real-time PCR can be performed using commercially available PCR reagents (Applied Biosystems, Foster City, Calif.) and equipment, such as ABI Prism 7900HT Sequence Detection System (Applied Biosystems) according the manufacturer's instructions. The system consists of a thermocycler, laser, charge-coupled device (CCD), camera, and computer. The system amplifies samples in a 96-well or 384-well format on a thermocycler. During amplification, laser-induced fluorescent signal is collected in real-time through fiber-optics cables for all 96 wells, and detected at the CCD. The system includes software for running the instrument and for analyzing the data.

Based upon the cortisone response and off-target gene signatures identified in the present invention, a real-time PCR TAQman assay can be used to screen a large panel of compounds for inhibition of HSD1 activity (repression of the cortisone response signature) as well as for off-target activity. Oligonucleotide primer and probes that are complementary to or hybridize to the cortisone response were designed and are set forth in Tables 2 and 7. Oligonucleotide primer and probes that are complementary to or hybridize to the off-target signature genes were designed and are set forth in Tables 5 and 8. Of course, as would be apparent to a person of skill in the art, many other oligonucleotide primers and probe sequences could be selected and used in the practice of these methods based upon the transcript sequences set forth in the Sequence Listing as identified in Table 2 for the cortisone signature genes and in Table 6 for the off-target signature genes.

In an embodiment of the invention, the cortisone signature response genes selected for measurement of gene expression in a real-time PCR assay comprises: Homer-2, PGC, G6PC, TAT, and LIPG. Homer-2 refers to Homer homolog 2 protein. Homer-2 reference transcripts are represented by NM_{—}004839 which encodes NP_{—}004830 (transcript variant 1), NM_{—}199330 which encodes NP_{—}955362 (transcript variant 2), NM_{—}199331 which encodes NP_{—}955363 (transcript variant 3), and NM_{—}199332 which encodes NP_{—}955364 (transcript variant 4). Homer-2 nucleic acid sequences are provided by SEQ ID NO:s: 12, 13, 14, and 15. Homer-2 amino acid sequences are provided by SEQ ID NO:s: 16, 17, 18, and 19. PGC refers to progastricsin, also known as pepsinogen C. PGC reference transcript is represented by NM_{—}002630 which encodes NP_{—}002621. PGC nucleic acid sequence is provided by SEQ ID NO: 20. PGC amino acid sequence is provided by SEQ ID NO: 21. G6PC refers to glucose-6-phosphatase, catalytic. G6PC reference transcript is represented by NM_{—}000151 which encodes NP_{—}000142. G6PC nucleic acid sequence is provided by SEQ ID NO: 22. G6PC amino acid sequence is provided by SEQ ID NO: 23. TAT refers to tyrosine aminotransferase. TAT reference transcript is represented by NM_{—}000353 which encodes NP_{—}000344. TAT nucleic acid sequence is provided by SEQ ID NO: 24. TAT amino acid sequence is provided by SEQ ID NO: 25. LIPG refers to lipase, endothelial. LIPG reference transcript is represented by NM_{—}006033 which encodes NP_{—}006024. LIPG nucleic acid sequence is provided by SEQ ID NO: 26. LIPG amino acid sequence is provided by SEQ ID NO: 27.

In another embodiment of the invention, the off-target signature genes selected for measurement of gene expression in a real-time PCR assay comprises: DHCR7, SCD, ACLY, FBXO9, and HEY1. DHCR7 refers to 7-dehydrocholesterol reductase. DHCR7 reference transcript is represented by NM_{—}001360 which encodes NP_{—}001351. DHCR7 nucleic acid sequence is provided by SEQ ID NO: 28. DHCR7 amino acid sequence is provided by SEQ ID NO: 29. SCD refers to stearoyl-CoA desaturase, also known as delta-9-desaturase. SCD reference transcript is represented by NM_{—}005063 which encodes NP_{—}005054. SCD nucleic acid sequence is provided by SEQ ID NO: 30. SCD amino acid sequence is provided by SEQ ID NO: 31. ACLY refers to ATP citrate lyase. ACLY reference transcripts are represented by NM_{—}001096 which encodes NP_{—}001087 (transcript variant 1) and NM_{—}198830 which encodes NP_{—}942127 (transcript variant 2). ACLY nucleic acid sequences are provided by SEQ ID NOs: 32 and 33. ACLY amino acid sequences are provided by SEQ ID NOs: 34 and 35. FBXO9 refers to F-box protein 9. FBXO9 reference transcripts are represented by NM_{—}012347 which encodes NP_{—}036479 (transcript variant 1), NM_{—}033480 which encodes NP_{—}258441 (transcript variant 2), and NM_{—}033481 which encodes NP_{—}258442 (transcript variant 3). FBXO9 nucleic acid sequences are provided by SEQ ID NOs: 36, 37, and 38. FBXO9 amino acid sequences are provided by SEQ ID NOs: 39, 40, and 41. HEY1 refers to hairy/enhancer-of-split related with YRPW motif 1. HEY1 reference transcript is represented by NM_{—}012258 which encodes NP_{—}036390. HEY1 nucleic acid sequence is provided by SEQ ID NO: 42. HEY1 amino acid sequence is provided by SEQ ID NO: 43. It is to be understood that other assay formats, other than real-time PCR, may be used to estimate HSD1 activity and off-target activity based upon the expression of a plurality of genes identified in Table 2 or Table 6, respectively.

### EXAMPLES

Examples are provided below to further illustrate different features and advantages of the present invention. The examples also illustrate useful methodology for practicing the invention. These examples do not limit the claimed invention.

### Example 1

### Generation of HepG2 Cells Over-Expressing HSD1

HepG2 cells express low levels of endogenous HSD1 (Williams et al., 2000, J. Biol. Chem. 275:30232-30239; Parks et al., 1998, J Steroid Biochem Mol. Biol. 1998 67:341-346). HepG2 hepatoma cells (ATCC HB-8065; Knowles et al., 1980, Science 209:497-499) were transfected with cDNA encoding full-length human HSD1 (SEQ ID NO: 2), as described in more detail below, to obtain a modified HepG2 cell line that over-expresses HSD1.

A. PCR. The HSD1 cDNA sequence was cloned from human liver Quick-Clone cDNA (Clontech, Palo Alto, Calif.) using the polymerase chain reaction (PCR). For PCR, 0.5 μl (0.5 ng) cDNA was added to 41 μl of water, 5 μl of 10×NE Buffer for Vent polymerase (New England Biolabs, Beverly, Mass.), 1 μl of 10 mM dNTPs and 0.5 μl of Vent Polymerase (New England Biolabs, Beverly, Mass.). PCR was done in a Perkin-Elmer Cetus DNA Thermal Cycler using 1 μM each of the HSD1 “forward” and “reverse” primers for HSD1 (HSD1 forward primer 5′-CCT GTC GGA TGG CTT TTA TG-3′ (SEQ ID NO: 44); HSD1 reverse primer 5′-GCG GTG CAT GAC ATT CAT TA-3′ (SEQ ID NO: 45) in a total volume of 50 μl. Thirty cycles of amplification were performed using a 1 min denaturation at 94° C. followed by 1 min annealing at 55° C. and a 1 min synthesis at 72° C. The 30 cycles of PCR were followed by a 10 minute extension at 72° C. The 50 μl reaction was then chilled to 4° C. 10 μl of the resulting reaction product was run on a 1% Seakem Gold agarose (Cambrex) gel stained with 0.3 μg/ml ethidium bromide (Fisher Biotech, Fair Lawn, N.J.). Nucleic acid bands in the gel were visualized and photographed on a UV light box to determine if the PCR had yielded products of the expected size, in the case of the HSD1 mRNA, a product of about 1083 base pairs. The remainder of the 50 μl PCR reactions was purified using the Wizard PCR purification kit (Promega, Madison, Wis.) following the QIAquik PCR Purification Protocol provided with the kit. The purified PCR product was ligated into the pPCR-Script Amp vector and transformed into XL-1 Blue MRF' Kan using the PCR-Script Amp cloning kit from Stratagene (LaJolla, Calif.). Ampicillin-resistant clones were selected on LB agar plates containing 100 μg/ml ampicillin, 50 μg/ml IPTG and 40 μg/ml X-Gal.

B. Cloning and HepG2 transfection. About 4 μl of purified HSD1 PCR product from human liver cDNA were used in a ligation reaction using the reagents and instructions provided with the PCR-Script Amp cloning kit (Stratagene, LaJolla, Calif.). About 2 μl of the ligation reaction was used following the manufacturer's instructions to transform XL-1 Blue MRF' Kan electroporation-competent E. coli provided with the cloning kit. After the 1 hour recovery of the cells in SOC medium (provided with the PCR-Script Amp cloning kit), 200 μl of the mixture was plated on LB medium agar plates (Sambrook, et al., in Molecular Cloning, A Laboratory Manual, 2^{nd} Edition, Cold Spring Harbor Laboratory Press, 1989) containing 100 μg/ml Ampicillin, 50 μg/ml Isopropyl-beta-D-thiogalactopyranoside and 40 μg/ml X-Gal (5-bromo-4-chloro-3-indolyl-beta-D-galactoside (Sigma, St. Louis, Mo.). Plates were incubated overnight at 37° C. White colonies were picked from the plates into 3 ml of LB medium containing 100 μg/ml Ampicillin. These liquid cultures were incubated shaking at 300 rpm overnight at 37° C. Plasmid DNA was extracted from these cultures using the Qiagen (Valencia, Calif.) Qiaquick Spin Miniprep kit (Qiagen). Five putative HSD1 clones were identified and prepared for a restriction enzyme digest reaction to confirm the presence of the expected HSD1 sequence and orientation within the vector. Five μl of each each miniprep DNA was mixed with 5 U HindIII, 5 U SphI, 2.5 μl 10×NEBuffer 2 (all reagents from New England Biolabs) in a final volume of 25 μl for 1 hrs at 37° C. The reaction was run on a 1% agarose gel and the DNA bands generated by the PCR reaction were visualized and photographed on a UV light box to determine which minipreps samples had PCR product of the size predicted for the corresponding HSD1 cDNA. An appropriate clone was identified and 25 μl of the miniprep DNA was mixed with 25 U HindIII, 25 U NotI, 5 μl 10×NEBuffer 2, 0.1 mg/ml bovine serum albumin (all reagents from New England Biolabs) in a final volume of 50 μl for 3 hrs at 37° C. The restriction enzyme fragment containing HSD1 cDNA was purified by agarose gel purification with the Qiaquick Gel Extraction Kit (Qiagen) into 50 μl of buffer EB contained in the kit. Approximately 6 μg of plasmid pcDNA3.1(+) (Invitrogen, Carlsbad, Calif.) was mixed with 25 U HindIII, 25 U NotI, 5 μl 10×NEBuffer 2, 0.1 mg/ml bovine serum albumin (all reagents from New England Biolabs) in a final volume of 50 μl for 3 hrs at 37° C. and purified by agarose gel purification with the Qiaquick Gel Extraction Kit (Qiagen) into 50 μl of buffer EB. Five p. 1 of the gel-purified, restriction enzyme-treated HSD1 (equivalent to approximately 20 ng) was used in a ligation reaction containing 5 μl of gel-purified, restriction enzyme-treated pcDNA3.1(+) (approximately 25 ng) using 1 μl of T4 DNA ligase and, 1.2 μl of 10× ligation buffer containing 5 mM ATP (all reagents from Promega, Madison, Wis.) in a final volume of 12.2 μl. The ligation reaction was carried out by incubating the samples overnight at 16° C. About 1 μl of the ligation reaction was used to transform NovaBlue chemically competent E. coli from Novagen (EMD Biosciences, Madison, Wis.). After the 1 hour recovery of the cells in SOC medium, the mixture was plated on LB medium agar plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.). Plates were incubated overnight at 37° C. White colonies were picked from the plates into 3 ml of LB medium containing 100 μg/ml Ampicillin. These liquid cultures were incubated shaking at 300 rpm overnight at 37° C. Plasmid DNA was extracted from these cultures using the Qiaquick Spin Miniprep kit (Qiagen, Valencia, Calif.). Six putative HSD1 clones were identified and prepared for a restriction enzyme digest reaction to confirm the presence of the expected HSD1 sequence and orientation within the vector. Five μl of each miniprep DNA was mixed with 5 U HindIII, 5 U NotI, 2.5 μl 10×NEBuffer 2, 0.1 mg/ml bovine serum albumin (all reagents from New England Biolabs) in a final volume of 20 μl for 1 hr at 37° C. The reaction was run on a 1% agarose gel and the DNA bands generated by the PCR reaction were visualized and photographed on a UV light box to determine which minipreps samples had PCR product of the size predicted for the corresponding HSD1 cDNA. DNA sequence analysis of the HSD1 cloned DNAs confirmed a polynucleotide sequence representing the full length coding region of HSD1.

Once an appropriate pcDNA3.1/HSD1 clone had been identified, purified plasmid DNA was prepared for mammalian cell transfection into HepG2 hepatoma cells (ATCC HB-8065) according to manufacturer's instructions using the FuGene 6 transfection reagent (Roche Applied Sciences, Indianapolis, Ind.). Stable transfectants of HepG2 cells were selected with Geneticin (0.8 mg/ml; Invitrogen, Carlsbad, Calif. in culture medium (Dulbecco's Modified Eagle Medium with high glucose containing 2 mM L-glutamine, 10% fetal bovine serum, 100 U/ml penicillin, 100 μg/ml Streptomycin, 10 μM sodium pyruvate, 100 μM non-essential amino acids, 0.003 N NaOH). HepG2 transfectants that over-expressed HSD1 are hereafter referred to as “HepG2/HSD1 cells.”

C. Confirmation of HSD1 expression in HepG2 cells. Expression of HSD1 in transfectants was confirmed by assessment of the oxidoreductase activity in cells using [³H]-cortisone as the reaction substrate (Schweizer et al., 2003, Mol. Cell. Endocrinol. 212:41-49; Alberts et al., 2002, Diabetologia 45:1528-1532; Kotelevtsev et al., 1997, Proc. Natl. Acad. Sci. 94:14924-14929), and by mRNA analysis.

Oxidoreductase Method: [1,2-³H]-cortisone (48.7 Ci/mmol) was added to a final concentration of 20 nM to HepG2/HSD1 (2×10⁶ cells/well in 6-well plates) and incubated for 1.5, 3, 6 and 22 hrs at 37° C. in culture medium (Dulbecco's Modified Eagle Medium with high glucose containing 2 mM L-glutamine, 10% fetal bovine serum, 100 U/ml penicillin, 100 μg/ml Streptomycin, 10 μM sodium pyruvate, 100 μM non-essential amino acids, 0.003 N NaOH and 0.8 mg/ml Geneticin). The samples (100 μl aliquots) were then extracted with 300 μl ethyl acetate and the upper, organic phases were dried. The dried organic extracts were then resuspended either in 100 μl DMSO for analysis by reverse-phase HPLC (buffer A=10% methanol/90% water; buffer B=90% methanol/10% water using a gradient from 20-45% Buffer B; YMC Combiscreen Pro C18 50 mm×4.6 mm column; flow rate 2.5 mL/min).

mRNA Analysis: mRNA prepared from transfectants was used to detect HSD1 expression. More specifically, total RNA was isolated from HepG2/HSD1 cells using the RNeasy 96-Kit (Qiagen Inc., Valencia, Calif.) following the manufacturer's instructions, including the optional on-column removal of contaminating DNA step by RNase-free DNase I treatment. The RNA was reverse-transcribed to single stranded cDNA using random hexamer primers with TAQman reverse transcription reagents (Applied Biosystems, Foster City, Calif.).

The sequences of the TAQman primers and probes used to detect the target cDNAs are shown in Table 1. Oligonucleotide probes were labeled with the 6-FAM fluorophore at the 5′ end (FAM) and a non-fluorescent quencher at the 3′ end (NFQ). Real-time PCR was performed on human HepG2/HSD1 cDNA using the TaqMan PCR Reagent Kits (Applied Biosystems, Foster City, Calif.) and appropriate primer/probe sets on an ABI Prism 7900HT Sequence Detection System (Applied Biosystems, Foster City, Calif.) in the 384-well format according to manufacturer's instructions. The levels of mRNA were normalized to the amount of 18S RNA detected in each sample using the Eukaryotic 18S rRNA Endogenous Control from Applied Biosystems.

TABLE 1
TAQman primers and probes used to detect HSD1.

| Primer | Sequence | SEQ ID NO: |
|---|---|---|
| Forward | GGAAGCATTGTTGTCGTCTCC | SEQ ID NO: 46 |
| Reverse | CTTGCTTGCAGAATAGGCAGC | SEQ ID NO: 47 |
| Probe | FAM-CTCTGGCTGGGAAAGTGGCTTATC | SEQ ID NO: 48 |
| | CAAT-TAMRA | |

Alternatively, HSD1 specific antibodies may be used to detect HSD1 expression in a subject using specific protein binding assays such as ELISA, Western blot, immunofluorescent microscopy, or immunohistochemical methods. HSD1 antibodies may be obtained from Cayman Chemical (Ann Arbor, Mich.) or The Binding Site (Birmingham, England). Methods of use of HSD1-specific antibodies to determine HSD1 localization and distribution have been described (Ricketts et al., 1998, J. Clin. Endocrinol. Metab. 83:1325-1335; Suzuki et al., 2001, Mol. Cell. Endocrinol. 173:121-125).

### Example 2

### Determination of Cortisone Response Signature Genes

HepG2 human hepatoma cells stably transfected with human 11βHSD1 (HepG2/HSD1 cells) were plated at 2×10⁶ cells per well on 6-well plates in 1 ml Culture Medium (DMEM with 2 mM L-Glutamine, 10% charcoal-dextran-treated FBS, 100 U/ml Penicillin, 100 μg/ml streptomycin, 10 μM sodium pyruvate, 100 μM non-essential amino acids, 0.003 N NaOH) at 37° C./5% CO₂. After 24 hours, the Culture Medium was replaced with 500 μl Culture Medium containing 1000 nM cortisone (500 nM final concentration) and 20 μM forskolin (10 μM final concentration). The cells were incubated for 20 hrs at 37° C./5% CO₂. Samples were run in triplicates.

Total RNA isolated from cultured cell samples was used to make fluorescently labeled cRNA that was hybridized to DNA oligonucleotide microarrays as described previously (Hughes et al., supra., Marton et al., 1998, Nat. Med. 4:1293-301). Briefly, 4 μg of total RNA from each cell culture sample was used to synthesize dsDNA through RT. cRNA was produced by in vitro transcription and labeled postsynthetically with Cy3 or Cy5. Two populations oflabeled cRNA, a control population from vehicle/forskolin treated cells and an experimental population, i.e., cortisone/forskolin plus vehicle or plus HSD1 test compound, were compared with each other by competitive hybridization to microarrays. Two hybridizations were done with each cRNA sample pair using a fluorescent dye reversal strategy.

The human microarrays used in this study contained 23,653 oligonucleotide probes corresponding to individual genes or expressed sequence tags. Oligonucleotide probe sequences were chosen to maximize gene specificity and minimize the 3′ replication bias inherent in RT of mRNA. In addition, the microarray format contained 2,107 control probes for quality control purposes. All oligonucleotide probes on the microarrays were synthesized in situ with inkjet technology (Agilent Technologies, Palo Alto, Calif.; Hughes et al., supra.).

After hybridization, arrays were scanned and fluorescence intensities for each probe were recorded. Ratios of transcript abundance (experimental to control) were obtained following normalization and correction of the array intensity data. Gene expression data analysis was done with the ROSETTA RESOLVER® gene expression analysis software (version 3.2, Rosetta Biosoftware, Seattle, Wash.). For each gene sequence present on the microarrays, statistical significance of differential gene expression was determined by calculating P values according to the following equation:

P value=2×(1−Erf(|xdev|))

where Erf is the error function for a Gaussian distribution of zero mean and xdev is the adjusted difference in fluorescence intensities between Cy3 and Cy5 signals calculated by the equation:

$$xdev = \frac{r - g}{\sqrt{{σ}_{r}^{2} + {σ}_{g}^{2}}}$$

where r is Cy5 intensity, g is Cy3 intensity, and u is the error associated with the respective channel.

Gene expression signatures elicited by cortisone alone were identified by microarray analysis of HepG2/HSD1 cells incubated with cortisone and forskolin for 20 hrs and compared to HepG2/HSD1 cells incubated with forskolin alone. The on-target signature is triggered by the cortisone treatment. There is no cortisone response signature without cortisone treatment. To broadly identify the genes responding to the cortisone treatment three replicates of gene expression measurements of cortisone treated HepB2/HSD1 cells were combined in an error weighted fashion using an error model developed for two-color hybridization experiments (see Parrish et al., 2004 J. Neurosci. Methods, 132:57-68) using Rosetta error model and selected those with p-value <0.01. This selection of genes is called the cortisone signature genes. The 77 cortisone response signature genes which exhibited a detectable change in expression level in response to cortisone with a p-value of less than 0.01 are presented in Table 2.

TABLE 2
Cortisone response signature gene markers.

| Reference | | | | |
|---|---|---|---|---|
| Transcript | Gene Name | SEQ ID NO: | Probe Sequence | SEQ ID NO: |
| NM_002165 | ID1 | SEQ ID NO: 49 | CAGAGACTTTAGGGGGTGGGATTCCACTCGTGTGT | SEQ ID NO: 50 |
| | | | TTCTATTTTTTGAAAAGCAGACATT | |
| NM_007256 | SLCO2B1 | SEQ ID NO: 51 | TGCTGGTTGGTATCTGTAAATGTTTAATAAATATC | SEQ ID NO: 52 |
| | | | TGAGCATGTATCTATCAACGCCAAG | |
| NM_005195 | CEBPD | SEQ ID NO: 53 | GGTAGCTTTTTCTACATCTTACTCCTGTTGATGCA | SEQ ID NO: 54 |
| | | | GCTAAGGTACATTTGTAAAAAGAAA | |
| NM_021175 | HAMP | SEQ ID NO: 55 | TATTTATTCCTGCTGCCCCAGAACATAGGTCTTGG | SEQ ID NO: 56 |
| | | | AATAAAATGGCTGGTTCTTTTGTTT | |
| AK095358 | | SEQ ID NO: 57 | TTTCCTCTAACCAAAATATACGTGTAGCCATTACC | SEQ ID NO: 58 |
| | | | AATGTACAACTCTCAATGCGGAGTT | |
| AK000757 | SORT1 | SEQ ID NO: 59 | CTATTCCCTTAGGACAGAAAATTGCATGTGAGGTG | SEQ ID NO: 60 |
| | | | GGATAATCGAGTTTCAGTGACCCAC | |
| NM_000429 | MAT1A | SEQ ID NO: 61 | CACACTCGACATGAAAATTCAGAATTTTATACTTT | SEQ ID NO: 62 |
| | | | CCCTACCCTCTAGAGAAATAAGATC | |
| NM_001353 | AKR1C1 | SEQ ID NO: 63 | CCCCTAATTATCCATTTTCTGATGAATATTAACAT | SEQ ID NO: 64 |
| | | | GGAGGGCATTGCATGAGGTCTGCCA | |
| NM_012242 | DKK1 | SEQ ID NO: 65 | GATGCTATGAAAACCTTTTATGACCTTCATCAACT | SEQ ID NO: 66 |
| | | | CAATCCTAAGGATATACAAGTTCTG | |
| BG539635 | IMAGE: 4692283 | SEQ ID NO: 67 | CCTGAGCTTAGTCTAGTAGGAGGTGACAGATTTGT | SEQ ID NO: 68 |
| | | | AAATAATAGATTTGAAGTTGCTGAG | |
| NM_002616 | PER1 | SEQ ID NO: 69 | GATGGCATTTGTGTTTTTGATATTTGTGTCTGTTA | SEQ ID NO: 70 |
| | | | CTACTTTTTTAATACAAAAAGATAA | |
| NM_000670 | ADH4 | SEQ ID NO: 71 | CCCTGCCTTTTGACAAAATCAGTGAGGCATTTGAC | SEQ ID NO: 72 |
| | | | CTAATGAACCAAGGAAAAAGCGTCC | |
| NM_001876 | CPT1A | SEQ ID NO: 73 | GCTGACCGGTGCATGTAAATACAGTATGCTCTTTG | SEQ ID NO: 74 |
| | | | GATGTAAATCTTAGAAATGCAGTGT | |
| NM_001860 | SLC31A2 | SEQ ID NO: 75 | GGAAAGAGAGAAAATCACTGCTGTATACTAAATAC | SEQ ID NO: 76 |
| | | | CTCACAGATTAGATGAAAAGATGGT | |
| XM_032571 | KIAA0888 | SEQ ID NO: 77 | AGACATTGAATGACTTTGTTAAAGGCACAATTAAT | SEQ ID NO: 78 |
| | | | CACATTGGTTGTACTCTGAAGACAG | |
| NM_002083 | GPX2 | SEQ ID NO: 79 | CCTTAAAGTTGCCATATAGATGTGAACTGCTCAAC | SEQ ID NO: 80 |
| | | | ACACAGATCTCCTACTCCATCCAGT | |
| NM_000151 | G6PC | SEQ ID NO: 22 | TGTTTTTCAATCTCATCTGATATGCAGAGTATTTC | SEQ ID NO: 81 |
| | | | TGCCCCACCCACCTACCCCCCAAAA | |
| NM_000689 | ALDH1A1 | SEQ ID NO: 82 | CATCCAGGATTTTTGTGGAAGAATCAATTTATGAT | SEQ ID NO: 83 |
| | | | GAGTTTGTTCGAAGGAGTGTTGAGC | |
| NM_017983 | WIPI49 | SEQ ID NO: 84 | GCGCGGGAGACATTGTATACACATCATGCTATTTA | SEQ ID NO: 85 |
| | | | AAATACGTTCAAACTATAGTGTAAA | |
| AL117595 | COPEB | SEQ ID NO: 86 | TCGGACACACTAAGGTTTTGATTTTGAATTTCAGC | SEQ ID NO: 87 |
| | | | CTTATTAGAAGATCTAACCTAAGAG | |
| AB037805 | KLHL14 | SEQ ID NO: 88 | TCAGAACCTTTTGTTGTCAACTGTCACTGTTTCTT | SEQ ID NO: 89 |
| | | | CCAAAAGTCACAATTTGTACAAGAA | |
| NM_000240 | MAOA | SEQ ID NO: 90 | TGATAGAATAAAGCCTTGTGATCACTTTCTGAAAT | SEQ ID NO: 91 |
| | | | TCACAAAGTTAAACGTGATGTGCTC | |
| NM_017526 | OBRGRP | SEQ ID NO: 92 | CACTTTAAGAAAGACTTCATAAGTAGGAGATGAGT | SEQ ID NO: 93 |
| | | | TTTATTCTCAGCAAATAGACCTGTC | |
| NM_001354 | AKR1C2 | SEQ ID NO: 94 | TGATCCCAAAAGATGAAAATGGAAAAATACTATTT | SEQ ID NO: 95 |
| | | | GACACAGTGGATCTCTGTGCCACAT | |
| NM_005763 | AASS | SEQ ID NO: 96 | AGGCAATACACCTCTGAACATGTGTGTGATAAATG | SEQ ID NO: 97 |
| | | | GGTTTGCTAATGTGCTGTTTTAAAG | |
| NM_017791 | C14orf58 | SEQ ID NO: 98 | CAAGGAACATGCTGTATGTAATAGAAGAAAGAAGT | SEQ ID NO: 99 |
| | | | CCACGTTTTCGGCAGAAGTAGTGAG | |
| AB033025 | KIAA1199 | SEQ ID NO: 100 | CATATGTTTCACAGTACAGGATCTGTACATAAAAG | SEQ ID NO: 101 |
| | | | TTTCTTTCCTAAACCATTCACCAAG | |
| NM_004117 | FKBP5 | SEQ ID NO: 102 | CCCAACTCAGGACAGAACAGTGTTTAATGTAAAGT | SEQ ID NO: 103 |
| | | | TTGTTATAGTCTATGTGATTCTGGA | |
| NM_002630 | PGC | SEQ ID NO: 20 | AGGATGAGTATGGACAGTTTCTCGTGAACTGTAAC | SEQ ID NO: 104 |
| | | | AGCATTCAGAATCTGCCCAGCTTGA | |
| NM_002069 | GNAI1 | SEQ ID NO: 105 | GGTGATGGCTTTGGAAATAACATAAATATACCTTG | SEQ ID NO: 106 |
| | | | TACTGAATGACAGACTATTACTACG | |
| NM_002153 | HSD17B2 | SEQ ID NO: 107 | GCTCACTATTTGCCTATTGGCATATATGATTACTT | SEQ ID NO: 108 |
| | | | TGCTAAAAGACATTTTGGCCAAGAC | |
| NM_001424 | EMP2 | SEQ ID NO: 109 | ATATGTACCAACAACACGAATTGCACAGTCATCAA | SEQ ID NO: 110 |
| | | | TGACAGCTTTCAAGAGTACTCCACG | |
| NM_013261 | PPARGC1A | SEQ ID NO: 111 | CTGTAGTCTAAGACCTGATCTATAGATACCTAGAA | SEQ ID NO: 112 |
| | | | TAGCCATGTACTATAATGTGATGAT | |
| NM_014899 | RHOBTB3 | SEQ ID NO: 113 | TATATCAGAAACTCACAAACCTAGACATGGAAAAA | SEQ ID NO: 114 |
| | | | CAGATTACTGTCTATTGTCAGCATC | |
| NM_000577 | IL1RN | SEQ ID NO: 115 | CAACCAACTAGTTGCTGGATACTTGCAAGGACCAA | SEQ ID NO: 116 |
| | | | ATGTCAATTTAGAAGAAAAGATAGA | |
| NM_017786 | FLJ20366 | SEQ ID NO: 117 | TTGCCGTTTTAAAATGTGTAATTGTTCCAGCATTC | SEQ ID NO: 118 |
| | | | CAATGGTCTTGTGCATAGCAGGGGA | |
| NM_002108 | HAL | SEQ ID NO: 119 | TCTTCAGCTATTAATTGTAGAGGTGATTAAAACCA | SEQ ID NO: 120 |
| | | | ACAAGGAGTTTCATGTGCAAAGGAG | |
| AA005351 | | SEQ ID NO: 121 | ATAATACCAAAGCAACCAAACCCATTAGCACCATT | SEQ ID NO: 122 |
| | | | CCATCCTATATGAGACCTTGCTTGG | |
| NM_006033 | LIPG | SEQ ID NO: 26 | AAGGACATTGTTTTAATCTGTATCGTGCCAAAGTT | SEQ ID NO: 123 |
| | | | GTATCACTGTTAAACTTCTGAAGAC | |
| NM_000242 | MBL2 | SEQ ID NO: 124 | AAGTATCAGGATACTATATTTTAGGTCTTAATACA | SEQ ID NO: 125 |
| | | | CACAGTTATGCCGCTAGATACATGC | |
| NM_016569 | TBX3 | SEQ ID NO: 126 | TTGGGTTTATTTCCAGGTCACAGAATTGCTGTTAA | SEQ ID NO: 127 |
| | | | CACTAGAAAACACACTTCCTGCACC | |
| NM_004657 | SDPR | SEQ ID NO: 128 | TTAATTTGCTCTTAGGTTCTATCTCTGTAGAATGT | SEQ ID NO: 129 |
| | | | CTCCAAGATTGAAGAAGAAACTGAG | |
| NM_005524 | HES1 | SEQ ID NO: 130 | AGTCATCAAAGCCTATTATGGAGAAAAGACGAAGA | SEQ ID NO: 131 |
| | | | GCAAGAATAAATGAAAGTCTGAGCC | |
| NM_004812 | AKR1B10 | SEQ ID NO: 132 | TTGGAAGACTATCCCTTCGATGCAGAATATTGAGG | SEQ ID NO: 133 |
| | | | TTGAATCTCCTGGTGAGATTATACA | |
| NM_004694 | SLC16A6 | SEQ ID NO: 134 | TTCTTTCTTCCTATGGTCTTGTCTGAATAAACTAC | SEQ ID NO: 135 |
| | | | TCTCCTGAATAAAACAACATCCAAC | |
| NM_001875 | CPS1 | SEQ ID NO: 136 | TGGCTAATCCAGGAATATTGTTATCCCTTCCCATT | SEQ ID NO: 137 |
| | | | ATATTGAAGTTGAGAAATGTGACAG | |
| NM_001206 | BTEB1 | SEQ ID NO: 138 | AAATGCACATTTACTTCCCAAAAAAGTTGTTACTT | SEQ ID NO: 139 |
| | | | GCCTTTTCAAGTGTGACAAACTCAC | |
| NM_003282 | TNNI2 | SEQ ID NO: 140 | CTCTGCAAACAGCTGCACGCCAAGATCGATGCGGC | SEQ ID NO: 141 |
| | | | TGAAGAGGAGAAGTACGACATGGAG | |
| NM_003943 | GENX- | SEQ ID NO: 142 | CTGGTCTGGATGTGATTAGTAAGGATACAAGATAA | SEQ ID NO: 143 |
| | 3414 | | TGTTCTAAGACTGCCTAATGCTTTT | |
| NM_000300 | PLA2G2A | SEQ ID NO: 144 | TTTGCTAGAAACAAGACGACCTACAATAAAAAGTA | SEQ ID NO: 145 |
| | | | CCAGTACTATTCCAATAAACACTGC | |
| NM_005139 | ANXA3 | SEQ ID NO: 146 | TTCAGCAATTAAATCGGATACTTCTGGAGACTATG | SEQ ID NO: 147 |
| | | | AAATCACACTCTTAAAAATCTGTGG | |
| NM_000111 | SLC26A3 | SEQ ID NO: 148 | TTTTGTAGCACTGACAGATTTCCATCCTAGTCACT | SEQ ID NO: 149 |
| | | | ACCTTCATGCATAGGTTTAGCAGTA | |
| NM_000353 | TAT | SEQ ID NO: 24 | TTTCTTAAAGTGAGAGCACGCCTGTACTAGAGCAA | SEQ ID NO: 150 |
| | | | GCAGGAATCAGAGACCTTCCAGAAA | |
| NM_004648 | PTPNS1 | SEQ ID NO: 151 | GCCTGTAAATTACTGTGAAATGTGAAACGTGCAAT | SEQ ID NO: 152 |
| | | | CTTGAAACTGAGGTGTTAGAAAACT | |
| NM_004778 | GPR44 | SEQ ID NO: 153 | GTCAGTGGAAGAAGCAGATGAGAAACTCTTGAGAT | SEQ ID NO: 154 |
| | | | CTTGGTCCTGTGTTTTTTCTGCCAC | |
| NM_003725 | RODH | SEQ ID NO: 155 | CTGGCATCATCAGAGTACTAACATGTTTATATTTC | SEQ ID NO: 156 |
| | | | AGATATCCAAAGCTTACCACTTTAG | |
| NM_020997 | LEFTB | SEQ ID NO: 157 | TCTTATTATTCACTGCACTATATTCTAAGCACTTA | SEQ ID NO: 158 |
| | | | CATGTGGAGATACTGTAACCTGAGG | |
| AB007969 | KIAA0500 | SEQ ID NO: 159 | CAGGGCTTAATTGACTATGTCTGAAAGTTTTTACT | SEQ ID NO: 160 |
| | | | GAGAGCTCTAAGAAAACTATTGAGG | |
| NM_001876 | CPT1A | SEQ ID NO: 161 | AATGACTGACATCATCACTTTGTTTGGTCTCAGTT | SEQ ID NO: 162 |
| | | | CTAATTCCAAAAAGTAATTCCACTG | |
| NM_000349 | STAR | SEQ ID NO: 163 | CGTGTTTAGAGTATGACACTAGGATTCAGATTGGT | SEQ ID NO: 164 |
| | | | GAAGTTTTTAGTACCAAGAAAACAG | |
| NM_004944 | DNASE1L3 | SEQ ID NO: 165 | CAAACGCTCCTAGACCCAAGGGTCTCATCTTATTA | SEQ ID NO: 166 |
| | | | ACCATTTCTTGCCTCTAAATAAAAT | |
| NM_004472 | FOXD1 | SEQ ID NO: 167 | CTCCCTTGACGTTTGGCAGATGAAAAACAACTAAG | SEQ ID NO: 168 |
| | | | CCTTTTTGAGGTGTAGAGATTCTCA | |
| NM_000735 | CGA | SEQ ID NO: 169 | TGAGATAAAACTCTCCTTTTCCTTACCATACCACT | SEQ ID NO: 170 |
| | | | TTGACACGCTTCAAGGATATACTGC | |
| NM_000777 | CYP3A5 | SEQ ID NO: 171 | CACAGATCCCCTTGAAATTAGACACGCAAGGACTT | SEQ ID NO: 172 |
| | | | CTTCAACCAGAAAAACCCATTGTTC | |
| NM_004839 | HOMER2 | SEQ ID NO: 12 | TCAACATCGAGAGGCTTTAAAACAACTGTTTAGTG | SEQ ID NO: 173 |
| NM_199330 | | SEQ ID NO: 13 | GAAACTTTCTGAGAGATGGAAAACA | |
| NM_199331 | | SEQ ID NO: 14 | | |
| NM_199332 | | SEQ ID NO: 15 | | |
| NM_016295 | GP2 | SEQ ID NO: 174 | ATTCATCTCTGTGATTCTCTTAATGAACAGTGCCA | SEQ ID NO: 175 |
| | | | GCCTTCTTGCTCAAGAAGTCAAGTC | |
| NM_018960 | GNMT | SEQ ID NO: 176 | TATAAGAGTGACTTGACCAAGGACGTCACAACATC | SEQ ID NO: 177 |
| | | | AGTGCTGATAGTGAACAACAAGGCC | |
| NM_004673 | ANGPTL1 | SEQ ID NO: 178 | ATTGATTGAATTAGAAGACTGGAGTGATAAAAAAG | SEQ ID NO: 179 |
| | | | TCTATGCAGAATACAGCAGCTTTCG | |
| NM_000765 | CYP3A7 | SEQ ID NO: 180 | GGATGAGACCGTAAGTGGAGCCTGATTTCCCTAAG | SEQ ID NO: 181 |
| | | | GACTTCTGGTTTGCTCTTTAAGAAA | |
| NM_002089 | CXCL2 | SEQ ID NO: 182 | TTCCCTCTGTGTTAGAGCAGAGAGGTTTCGATATT | SEQ ID NO: 183 |
| | | | TATTGATGTTTTCACAAAGAACAGG | |
| NM_006399 | BATF | SEQ ID NO: 184 | GCAGGCCCAATGCAGAAGAGTATTAAGAAAGATGC | SEQ ID NO: 185 |
| | | | TCAAGTCCCATGGCACAGAGCAAGG | |
| AK130329 | | SEQ ID NO: 186 | TATAAATTTGACTCTTAGTGTAAAATAATCTTAAT | SEQ ID NO: 187 |
| | | | ATTTGAATTATACATTCAGGTTATG | |
| NM_000376 | VDR | SEQ ID NO: 188 | TTGCCAGAAGTTTGTCAAGTCAACCAATGTAGAAA | SEQ ID NO: 189 |
| | | | GCTTTGCTTATGGTAATAAAAATGG | |
| NM_002727 | PRG1 | SEQ ID NO: 190 | AGTAGCTTGAGAAGCAAATTGGCAGGTAATATTTC | SEQ ID NO: 191 |
| | | | ATACCTAAATTAAGACTCTGACTTG | |

### Example 3

### Use of Cortisone Signature Genes to Evaluate HSD1-Agents

Gene expression changes in the cortisone signature genes were analyzed to further assess the degree to which HSD1-agents cause a reversal in the magnitude of the cortisone signature due to the degree of inhibition of HSD1 activity. Gene expression levels of the cortisone signature genes were measured in cortisone treated HepG2/HSD1 cells and HepG2/HSD1 cells treated with an HSD1-agent affecting HSD1 activity. In this example, replicate profiles were statistically averaged across the cortisone signature genes. Two gene expression vectors were then calculated: one representing the amplitudes of the cortisone signature genes in the cortisone treatment, and the other the amplitudes of the cortisone signature genes in the compound treatment. If a test compound treatment has a no HSD1 inhibition effect, and thus does not result in a significant change in the amplitude of the cortisone signature genes, then the linear regression between the amplitude vector of the cortisone treatment alone and the amplitude vector of the test agent will yield a slope of close to 1.0, which indicates that the test compound and the cortisone treatment alone profiles are statistically very similar, i.e., both profiles are from cells having very similar HSD1 activity. On the contrary, if the test compound treatment results in inhibition of HSD1 activity, then the slope of the linear regression between the cortisone treatment alone profile and the test compound will be smaller than 1.0. This measure of the inhibitory activity of an HSD1 compound is referred to as the “HSD1 signature reversal activity” of the compound.

Table 3 present data showing HSD1 signature reversal activity values measured for a variety of HSD1 inhibitor compounds measured from multiple experiments using HepG2/HSD1 cells incubated with 500 nM cortisone in the presence of 10 μM forskolin. The experimental conditions and microarray analysis was performed as set forth in Example 2 above, except that after 24 hours of cell culture, the Culture Medium was replaced with 500 μl Culture Medium containing 2× concentration of 11β-HSD1 inhibitor (14 μM) or vehicle for 20 min at 37° C./5% CO₂.

The HSD1 signature reversal activity values are presented in Table 3 for a series of test compounds and are sorted for convenience from less to more potent HSD1 inhibiting compounds. The cortisone slope to itself (defined as 1.0) is shown as control.

TABLE 3
Quantitative values for the strength of inhibition of
various HSD1 inhibitors using the cortisone
signature genes listed in Table 2.

| | HSD1 Signature Reversal |
|---|---|
| Compound Treatment | Activity Values |
| Cortisone/Forskolin (CF) | 1.0 |
| CF plus Compound 1 [415′] | 0.48 |
| CF plus Compound 2 [451′] | 0.22 |
| CF plus Compound 10 [400′] | 0.09 |
| CF plus Compound 3 [037′] | 0.07 |
| CF plus Compound 4 [544′] | 0.07 |
| CF plus Compound 5 [406′] | 0.05 |
| CF plus Compound 7 [750′] | 0.04 |
| CF plus 3-(2-chlorophenyl)-4-methyl-5-(4- | 0.03 |
| pentylbicyclo[2.2.2]oct-1-yl)-4H-1,2,4-triazole | |
| CE plus Compound 6 [698′] | 0.03 |

Table 4 shows an experiment where HSD1 activity of samples treated with some of the compounds evaluated in Table 3, were measured directly. HSD1 activity was measured by spiking the incubation medium with ³H-cortisone. After 21 hrs post compound treatment, conversion of the cortisone to cortisol was assessed by HPLC analysis.

TABLE 4
Direct measurement of HSD1 activity.

| | Percent Conversion |
|---|---|
| | to Cortisol |
| | (normalized to |
| Compound Treatment | cortisone/forskolin value) |
| Cortisone/Forskolin (CF) | 40 [1.0] |
| CF plus Compound 1 | 12 [0.3] |
| CF plus Compound 4 | 5 [0.12] |
| CF plus Compound 2 | 4 [0.1] |
| CF plus 3-(2-chlorophenyl)-4-methyl-5-(4- | 2 [0.05] |
| pentylbicyclo[2.2.2]oct-1-yl)-4H-1,2,4-triazole | |
| CF plus Compound 3 | 1 [0.025] |
| CF plus Compound 6 | 0.5 [0.0125] |
| CE plus Compound 5 | 0 |

A comparison of the data trends in Tables 3 and 4 demonstrate that the degree of reversal of the cortisone signature gene expression values measured for the HSD1 inhibitor compounds (Table 3) is similar to the amount of inhibition of HSD1 activity measured for each of the HSD1 compounds (Table 4).

### Example 4

### Evaluation of Off-Target Effect of HSD1 Compounds Using Microarrays

HSD1 compounds were evaluated using microarray gene expression measurements obtained from HepG2/HSD1 cells under conditions where the cells were not treated with cortisone. Transcription levels were compared between samples of cells treated for 20 hours with an HSD1 inhibitor compound plus forskolin and cells treated for 20 hours with forskolin alone. The off-target activity of each HSD1 inhibitor compound was estimated by determining the number of genes represented on the microarray exhibiting at least a 1.5-fold change, with a p-value of <0.01, in transcription level between the HSD1 compound treated cells compared to the control, forskolin, no HSD1, cell sample.

Table 5 shows the use of this method to rank-order a set of HSD1 inhibitor compounds.

TABLE 5
Evaluation of off-target affects of HSD1 compounds.

| | Number of |
|---|---|
| | Off-Target Genes |
| | (>1.5-fold change, |
| Compound Treatment | p-value <0.01) |
| 10 μM Forskolin alone plus Compound 2 | 627 |
| 10 μM Forskolin alone plus 3-(2-chlorophenyl)-4- | 394 |
| methyl-5-(4-pentylbicyclo[2.2.2]oct-1-yl)-4H- | |
| 1,2,4-triazole | |
| 10 μM Forskolin alone plus Compound 5 | 121 |
| 10 μM Forskolin alone plus Compound 6 | 96 |
| 10 μM Forskolin alone plus Compound 1 | 81 |
| 10 μM Forskolin alone plus Compound 3 | 58 |
| 10 μM Forskolin alone plus Compound 4 | 36 |
| 10 μM Forskolin alone plus Compound 7 | 35 |
| 10 μM Forskolin alone plus Compound 10 | 17 |

Based upon the data in Table 5, Compound 2 and the compound 3-(2-chlorophenyl)-4-methyl-5-(4-pentylbicyclo[2.2.2]oct-1-yl)-4H-1,2,4-triazole were identified as HSD1 inhibitors exhibiting high levels of off-target activity as indicated by the large number of genes exhibiting a significant change in expression changes compared to cells treated with forskolin alone. In contrast, HSD 1 inhibitor Compound 10 was identified as having low off-target activity because only 17 genes of the approximately 24,000 genes represented on the microarrays used in these experiments were recorded as having significant changes in expression level compared to forskolin only treated cells.

Joint consideration of the data in Tables 3 and Table 5 demonstrates the power of evaluating HSD1 inhibitory agents based using both the strength of the reversal of the cortisone signature genes, that is the degree of on target inhibition of HSD1, and the extent of HSD1 off-target effects on gene expression. For example, from inspection of Table 3 and Table 5 it is evident that Compounds 4 and 7 have the therapeutically desirable properties of relatively high inhibition of HSD1 activity and a relatively low level of off-target effects. Thus, Compounds 4 and 7 are relatively good candidate therapeutic compounds as compared to the other compounds tested.

### Example 5

### Identification of an Off-Target Response Signature

The off-target signature of HSD1 inhibitor compound 3-(2-chlorophenyl)-4-methyl-5-(4-pentylbicyclo[2.2.2]oct-1-yl)-4H-1,2,4-triazole was identified from experiments that were performed using cells treated with 3-(2-chlorophenyl)-4-methyl-5-(4-pentylbicyclo[2.2.2]oct-1-yl)-4H-1,2,4-triazole and foroskolin. Microarray experiments were conducted using the methods described in Example 2. In this study the off-target expression signature was computed by combining replicates of the 3-(2-chlorophenyl)-4-methyl-5-(4-pentylbicyclo[2.2.2]oct-1-yl)-4H-1,2,4-triazole+foroskolin samples in an error weighted fashion using an error model developed for two-color hybridization experiments (see Parrish et al., 2004 J. Neurosci. Methods, 132:57-68) and selecting those genes having expression level difference with a p-value<0.01 (no fold expression change cut-off). This procedure was carried out independently for the three replicate studies and then an off-target response signature of 330 gene markers was identified (Table 6) that was common to each of the three data sets.

TABLE 6
HSD1 off-target gene markers.

| Reference | | | | |
|---|---|---|---|---|
| Transcript | Gene Name | SEQ ID NO: | Probe Sequence | SEQ ID NO: |
| NM_005063 | SCD | SEQ ID NO: 30 | AATAATGCTACCAGGATGCTAAAGATGATGA | SEQ ID NO: 192 |
| | | | TGTTAACCCATTCCAGTACAGTATTCTTT | |
| Contig28780_RC | | SEQ ID NO: 193 | GGTTAATGAGACAAGTATGATCCAACTTGTG | SEQ ID NO: 194 |
| | | | TTTGCAATTAAGCAGGGATACAGGTACTG | |
| NM_018254 | RCOR3 | SEQ ID NO: 195 | CATAATATTGCGTTTGTGTCAGACATTGGAT | SEQ ID NO: 196 |
| | | | TTCTATGTTAGATATTAAAGAGCTTACTT | |
| NM_005950 | MT1G | SEQ ID NO: 197 | CTCCCAAGTACAAATAGAGTGACCCGTAAAA | SEQ ID NO: 198 |
| | | | TCTAGGATTTTTTGTTTTTTGCTACAATC | |
| NM_005951 | MT1H | SEQ ID NO: 199 | TGATGTCGGGACAGCCCTGCTGTCAGATGAA | SEQ ID NO: 200 |
| | | | AACAGAATGACACGTAAAATCCGAGGTTT | |
| NM_014443 | IL17B | SEQ ID NO: 201 | CTTGCACCTTTGTGCCAAGAAAGGCCTATGA | SEQ ID NO: 202 |
| | | | AAAGTAAACACTGACTTTTGAAAGCCAGA | |
| AK131274 | | SEQ ID NO: 203 | CTGCCCGAACATAACCATATTGCAGATGAAA | SEQ ID NO: 204 |
| | | | ATTTTCAATAGAGAATGACACAATTCTTG | |
| NM_001394 | DUSP4 | SEQ ID NO: 205 | AAAGAAATCTTCGAAGGTGTGGTTTTCATTT | SEQ ID NO: 206 |
| | | | CTCAGTCACCAACAGATGAATAATTATGC | |
| BM696623 | | SEQ ID NO: 207 | AATTCTTTTTTAAATTGGTATATTGTAAGAT | SEQ ID NO: 208 |
| | | | ATCTATGCAAAAAATGTTATGTGACGCAT | |
| NM_005763 | AASS | SEQ ID NO: 96 | AGGCAATACACCTCTGAACATGTGTGTGATA | SEQ ID NO: 97 |
| | | | AATGGGTTTGCTAATGTGCTGTTTTAAAG | |
| NM_016364 | DUSP13 | SEQ ID NO: 209 | CCTGACCCAATTCAGAGATTCTTTATGCAAA | SEQ ID NO: 210 |
| | | | AGTGAGTTCAGTCCATCTCTATAATAAAA | |
| NM_001096 | ACLY | SEQ ID NO: 32 | GGAGGAAGGGAATGAAACATTTATGGCCTCA | SEQ ID NO: 211 |
| NM_198830 | | SEQ ID NO: 33 | AGATACTATACATTTAAAGCACCCCAATG | |
| NM_005891 | ACAT2 | SEQ ID NO: 212 | CCAAGTTTACAGCTTGTACTTTACTTTAATG | SEQ ID NO: 213 |
| | | | TGTAATACTCAACTCACGGTACAAGACAA | |
| NM_018677 | ACAS2 | SEQ ID NO: 214 | GAACGCTTTGAGACAACCTACTTTAAGAAGT | SEQ ID NO: 215 |
| | | | TTCCTGGATACTATGTTACAGGAGATGGC | |
| NM_000434 | NEU1 | SEQ ID NO: 216 | GTCCTCTCCCACAAAAAAGTCCTGCCCTCAT | SEQ ID NO: 217 |
| | | | CTGAGAATACTGTCTTTCCATATGGCTAA | |
| NM_002004 | FDPS | SEQ ID NO: 218 | GGATCTTGTCAGATTCACTGAAAAGAGGTAC | SEQ ID NO: 219 |
| | | | AAATCTATTGTCAAGTACAAGACAGCTTT | |
| NM_004265 | FADS2 | SEQ ID NO: 220 | ACCCATAGGGAGCTGATCGTAATGTTTATCA | SEQ ID NO: 221 |
| | | | TGTTACTTCCCCACCCCTACATTTTTTGA | |
| NM_001360 | DHCR7 | SEQ ID NO: 28 | CCTCATCCCTAGGGTGTTGTGTAGAACTCTT | SEQ ID NO: 222 |
| | | | TTTAAACTCTATGCTCCGAGTAGAGTTCA | |
| NM_013262 | MYLIP | SEQ ID NO: 223 | CCAACGCACACCAGTCTTCTCAATCTGACTG | SEQ ID NO: 224 |
| | | | TAATCTAATCTGTTGTGCTTTTGTTGGAC | |
| AB040944 | ZSWIM5 | SEQ ID NO: 225 | CTTTCTTCGACAAACTGTCACCTTTTTTACT | SEQ ID NO: 226 |
| | | | CCAAACTGACCAAGAAGTTGTATTTGCCA | |
| NM_002304 | LFNG | SEQ ID NO: 227 | TTGTTTTTCTCTTTGCAAAGACATAGCTAGG | SEQ ID NO: 228 |
| | | | AAAGCGAATGATAAGGGAAAAGTTCTCAG | |
| NM_016075 | C13orf9 | SEQ ID NO: 229 | CCAGGGCTTTTAGAAGCAGTCATAGACATGT | SEQ ID NO: 230 |
| | | | CTTCAACATACCAAATAAAATACCTTTAA | |
| NM_004289 | NFE2L3 | SEQ ID NO: 231 | TTCATAGTTATGTCCAAAGAATAGGTTAACA | SEQ ID NO: 232 |
| | | | TGAAAACCCAGTAAGACTTTCCATCTTGG | |
| NM_004278 | PIGL | SEQ ID NO: 233 | TAGCTACACTGCTAGCTTCTCAAGTTCTTGT | SEQ ID NO: 234 |
| | | | GAAAAACAATTTACATAATGACACAGTAG | |
| NM_016348 | C5orf4 | SEQ ID NO: 235 | ATCAAACAAAATCCTTGAGGTTGGTATACAA | SEQ ID NO: 236 |
| | | | GTTAAGGCTGAAAAAAGGCCTTAAATTCC | |
| NM_006469 | IVNS1ABP | SEQ ID NO: 237 | AACTGTAGGGAACACCATTTATGCAGTGGAG | SEQ ID NO: 238 |
| | | | GATTCGATGGCAATGAATTTCTGAATACG | |
| NM_001486 | GCKR | SEQ ID NO: 239 | TGCTCTCGACCTAGTGGTTTCTACTCTCACC | SEQ ID NO: 240 |
| | | | GACTTATTCTGATTTCAGAAATAAAATGA | |
| AB033025 | KIAA1199 | SEQ ID NO: 100 | CATATGTTTCACAGTACAGGATCTGTACATA | SEQ ID NO: 101 |
| | | | AAAGTTTCTTTCCTAAACCATTCACCAAG | |
| NM_015986 | CRLF3 | SEQ ID NO: 241 | AGTAGATTTCCACTTGTTTAGGGATTAAGAA | SEQ ID NO: 242 |
| | | | GATGTTCTAGAATGTAGATACCTTGTCCA | |
| NM_198581 | ZC3H6 | SEQ ID NO: 243 | TAAGTGGAAGGAGAGTTACTGAAGGGAATGT | SEQ ID NO: 244 |
| | | | GAATTTTTACCGTTTGTACTTAAGATACA | |
| NM_007267 | EVER1 | SEQ ID NO: 245 | GGTGAGGACAAAATCTTCTTAATCAACAAGC | SEQ ID NO: 246 |
| | | | TTCACTCCATCTACGAGAGGAAGGAGAGG | |
| NM_000353 | TAT | SEQ ID NO: 24 | TTTCTTAAAGTGAGAGCACGCCTGTACTAGA | SEQ ID NO: 150 |
| | | | GCAAGCAGGAATCAGAGACCTTCCAGAAA | |
| NM_021184 | C6orf47 | SEQ ID NO: 247 | TAAATAACTGTGTCACATCAAATCCTAAATA | SEQ ID NO: 248 |
| | | | TACCACTACAAAGTGAGAGTTACTGCCAC | |
| AK000954 | | SEQ ID NO: 249 | TTTAAACACCTTAAGAAGGAACGAAAAAGTT | SEQ ID NO: 250 |
| | | | CTCTTGGATGACGTATATTCAAGTCTGAG | |
| NM_002053 | GBP1 | SEQ ID NO: 251 | TGGGCTAATGAAGAAAACTTCTCTTATTGTT | SEQ ID NO: 252 |
| | | | CAGTTCATCCAGATTATAACTTCAATGGG | |
| NM_018024 | FLJ10204 | SEQ ID NO: 253 | GCTGAATCAGCAATAAGTATTAGTCTTTTTG | SEQ ID NO: 254 |
| | | | GACTATGGTATTGTTAAAAAGACTGCAGC | |
| NM_004998 | MYO1E | SEQ ID NO: 255 | GTACTAAGTAGTATTCCACTGTACTCATTCA | SEQ ID NO: 256 |
| | | | TAAGGTAGGTTTTCTTACAAAACTCACAC | |
| BX537667 | | SEQ ID NO: 257 | TACCTTGAAACAACTGCAGTTCTAACTCATT | SEQ ID NO: 258 |
| | | | GTCACCATTTCTGTTTGACACGTTCCACT | |
| NM_004860 | FXR2 | SEQ ID NO: 259 | CCTTGTCTGGTGGATGAGAAGGCGTATTTAT | SEQ ID NO: 260 |
| | | | TTTTCACTGTACAGTATTTAAAAAGAGAA | |
| NM_006350 | FST | SEQ ID NO: 261 | CTGCCCGTAAAACCTGAGCCATTGATTCTTC | SEQ ID NO: 262 |
| | | | AGAACTTTCTGCAGTTTTTGACTTCATAG | |
| NM_005588 | MEP1A | SEQ ID NO: 263 | CATCACTCTTTTAAGATCTTGTTAAAGATTT | SEQ ID NO: 264 |
| | | | CAAATAAAGGTACTTCTGGCGAGCCAGGC | |
| NM_020371 | AVEN | SEQ ID NO: 265 | TTTACAAGCATGTCTCAAGCTAGTGTGTTCC | SEQ ID NO: 266 |
| | | | ATTATGCTCACAGCAGTAAATGCCTACCT | |
| NM_000368 | TSC1 | SEQ ID NO: 267 | CGGACAGTGTTGGACAGCTACATATCATGGA | SEQ ID NO: 268 |
| | | | CTACAATGAGACTCATCATGAACACAGCT | |
| NM_012448 | STAT5B | SEQ ID NO: 269 | CACCAGAGGAATCACTCTTGTGGATGTTTTA | SEQ ID NO: 270 |
| | | | ATTCCATGAATCGCTTCTCTTTTGAAACA | |
| NM_018371 | ChGn | SEQ ID NO: 271 | CAGAAGTAGGGACCGCTTTCTTACCTGTTTA | SEQ ID NO: 272 |
| | | | AATAAACCAAAGTATACCGTGTGAACCAA | |
| NM_001037 | SCN1B | SEQ ID NO: 273 | CCCATTCAGCCTCGTCTCTTTACAGAAGTAG | SEQ ID NO: 274 |
| | | | TTTTGTTCATGAAATAAAGACTCTTGGAC | |
| NM_000392 | ABCC2 | SEQ ID NO: 275 | AGTGTGTATAAAATGTACGTTTTAAAAAAGG | SEQ ID NO: 276 |
| | | | ATAAGTGAACACCCATGAACCTACTACCC | |
| NM_017566 | KLHDC4 | SEQ ID NO: 277 | AGGTGGATTCCCTCAGCAGCCTGTGTGTTGT | SEQ ID NO: 278 |
| | | | GTGAAATAAATTGGACTTGAAACAAAAAA | |
| NM_013261 | PPARGC1A | SEQ ID NO: 111 | CTGTAGTCTAAGACCTGATCTATAGATACCT | SEQ ID NO: 112 |
| | | | AGAATAGCCATGTACTATAATGTGATGAT | |
| NM_012296 | GAB2 | SEQ ID NO: 279 | ACTTGTGTCTTGAAATCTAGAATTATTTCAC | SEQ ID NO: 280 |
| | | | GCAGAATTGTCACTGTTTGTCAGGAAGAG | |
| Contig15566_RC | KLF7 | SEQ ID NO: 281 | GGATTATATTTGAATGTGTGGTGCATCCTTC | SEQ ID NO: 282 |
| | | | CTGGATGAAGGATGTGTGAGGGACCTTGA | |
| BG546127 | | SEQ ID NO: 283 | TCTGTCAGCATTTTTTAACTAAAGGTCACAA | SEQ ID NO: 284 |
| | | | CCCATTAACAACCATGAAATTGGTGTTGG | |
| NM_006703 | NUDT3 | SEQ ID NO: 285 | TGAGATGAAAACTTCCAATAATTTGTCCTAT | SEQ ID NO: 286 |
| | | | AATGTGCTGTACAGTTCAGTAGAGTGGTC | |
| NM_021090 | MTMR3 | SEQ ID NO: 287 | ATTAATATTTTCCTTTGTTAAAGGAGGAACC | SEQ ID NO: 288 |
| | | | GTAACTCTCCATAGCTGTACATATAACCC | |
| NM_001621 | AHR | SEQ ID NO: 289 | AATGGCTTCGGACAAAATATCTCTGAGTTCT | SEQ ID NO: 290 |
| | | | GTGTATTTTCAGTCAAAACTTTAAACCTG | |
| NM_002583 | PAWR | SEQ ID NO: 291 | TACATGAGTTCCAATACTTTTGGGATGTTAC | SEQ ID NO: 292 |
| | | | CCTCACATGTCCCTATACTGATGTGTGCC | |
| NM_002342 | LTBR | SEQ ID NO: 293 | ATTTATATTGTAACCACATGCAAATAAAAAG | SEQ ID NO: 294 |
| | | | AATGGGACCTAAACTCGTGCCGCTCGTGC | |
| NM_013974 | DDAH2 | SEQ ID NO: 295 | GATCCACTGGGTGAATCCTCCCTCTCAGAAC | SEQ ID NO: 296 |
| | | | CAATAAAATAGAATTGACCTTTTAAAAAA | |
| NM_003904 | ZNF259 | SEQ ID NO: 297 | TTTGAGTCAGAGATCTGTGCACACTTTTTAA | SEQ ID NO: 298 |
| | | | ACAGCTTGTGATGCAAGTGTGAGCCTATT | |
| AL133047 | DKFZp434D0215 | SEQ ID NO: 299 | GGACTATGGGGACTTCTAATACGTTTTGGTA | SEQ ID NO: 300 |
| | | | AAGAAGAGAGTATAAAGAAAATTCTTGTC | |
| NM_003373 | VCL | SEQ ID NO: 301 | ACTAAAGAAAGATTACTTAGAGGAAATAAGA | SEQ ID NO: 302 |
| | | | AAAATCATGTTTGCTCTCCCGGTTCTTCC | |
| NM_000663 | ABAT | SEQ ID NO: 303 | CACCTGTTCCTCAATATTTTCAGTGACATCT | SEQ ID NO: 304 |
| | | | TAGCAGACTTCAAGTAAAGAAGCCATTTC | |
| NM_003826 | NAPG | SEQ ID NO: 305 | ATAAGAAAACAATTGCTCAAGTCTTAGTTCA | SEQ ID NO: 306 |
| | | | TCTACACAGAAATGACTATGTAGCTGCAG | |
| NM_013370 | OKL38 | SEQ ID NO: 307 | GGACTTAGACCAGTGTGTGAGGTTGGACTTA | SEQ ID NO: 308 |
| | | | GACCAGTGTGTGAGGTGGTAACAGCGGCC | |
| NM_014000 | VCL | SEQ ID NO: 309 | CTGTGTGTTAGTTCCCAACATCGAATGTGTA | SEQ ID NO: 310 |
| | | | CAACTTAAGTTGGTCCTTTACACTCAGGC | |
| NM_018676 | THSD1 | SEQ ID NO: 311 | TTTTGTGTAACTATTTGTACCGTAGGACAGA | SEQ ID NO: 312 |
| | | | ATGTGAGGAGGAAGTAACACACAGAGGAG | |
| NM_016531 | KLF3 | SEQ ID NO: 313 | CTGAATTTATATCATCCAAAACTTCCATATG | SEQ ID NO: 314 |
| | | | GTCAGTAGTAGATGTTCTCTAATCCTCCC | |
| NM_004664 | LIN7A | SEQ ID NO: 315 | TGAGGGAAAGCTACTTGATCAAACATCCGAT | SEQ ID NO: 316 |
| | | | AGTCACAAATTTGAAACCGTGCTTCAGAA | |
| NM_015516 | E2IG4 | SEQ ID NO: 317 | GGGATGGAGACATGTCATTTGTAAAAGCAGA | SEQ ID NO: 318 |
| | | | AAAAGGTTGCATTTGTTCACTTTTGTAAT | |
| NM_004490 | GRB14 | SEQ ID NO: 319 | GGACAAAAAATAAAGCACTTTCAAATTATAC | SEQ ID NO: 320 |
| | | | CAGTAGAAGATGACGGTGAAATGTTCCAC | |
| AB028949 | KIAA1026 | SEQ ID NO: 321 | CTCTGTAATTGATGTACATACCGCAAACCGT | SEQ ID NO: 322 |
| | | | GTGTGAACCTGTCAACTCTCTGTCGTCTT | |
| NM_000574 | DAF | SEQ ID NO: 323 | AAAGCCTGTGAAAGGTGTCTTCTTTGACTTA | SEQ ID NO: 324 |
| | | | ATGTCTTTAAAAGTATCCAGAGATACTAC | |
| NM_005446 | P2RXL1 | SEQ ID NO: 325 | ACCTACTCTTTAGAGAGCCCCAGCATCTTTG | SEQ ID NO: 326 |
| | | | ATGTGGATTGGAGACAATTGCCTGGTTCC | |
| NM_003363 | USP4 | SEQ ID NO: 327 | ACTGCATATGCGAAGAACAAACTGAATGGTA | SEQ ID NO: 328 |
| | | | AATGGTATTACTTTGATGATAGCAACGTG | |
| NM_000782 | CYP24A1 | SEQ ID NO: 329 | GATTTCCCATGCCATAATTTTTCTGTCTATT | SEQ ID NO: 330 |
| | | | AAATGGGACAAGTGTAAAGCATGCAAAAG | |
| NM_014660 | PHF14 | SEQ ID NO: 331 | ATATGAACTTAGGAAATAAGGAGGGAAGTAC | SEQ ID NO: 332 |
| | | | AAAGATTATTGACTATACAACCTACCAGC | |
| NM_207343 | DKFZp547C195 | SEQ ID NO: 333 | GTCTGTATTCCAATGTTCGTAAATGAAACTA | SEQ ID NO: 334 |
| | | | TGTATATTATGCAGAAACAGTCTGTTCCC | |
| NM_012258 | HEY1 | SEQ ID NO: 42 | GAGTGGTCAATTGAGTAGTACAGTGGAAACT | SEQ ID NO: 335 |
| | | | GTTAAATGCATAACCTAATTTTCCTGGGA | |
| NM_003508 | FZD9 | SEQ ID NO: 336 | AATGATGTAATTTATTGTTGCGTTCCTCTGG | SEQ ID NO: 337 |
| | | | AAGCTGTGACTGGAATAAACCCCCGCGTG | |
| AB007932 | PLXNA2 | SEQ ID NO: 338 | GTGACTGCTGCTTTGCATGAAAACTCATTTG | SEQ ID NO: 339 |
| | | | ATGTATATTGGGGAAATAATGAGAACTTT | |
| NM_000874 | IFNAR2 | SEQ ID NO: 340 | GTGGAAATTTCACCTATATCATTGACAAGTT | SEQ ID NO: 341 |
| | | | AATTCCAAACACGAACTACTGTGTATCTG | |
| NM_018223 | CHFR | SEQ ID NO: 342 | GCTTCTTCTCAGGAATACAGTTTTCAACTGT | SEQ ID NO: 343 |
| | | | TGTCTTGCTCTTGATAGAAACTGAGAAGC | |
| NM_016446 | C9orf127 | SEQ ID NO: 344 | CTTCCCAAACTGTCATGCATAGATAATGGTC | SEQ ID NO: 345 |
| | | | ATTTTTGTAAGACACATTGGAGTAAATGA | |
| NM_006281 | STK3 | SEQ ID NO: 346 | AATTGGTAAACATCATGTTCCTGATGATAAC | SEQ ID NO: 347 |
| | | | CCAGTAGCAAAAACATTTGTACTGAGTGG | |
| NM_017762 | FLJ20313 | SEQ ID NO: 348 | TGTTGTTCAACAGTATTGAGTGTGAAGGAAA | SEQ ID NO: 349 |
| | | | TTTTGATGGCTTAATAAAATTCCACAGCG | |
| NM_017911 | C22orf8 | SEQ ID NO: 350 | ATCTGACAGGCTCTCTTTTGTCAAGGTGGTA | SEQ ID NO: 351 |
| | | | TTTTTCGTAATAAAAGGGGAAGAGTAAAG | |
| NM_018075 | FLJ10375 | SEQ ID NO: 352 | CATTAACAGACCCTTTATCAACATAAACAAT | SEQ ID NO: 353 |
| | | | AGTAACTGAGCTATTAAAGGCAACCTCTC | |
| NM_003224 | ARFRP1 | SEQ ID NO: 354 | TGTTTTCTCGAAGACAAACTTTCCTCTATGT | SEQ ID NO: 355 |
| | | | CTGGAAAAGCGTAGGCATCCGGAGGCTTT | |
| NM_017458 | MVP | SEQ ID NO: 356 | CAACCACGTGGTGCCTGTACTGCGCTAACTC | SEQ ID NO: 357 |
| | | | CTGATTAATACAATGGAAGTTTCTGGGCA | |
| NM_002662 | PLD1 | SEQ ID NO: 358 | CATTGATGTAAGGACTGTAAACATCAGCAAG | SEQ ID NO: 359 |
| | | | ACTTTATAATTCCTTCTGCCTAACTTGTA | |
| NM_005053 | RAD23A | SEQ ID NO: 360 | ACCCCAGAACAGAACCGTGTCTCTGATAAAG | SEQ ID NO: 361 |
| | | | GTTTTGAAGTGAATAAAGTTTTAAAAACT | |
| AK022225 | | SEQ ID NO: 362 | GGTACATAATGAATGCCTTTGGTACTCATTT | SEQ ID NO: 363 |
| | | | TCTGAATGGTTTGCTCTAGTAGGAAAGAA | |
| AL080218 | STAT5B | SEQ ID NO: 364 | ATCCTATGCAGAGTTACAGTCACAAAGTTGT | SEQ ID NO: 365 |
| | | | GTATTTTATGTTACAATAAAGCCTTCCTC | |
| NM_014028 | OSTM1 | SEQ ID NO: 366 | CTTTAGACCTAGCATCAGCAATAGATTTCAA | SEQ ID NO: 367 |
| | | | AGATAAGTATTAAGCGCTACCCTAAAGTG | |
| NM_002061 | GCLM | SEQ ID NO: 368 | CTGACTTAGGAGCATAACTTACCTGTAATTT | SEQ ID NO: 369 |
| | | | CCTTCAATATGAGAGAAAATTGAGATGTG | |
| NM_012227 | PGPL | SEQ ID NO: 370 | TGAACTGCTTTCCCTCGGAATGTTTCCGTAA | SEQ ID NO: 371 |
| | | | CAGGACATTAAACCTTTGATTTTAAAAAA | |
| NM_005908 | MANBA | SEQ ID NO: 372 | TGGCGATTGAAATGTTACAACAGAGATTTCT | SEQ ID NO: 373 |
| | | | TGGTGGTAGCTATTTGGGTAAAGGTATAT | |
| NM_014448 | ARHGEF16 | SEQ ID NO: 374 | GGACCCGGCAGGGAGATTTCGGTTTTGAGGT | SEQ ID NO: 375 |
| | | | TTCTAAATACATTAAAGTTATTTCTTAAG | |
| NM_012237 | SIRT2 | SEQ ID NO: 376 | ATGCTGGATGGGCAGAAGACATTGCTTATTG | SEQ ID NO: 377 |
| | | | GAGACAAATTAAAAACAAAAACAACTAAC | |
| AF131763 | MGC11349 | SEQ ID NO: 378 | CTGTCCTTCCTAAAAATGGAATAGTGGGATG | SEQ ID NO: 379 |
| | | | TAGTGCTTAATGGAAACTGCTAAATCTTT | |
| NM_018231 | FLJ10815 | SEQ ID NO: 380 | CACTGTTTCTTTTCTACTTGTGTATACTCGA | SEQ ID NO: 381 |
| | | | GAAAGGGAAGATGGTTAATAAAAGGGATT | |
| NM_014630 | ZNF592 | SEQ ID NO: 382 | CTGAGAACTAATTGTCACAGACATTGGAATA | SEQ ID NO: 383 |
| | | | TTTGTACTGCTCTCGTGCCATTTGAGAGG | |
| NM_012127 | CIZ1 | SEQ ID NO: 384 | CTGCCTGGAAACTTTGACATGGTTCATGTTT | SEQ ID NO: 385 |
| | | | TTACTCAAAATCCAATAAAACAAGGTAGT | |
| NM_012342 | BAMBI | SEQ ID NO: 386 | ATGACCTCTGCAAACAGAATCTTGGATATTT | SEQ ID NO: 387 |
| | | | CTTCTGAAGGATTATTTGCACAGACTTAA | |
| NM_014030 | GIT1 | SEQ ID NO: 388 | GTGGGTGGGCTGTCAAGACGTGTCATGTACA | SEQ ID NO: 389 |
| | | | TTTGTATCAAAAATAAAGAAGTGACCATG | |
| AB018310 | KIAA0767 | SEQ ID NO: 390 | GTACATCTGGGCAGATGTTTAATTTCTGTGA | SEQ ID NO: 391 |
| | | | CTAATCACTGAACTAGACGAATGTTAAAT | |
| NM_002957 | RXRA | SEQ ID NO: 392 | CACTGCTCTGCCTAAGAGATGTGTTGTCACC | SEQ ID NO: 393 |
| | | | CTCCTTATTTCTGTTACTACTTGTCTGTG | |
| NM_015675 | GADD45B | SEQ ID NO: 394 | TACCCATGAACTCCCAGTTTGCGAATTAATA | SEQ ID NO: 395 |
| | | | AGAGACAATCTATTTTGTTACTTGCACTT | |
| NM_001967 | EIF4A2 | SEQ ID NO: 396 | GGTTATAAATTATGATCTACCTACCAATCGT | SEQ ID NO: 397 |
| | | | GAAAACTATATTCACAGAATTGGCAGAGG | |
| NM_014325 | CORO1C | SEQ ID NO: 398 | TTTGATGACTGTACAACAGGAAGACTTGAAA | SEQ ID NO: 399 |
| | | | AATCACGTGGATTCATATTACCACCGCTC | |
| NM_004565 | PEX14 | SEQ ID NO: 400 | GACCTCAGAACGGAAGATAGGACTGTATATA | SEQ ID NO: 401 |
| | | | ATTGTAATAAATACCAGTTGCCACTATTT | |
| NM_005165 | ALDOC | SEQ ID NO: 402 | GAGGGTAGTACAGAAGCCCTGACTCATGCCT | SEQ ID NO: 403 |
| | | | TGAGTACATACCATACAGCAAATAAATGG | |
| NM_017836 | SLC41A3 | SEQ ID NO: 404 | ATGGGGCCTCATACAACCCTTCATCTGCACT | SEQ ID NO: 405 |
| | | | CAACATTTAATCGTGTCCTTGCTGTCTTT | |
| NM_017712 | PGPEP1 | SEQ ID NO: 406 | TTTAAAGCACTGTGTGACATAGCTCCTTAGA | SEQ ID NO: 407 |
| | | | GATATAACCTATTGTCTGCTCATTGTCAA | |
| NM_018182 | FLJ10700 | SEQ ID NO: 408 | AATACCTGGATTCCCCATTCTATCCACATAC | SEQ ID NO: 409 |
| | | | TGTAGCATTCATTCTCCATCTCTCTCACC | |
| NM_001292 | CLK3 | SEQ ID NO: 410 | GTGCCCAGCTGCCAGAAAGCACAGATTTGAC | SEQ ID NO: 411 |
| | | | CCAAGCTATTTATATGTTATAAAGTTATA | |
| NM_014719 | KIAA0738 | SEQ ID NO: 412 | ATAAGACTGCTAAAAGTATTTGGTATACAGT | SEQ ID NO: 413 |
| | | | CTGGAAAATAAAGTTGAGGGAATCTCTCC | |
| AB014538 | PHLDB1 | SEQ ID NO: 414 | CTGGGGGTTTTCAGTATTTGTAAGCATTTCA | SEQ ID NO: 415 |
| | | | GCAGAACAATAAAGCCTTTGGACTACGGA | |
| NM_003659 | AGPS | SEQ ID NO: 416 | TGGAGAAGATAATGGACAGAGAGGTTATTTG | SEQ ID NO: 417 |
| | | | CTGACCTATGTTATTGCATACATTCGAGA | |
| NM_002461 | MVD | SEQ ID NO: 418 | TGAAGGGCTTTCTCACCCCAGCTCTGGCTAT | SEQ ID NO: 419 |
| | | | GCCCAGTTCTCTGAGAAAGGAGCTCAGTG | |
| NM_001892 | CSNK1A1 | SEQ ID NO: 420 | TCGCCGAGATGACATGGAATCATTAGGATAT | SEQ ID NO: 421 |
| | | | GTTTTGATGTATTTTAATAGAACCAGCCT | |
| NM_017855 | APIN | SEQ ID NO: 422 | ACAGCCTAAGGGAACCATAAGAAGTTGCCCT | SEQ ID NO: 423 |
| | | | GATCATTCAGACATTTTGGGAAAAAGATG | |
| NM_018383 | WDR33 | SEQ ID NO: 424 | GAAATGGATGAGAAACATTGTATGTAGGGAT | SEQ ID NO: 425 |
| | | | GTTTAGCAATCAGTCTTTTAATAGACAGC | |
| AL117568 | | SEQ ID NO: 426 | TAGTGATGATAAATTCTGTAGGAGGGTCTAT | SEQ ID NO: 427 |
| | | | TCTGAGCCGTTAACTTCCTGTAAGGGGAA | |
| NM_014176 | HSPC150 | SEQ ID NO: 428 | CACTCCAATTTATCATCCAAACATTGATTCT | SEQ ID NO: 429 |
| | | | GCTGGAAGGATTTGTCTGGATGTTCTCAA | |
| NM_005662 | VDAC3 | SEQ ID NO: 430 | CTTCGACCAGGAGTCAAATTGACTTTATCAG | SEQ ID NO: 431 |
| | | | CTTTAATCGATGGGAAGAACTTCAGTGCA | |
| AK094351 | | SEQ ID NO: 432 | CCTGAACTTTGGGAGTTTTAAGCAATGATAC | SEQ ID NO: 433 |
| | | | AGTTGAAGGGACTGTTTATAATTTTCACA | |
| NM_004039 | ANXA2 | SEQ ID NO: 434 | TCCCGCAGTGAAGTGGACATGTTGAAAATTA | SEQ ID NO: 435 |
| | | | GGTCTGAATTCAAGAGAAAGTACGGCAAG | |
| NM_003983 | FLJ13291 | SEQ ID NO: 436 | TTAAAGAAGGAATCACTTTCCTATCATCTAA | SEQ ID NO: 437 |
| | | | ACCAAGTTCCTTCACACTGGAGTATTTTG | |
| NM_003504 | CDC45L | SEQ ID NO: 438 | CAAGTTTCTGGACGCACTTATTTCCCTCCTG | SEQ ID NO: 439 |
| | | | TCCTAGGAATTTGATTCTTCCAGAATGAC | |
| NM_005671 | D8S2298E | SEQ ID NO: 440 | GAATGAGGTTGAAATGTATGCAGTAAGGTAC | SEQ ID NO: 441 |
| | | | TCAGTAATTAATTGGTATTTTTTCCCAGC | |
| NM_003136 | SRP54 | SEQ ID NO: 442 | GCAAAATTGAACCAACAAATGGCCAAAATGA | SEQ ID NO: 443 |
| | | | TGGATCCTAGGGTTCTTCATCACATGGGT | |
| NM_003642 | HAT1 | SEQ ID NO: 444 | AGACTAATTAGCCCATATAAGAAAAAGCAGA | SEQ ID NO: 445 |
| | | | GAGATCTTGCTAAGATGAGAAAATGTCTC | |
| AK024898 | | SEQ ID NO: 446 | TCCAAAATGATTTCTGAACTATCTGCAGTGA | SEQ ID NO: 447 |
| | | | AAATGTATCTGATGGATTGTAGAGCAAAG | |
| NM_015895 | GMNN | SEQ ID NO: 448 | GCCGAAGTTTACCTCCACTAGTTCTTTGTAG | SEQ ID NO: 449 |
| | | | CAGAGTACATAACTACATAATGCCAACTC | |
| NM_005258 | GCHFR | SEQ ID NO: 450 | CTTCAAAGAGAAGAGCCTTGGGAAACAACTT | SEQ ID NO: 451 |
| | | | TTATGAATACTACGTCGATGACCCTCCCC | |
| NM_013412 | RABL2A | SEQ ID NO: 452 | GCTATAGTTAGGAATACACAAGCGGTAAAAT | SEQ ID NO: 453 |
| | | | CGAGTCCTTACAGCCATACCACAAGGTAC | |
| NM_006698 | BLCAP | SEQ ID NO: 454 | TTATCGAACGTGGTCTGATTTTGAGGTTTAG | SEQ ID NO: 455 |
| | | | CAATAGCTAGCTATATATGGTAGGTGCCG | |
| NM_002893 | RBBP7 | SEQ ID NO: 456 | AAGTACGAGAAATGTTTCTGTTGAATGTAAT | SEQ ID NO: 457 |
| | | | GCTACATGAATGCTTGATTTATCAAGCGC | |
| NM_014254 | TMEM5 | SEQ ID NO: 458 | GGCAAGTTGGTCAATGCTGCATGATGAGAGG | SEQ ID NO: 459 |
| | | | CCATATTTATGTAATTTCTTAGGAACGAT | |
| NM_014445 | SERP1 | SEQ ID NO: 460 | TTAACTTGAACTCATTCCTGATGTTTGATAC | SEQ ID NO: 461 |
| | | | CCTGGTTGAAAACAATTCAGTAAAGCATC | |
| NM_000790 | DDC | SEQ ID NO: 462 | GTTGATTAAAACATTATAGATTTTCATGTTC | SEQ ID NO: 463 |
| | | | TTGCAGTCATCAGAAGTGGTAGGAAAGCC | |
| NM_018186 | FLJ10706 | SEQ ID NO: 464 | CCAAGAAAGGATGGATAAGCTAAAACGTTAC | SEQ ID NO: 465 |
| | | | ATACATACTCTAGGGTGAAACTTATCACT | |
| NM_002945 | RPA1 | SEQ ID NO: 466 | TCTGTCAATTTCATTATCATCAAGCAGGAAT | SEQ ID NO: 467 |
| | | | TATGTCGTAAGTCACTGACCCTAACTGCA | |
| NM_006479 | PIR51 | SEQ ID NO: 468 | GACCTCTAGCCATTTTATGATTATGTTCTCT | SEQ ID NO: 469 |
| | | | GTAAAACTCTTCAAGACTTCAATGAGAAG | |
| NM_007081 | RABL2B | SEQ ID NO: 470 | GCTATAGTTAGGAATACACAAGCCGTAAAAT | SEQ ID NO: 471 |
| | | | CGAGTCCTTACAGCCATACCACAAGGTAC | |
| NM_002451 | MTAP | SEQ ID NO: 472 | ACATCTTTATTCTGCTAAAGAAGAGGATCAT | SEQ ID NO: 473 |
| | | | TGATTTCTGTACAGTCAGAACAGTACTTG | |
| NM_000179 | MSH6 | SEQ ID NO: 474 | GAAAAGCAAGAGAATTTGAGAAGATGAATCA | SEQ ID NO: 475 |
| | | | GTCACTACGATTATTTCGGGAAGTTTGCC | |
| NM_018571 | ALS2CR2 | SEQ ID NO: 476 | CAGAGTACTATGACAAGGAAACATCAGAATT | SEQ ID NO: 477 |
| | | | ACTAATCTAGCTAGTGTCATTTATTCTGG | |
| NM_006527 | SLBP | SEQ ID NO: 478 | ATTTGGTATTTGTAAATAGCACTAGTTAGAC | SEQ ID NO: 479 |
| | | | TCTTTAGAATACTCCAAGAGTTAGGGCAG | |
| NM_001809 | CENPA | SEQ ID NO: 480 | GGGGATGAATAGAAAACCTGTAAGCTTTGAT | SEQ ID NO: 481 |
| | | | GTTCTGGTTACTTCTAGTAAATTCCTGTC | |
| NM_017998 | C9orf40 | SEQ ID NO: 482 | GTAGACTAATGGTCGTTTTTCCTGTTTAAAG | SEQ ID NO: 483 |
| | | | GAGATAACTAATTTGAGCTGAAGCAATGC | |
| XM_496352 | | SEQ ID NO: 484 | AACCAACCCATAATTGCATTTTACTTGTCGT | SEQ ID NO: 485 |
| | | | GGTTCGATCTGATTGTATTGTCGAAGGAC | |
| NM_018509 | PRO1855 | SEQ ID NO: 486 | TACATAATACACACAATGAGATGAAGACAAT | SEQ ID NO: 487 |
| | | | ATAGAAGTCCGCATAGTCATCATAATCCC | |
| NM_033411 | RWDD2 | SEQ ID NO: 488 | CCTCATTTCTGACCTACTGAATGCTCAGCTG | SEQ ID NO: 489 |
| | | | TTTGGCATTTCCTTTGAATGACCCAGCAT | |
| NM_004147 | DRG1 | SEQ ID NO: 490 | GGAATTTTGATGACCTATTGGAAAAGATCTG | SEQ ID NO: 491 |
| | | | GGACTATCTGAAACTAGTGAGAATTTACA | |
| NM_003707 | RUVBL1 | SEQ ID NO: 492 | ACAGCATTGAGAAAGAGCATGTCGAAGAGAT | SEQ ID NO: 493 |
| | | | CAGTGAACTTTTCTATGATGCCAAGTCCT | |
| NM_018248 | FLJ10858 | SEQ ID NO: 494 | GCCAGGAATAAAAATTATTCCTGGATGCTAA | SEQ ID NO: 495 |
| | | | TATCTGTAGATTCTCTGGCATTTAGTCTC | |
| NM_001638 | APOF | SEQ ID NO: 496 | AAATGTTTGGGTGTTGAAGTCACAGGGTTTG | SEQ ID NO: 497 |
| | | | GTTTCGAATCTAGGCTCCACTTGTTAGAG | |
| NM_015417 | C20orf28 | SEQ ID NO: 498 | AAGGTTTGAAGGTTACGGCTCAGGGCTGCCC | SEQ ID NO: 499 |
| | | | CATTAAAGTCAGTGTTGTGTTCTAAAAAA | |
| AL049442 | FLJ14431 | SEQ ID NO: 500 | AAATCTGCTATTCTGAGAAGCATTGAATGAA | SEQ ID NO: 501 |
| | | | TTCTTAACAAGAAGACTCATCTGTAGCTG | |
| NM_003362 | UNG | SEQ ID NO: 502 | GTGAGCTTTATCAGATAAGAGACCGAGAGAA | SEQ ID NO: 503 |
| | | | GTAAGCTGGGTCTTGTTATTCCTTGGGTG | |
| NM_004300 | ACP1 | SEQ ID NO: 504 | GAAGTGTTTTAATTTCTACACACCCATAGTG | SEQ ID NO: 505 |
| | | | CACACTTGTATATTGAAAAGATAGGGAAG | |
| NM_138959 | VANGL1 | SEQ ID NO: 506 | AACAAACAAACAAAGGTCAAGCTCTAAGAAA | SEQ ID NO: 507 |
| | | | ATTACTGCTCAAACTTCACTACCCTGGAA | |
| NM_005758 | hnRNPA3 | SEQ ID NO: 508 | CACTTCCTACAGACCTGAATTCAAATTTGGA | SEQ ID NO: 509 |
| | | | TAGTCTGAGTTATTAAATTCCCAAAGACA | |
| NM_002417 | MKI67 | SEQ ID NO: 510 | CTGGATTTGAGAACAAATACCCACCCATTTC | SEQ ID NO: 511 |
| | | | CACCATGGTATGGTAACTTCTCTGAGCTT | |
| NM_000107 | DDB2 | SEQ ID NO: 512 | CGATACCCAGATCCTAATTTCAAAAGTTGTA | SEQ ID NO: 513 |
| | | | CCCCTTATGAATTGAGGACGATCGACGTG | |
| Contig32050_RC | FLJ12973 | SEQ ID NO: 514 | CTACTCTCAATCAAATTAGTACCATCATTTA | SEQ ID NO: 515 |
| | | | AGCTTTGAATACTTGGCAGTAATTGCCTG | |
| NM_000291 | PGK1 | SEQ ID NO: 516 | ACTTAGCATTTTCTGCATCTCCACTTGGCAT | SEQ ID NO: 517 |
| | | | TAGCTAAAACCTTCCATGTCAAGATTCAG | |
| NM_012323 | MAFF | SEQ ID NO: 518 | AGTGGCGTATGGCCCTGAGCTGGGCTTTATA | SEQ ID NO: 519 |
| | | | TTATATATCTGCAAATAAATCACATTTTA | |
| NM_014752 | KIAA0102 | SEQ ID NO: 520 | GGATTTTCTGACACAGATATGAGAAGTTGTA | SEQ ID NO: 521 |
| | | | GCTCTGATGTCTAGCTGTAGTCTCCTTGA | |
| BC033874 | | SEQ ID NO: 522 | GCAATTCGAAGTCCCTTTAATTGTTTTGTGC | SEQ ID NO: 523 |
| | | | TTCCAACCTGTTTTGTTCCCGTTCAGATT | |
| NM_005733 | KIF20A | SEQ ID NO: 524 | TGCTTTACCATATATCAGGAATTATATCCAG | SEQ ID NO: 525 |
| | | | GATGCAATACTCAGACACTAGCTTTTTTC | |
| NM_004404 | NEDD5 | SEQ ID NO: 526 | CTGGAAAATCTGTAAAAAAGAAAAACAAGTT | SEQ ID NO: 527 |
| | | | TGCTAGTGACTAAGCCCCGCATATGTGAG | |
| NM_017664 | ANKRD10 | SEQ ID NO: 528 | ACTTGGTCTCTGGAGGTTTTACCACATGTAA | SEQ ID NO: 529 |
| | | | CAGATTCCTTTATATGTAGTGAAAATCAC | |
| NM_016448 | RAMP | SEQ ID NO: 530 | CATTTGGCTGTCAGAAATTATACCGAGTCTA | SEQ ID NO: 531 |
| | | | CTGGGTATAACATGTCTCACTTGGAAAGC | |
| NM_005659 | UFD1L | SEQ ID NO: 532 | CTTTGCCTGTCTGACCACCGGGGATGTGATT | SEQ ID NO: 533 |
| | | | GCCATTAACTACAATGAAAAGATCTACGA | |
| NM_001001551 | C9orf103 | SEQ ID NO: 534 | ACCCCAATGTGTCAAGACAGACTTGTTTAGG | SEQ ID NO: 535 |
| | | | TGTAATTTTAGGAATTATGCTGGTTCATC | |
| NM_001790 | CDC25C | SEQ ID NO: 536 | ATAAGGCAGCCTTGAGTTGCATAGAGATTTG | SEQ ID NO: 537 |
| | | | TATTGGTTCAGGGAACTCTGGCATTCCTT | |
| NM_000481 | AMT | SEQ ID NO: 538 | AACCTACCTCACCATGGTTTTTCACATTGCA | SEQ ID NO: 539 |
| | | | AAGGGTAATAACATGGGCAGTGCGGACTT | |
| AL080182 | NDST2 | SEQ ID NO: 540 | GTCTTTTTGTAACTTATTTAACCCCAACATA | SEQ ID NO: 541 |
| | | | TCTTGGTATCCTCCATTAACACCACAGGG | |
| NM_016567 | BCCIP | SEQ ID NO: 542 | GGGAACCTTCATGACTGTTGGAATTGCTCTG | SEQ ID NO: 543 |
| | | | TCATAATAAGTCAGGGATATTTAGGGGGC | |
| NM_012396 | PHLDA3 | SEQ ID NO: 544 | TGGCCATTAGCATTTCATGTCTTTCCCCAAA | SEQ ID NO: 545 |
| | | | TGAAATGCCCTGCAAAGGGCAGTAACCCC | |
| NM_016488 | PPHLN1 | SEQ ID NO: 546 | GCCTTGGTTATCAACAGGTGGAATGTAGATA | SEQ ID NO: 547 |
| | | | TTGTAAAGCTTTTTGTGAATTAAAAGTGC | |
| NM_001262 | CDKN2C | SEQ ID NO: 548 | CCCTTGCTTCCCCTTTTGCCAATCTCAACAC | SEQ ID NO: 549 |
| | | | CCAAGTTGAAGACTTTGTTTTTAAAATGG | |
| U81002 | C15orf23 | SEQ ID NO: 550 | AGTCCGATTCCTAGAACAGCAAACCTTATGT | SEQ ID NO: 551 |
| | | | AACAATCAAGTAAATGATTTAACAACAGC | |
| NM_005132 | REC8L1 | SEQ ID NO: 552 | TCTGAATGTGCATTTCCAGCCTTCTTGCTCT | SEQ ID NO: 553 |
| | | | CAGAGCTATTGTTCAAGCAGAAAACAAGC | |
| NM_012429 | SEC14L2 | SEQ ID NO: 554 | CCTGGGTTTACAACGCTGTTAGGAAAATTAA | SEQ ID NO: 555 |
| | | | CCAATGAATAAAGCAACGTTCAGTGCGCA | |
| NM_017815 | C14orf94 | SEQ ID NO: 556 | TTTTGTCCGACACTTACACTGTTGAGAAAGT | SEQ ID NO: 557 |
| | | | GGAAGTTCATCGTCTGATTAGGGACCGTT | |
| NM_001823 | CKB | SEQ ID NO: 558 | TCACCCAGATTGAAACTCTCTTCAAGTCTAA | SEQ ID NO: 559 |
| | | | GGACTATGAGTTCATGTGGAACCCTCACC | |
| NM_003160 | AURKC | SEQ ID NO: 560 | AAATGATTGAGGGGAGAACATATGATGAAAA | SEQ ID NO: 561 |
| | | | GGTGGATTTGTGGTGCATTGGAGTGCTCT | |
| NM_001321 | CSRP2 | SEQ ID NO: 562 | CAACTCTGACTGAAAAAGAAGGTGAAATCTA | SEQ ID NO: 563 |
| | | | TTGTAAAGGATGCTATGCAAAGAACTTTG | |
| NM_018518 | MCM10 | SEQ ID NO: 564 | ATAGAGTGTTCACTTCTTTATCATAACAAAA | SEQ ID NO: 565 |
| | | | TTCTAGTGTTTATACGAACACCCAGAGGC | |
| NM_000363 | TNNI3 | SEQ ID NO: 566 | TGTGGACAAGGTGGATGAAGAGAGATACGAC | SEQ ID NO: 567 |
| | | | ATAGAGGCAAAAGTCACCAAGAACATCAC | |
| NM_016359 | NUSAP1 | SEQ ID NO: 568 | ACATTGCTTACTTAAAAGCTACATAGCCCTA | SEQ ID NO: 569 |
| | | | TCGAAATGCGAGGATTAATGCTTTAATGC | |
| NM_018244 | C20orf44 | SEQ ID NO: 570 | CTTGCTATAAAATGAGTCATATAAAGAAACT | SEQ ID NO: 571 |
| | | | CTATATGGGTGAGGTATATCCCACTTCTG | |
| NM_003276 | TMPO | SEQ ID NO: 572 | AAAGTAATTGCCTGTGTAGAACTACTTGTCT | SEQ ID NO: 573 |
| | | | TTTCTAAAGATTTGCGTAGATAGGAAGCC | |
| NM_016556 | HUMGT198A | SEQ ID NO: 574 | GCTACAGAGCTGTCTGATGCAATACTTGAAG | SEQ ID NO: 575 |
| | | | GATACCCCAAGAGCAAGAAGCAGTTCTTT | |
| NM_016095 | Pfs2 | SEQ ID NO: 576 | ATGGATTCAGGATGTTGTTGGAGAAACAAGT | SEQ ID NO: 577 |
| | | | TTGTGATTAGTCCTTAAAACTTAGCTCCC | |
| NM_014670 | BZW1 | SEQ ID NO: 578 | ACTCAGTAATGACTCAAGCCTCTGGCTATTA | SEQ ID NO: 579 |
| | | | ACATACCCTAGTTGCCGTTTTTTAATTGC | |
| NM_003483 | HMGA2 | SEQ ID NO: 580 | GTTGTACAATCAAAACACACTACTACCTCTT | SEQ ID NO: 581 |
| | | | AAGTCCCAGTATACCTCATTTTTCATACT | |
| NM_018131 | C10orf3 | SEQ ID NO: 582 | TTTATCCAAGCACTTAGAAAACCTACAATCC | SEQ ID NO: 583 |
| | | | TAATTTTGATGTCCATTGTTAAGAGGTGG | |
| NM_013290 | HUMGT198A | SEQ ID NO: 584 | AAATTGCAGTAGCTTGAGGTTAACATTTAGA | SEQ ID NO: 585 |
| | | | CTTGGAACAATGCTAAAGGAAAGCATTTG | |
| NM_020347 | LZTFL1 | SEQ ID NO: 586 | TGGATTTCTAAGGATGGTGGTTTGAGCCTTG | SEQ ID NO: 587 |
| | | | ATTAGACTTTTGATGTGCTAAGCCAGACA | |
| NM_012177 | FBXO5 | SEQ ID NO: 588 | GATTATTGTACGAAGTGTCTCTGTAATTATC | SEQ ID NO: 589 |
| | | | ATACTACTAAAGACTGTTCAGATGGCAAG | |
| NM_182705 | MGC45871 | SEQ ID NO: 590 | GTGTGAAATACGTCAGTGAAGTAAGTAGCAG | SEQ ID NO: 591 |
| | | | TGAGCGATTGTGAATGTGTAATGTAAATG | |
| NM_018204 | CKAP2 | SEQ ID NO: 592 | TCTCTCCACACATAAATAACACCACTAAAGT | SEQ ID NO: 593 |
| | | | TGTTTTGTAAGGTTCCAAACTAATATGGC | |
| CR622106 | | SEQ ID NO: 594 | TGGAAATCACACTTGTGAGGTATGGTATCCA | SEQ ID NO: 595 |
| | | | TTATCAACAGTGGATTCATGATTGACTTC | |
| AK001936 | | SEQ ID NO: 596 | GGAGTCCATCATTTAGCCAGTATACATAGAG | SEQ ID NO: 597 |
| | | | GAACTGCTTCGAATCAAGGCAACTGGTGA | |
| NM_012286 | MORF4L2 | SEQ ID NO: 598 | AAGCTCCTTTTCTCATTTCAAAGTTGCTACC | SEQ ID NO: 599 |
| | | | AGTGTATGCAGTAATTAGAACAAAGAAGA | |
| NM_017613 | DONSON | SEQ ID NO: 600 | TGAGGAAAAAAGCCTTCTAGCAAGGAAATTC | SEQ ID NO: 601 |
| | | | AAGATTCTTGAAGTTGAAGGAATATACTG | |
| NM_014584 | ERO1L | SEQ ID NO: 602 | AGGTCCTACCTATATTCCAAATACTTTCACT | SEQ ID NO: 603 |
| | | | CCCTTCACTTTACAGCAAGGGTCAGTAGA | |
| AA598803_RC | LOC139886 | SEQ ID NO: 604 | GTATTTTATGTCACCTAGATCTAATTCTGAA | SEQ ID NO: 605 |
| | | | AACATTGTAATAAAATAATTAGCTATAAT | |
| NM_013352 | SART2 | SEQ ID NO: 606 | TTAATGGTATACTCATGTTGAAAGATAAATG | SEQ ID NO: 607 |
| | | | TTGCTAAGTCCTGGTATGATGGTGTGAGC | |
| BC043009 | LOC338620 | SEQ ID NO: 608 | CTTGAGCACCTCATCTTTTGCTCAAATTGAA | SEQ ID NO: 609 |
| | | | ATGTCATCGAACTGTATTTCTCAAGTCAA | |
| NM_002875 | RAD51 | SEQ ID NO: 610 | CTATTGGAGGAAATATCATCGCCCATGCATC | SEQ ID NO: 611 |
| | | | AACAACCAGATTGTATCTGAGGAAAGGAA | |
| NM_014685 | HERPUD1 | SEQ ID NO: 612 | GAAGAGCTTTAATATATACTCTATGTAGTTT | SEQ ID NO: 613 |
| | | | AATAAGCACTGTACGTAGAAGGCCTTAGG | |
| NM_001034 | RRM2 | SEQ ID NO: 614 | GGTAGTATTGTAAAATTTCAAGTCATCCTTA | SEQ ID NO: 615 |
| | | | AACAAAATGATCCACCTAAGATCTTGCCC | |
| NM_001613 | ACTA2 | SEQ ID NO: 616 | CCTAGCCAAAGCTCTGACTCGTTACCTATGT | SEQ ID NO: 617 |
| | | | GTTTTTTAATAAATCTGAAATAGGCTACT | |
| NM_001343 | DAB2 | SEQ ID NO: 618 | TCTCCTTAGACACTTTGGAATCTAACCACTT | SEQ ID NO: 619 |
| | | | AAGGACCTTTTTAAAGAGATAGCTTCTCT | |
| Contig23667_RC | | SEQ ID NO: 620 | AACCTACAGAAAATTTGAAATTTGACAGTAA | SEQ ID NO: 621 |
| | | | GCACCTGTATATCCTTCCTCTAGTTTGTC | |
| BU743075 | | SEQ ID NO: 622 | TGTTCCAAGTAGGTTAGAACCATGGAAAAGA | SEQ ID NO: 623 |
| | | | GATTGCAAATGGTAGTTTCTTCTAGATAT | |
| NM_006143 | GPR19 | SEQ ID NO: 624 | AGTTCTTCAGCCTCTAAACCTACTCTGTATT | SEQ ID NO: 625 |
| | | | CAATTTATAATGCCAATTTTCGGAGAGGG | |
| NM_006145 | DNAJB1 | SEQ ID NO: 626 | TTCATGGACTATGGACTCTTTCAAAGGGATC | SEQ ID NO: 627 |
| | | | TGATCCTTTTGAATTTTGCACAGCCCTAG | |
| NM_019018 | FLJ11127 | SEQ ID NO: 628 | ACAAGTAGGGAAAAACAGCTAACCTGGAGAG | SEQ ID NO: 629 |
| | | | AAAGAATTTCTTTAACCTTTATGTTCTTC | |
| NM_002816 | PSMD12 | SEQ ID NO: 630 | AAATGTGGAGAAGATAACACTTGATTCCATT | SEQ ID NO: 631 |
| | | | TCATTGTCATTAGTGTATTAACCAGCAGG | |
| NM_004900 | APOBEC3B | SEQ ID NO: 632 | TGCTCACAGACACCAGCAAAGCAATGTGCTC | SEQ ID NO: 633 |
| | | | CTGATCAAGTAGATTTTTTAAAAATCAGA | |
| AX746755 | | SEQ ID NO: 634 | GTGGAGTAGGTTGAGGTGTAGCCATGACATT | SEQ ID NO: 635 |
| | | | CTGGAAACTACATTTTTTGTAATAGTCAT | |
| NM_203394 | E2F7 | SEQ ID NO: 636 | ATTCTGGTACAAGAGTCGGGGGTATAACTTT | SEQ ID NO: 637 |
| | | | TATACTTGAATCTACCTACCAAGTTTACA | |
| NM_004414 | DSCR1 | SEQ ID NO: 638 | AATGCCAAGTAACAATGCATGCTTTGGAAAT | SEQ ID NO: 639 |
| | | | TTGGAAGATGGTTTTATTCTTTGAGAAGC | |
| NM_003447 | ZNF165 | SEQ ID NO: 640 | CCCAAAACTTGCTAAACATGCAGCAGTTTTC | SEQ ID NO: 641 |
| | | | AGTGGAGATAAAACTCATCAGTGTAATGA | |
| NM_002265 | KPNB1 | SEQ ID NO: 642 | GAAGTGTGTTAGTCCAACTTTAAGAAAAATC | SEQ ID NO: 643 |
| | | | CAAACTCATCAGCTTTTGATAGCATCTTG | |
| NM_006163 | NFE2 | SEQ ID NO: 644 | GATGACAATGGCAACAAGTGTTTGGAAGTTC | SEQ ID NO: 645 |
| | | | CAAGGTGTGTTCAAAGAGGCTTGCCTTGA | |
| AK024294 | | SEQ ID NO: 646 | CTGTGACCTACAAACCCTTACTTAATTCAGT | SEQ ID NO: 647 |
| | | | GTTACTATAAATGATTCTTCCCTTAAACC | |
| NM_017957 | EPN3 | SEQ ID NO: 648 | GCAGATCAGTTTATTTAATGCTTGCAACGAC | SEQ ID NO: 649 |
| | | | TCTCTGAGGTAGAAAATATTGTTAATTCC | |
| NM_004111 | FEN1 | SEQ ID NO: 650 | AGCGTGACCCTTTTCAGTAGTGCTAGTCCCT | SEQ ID NO: 651 |
| | | | TTTTTACTTGATCTTAATGGCAAGAAGGC | |
| NM_012347 | FBXO9 | SEQ ID NO: 36 | GGTTCAACAAACTCATCTGGATACATCATTC | SEQ ID NO: 652 |
| NM_033480 | | SEQ ID NO: 37 | TTGTCACATTACTTACAAATCAACTGGTG | |
| NM_033481 | | SEQ ID NO: 38 | | |
| NM_001969 | EIF5 | SEQ ID NO: 653 | ACATCATGCTACACTTTACACTAAAAATCTA | SEQ ID NO: 654 |
| | | | TTACTGTGAGTGTGAAAAACTAGTGGTGG | |
| NM_001254 | CDC6 | SEQ ID NO: 655 | CAAGGAAACCCGTTTGACAAAGGTGTTTTTC | SEQ ID NO: 656 |
| | | | AAGATTGAAGAGAAAGAAATAGAACATGC | |
| NM_003258 | TK1 | SEQ ID NO: 657 | CTTCCTACCTCTGGTGATGGTTTCCACAGGA | SEQ ID NO: 658 |
| | | | ACAACAGCATCTTTCACCAAGATGGGTGG | |
| NM_004091 | E2F2 | SEQ ID NO: 659 | TATGAGCACCATGTAAGCCTCCTTGTATTGA | SEQ ID NO: 660 |
| | | | GATAATTGGGCATTAAACATTAAACTGCA | |
| NM_018154 | ASF1B | SEQ ID NO: 661 | TCCAGCTTTCTGGTGTCACAGGCGGGAATGT | SEQ ID NO: 662 |
| | | | TAGTTAGTAGGTAGACTTAGATCCCATTT | |
| NM_000040 | APOC3 | SEQ ID NO: 663 | GAAAGACTACTGGAGCACCGTTAAGGACAAG | SEQ ID NO: 664 |
| | | | TTCTCTGAGTTCTGGGATTTGGACCCTGA | |
| AK002107 | | SEQ ID NO: 665 | ACACAGTAGCAAAAGAGAAGATCTCATTTAC | SEQ ID NO: 666 |
| | | | AAATATCTATGGTGTTTCCTTGTTCTGTG | |
| NM_021175 | HAMP | SEQ ID NO: 55 | TATTTATTCCTGCTGCCCCAGAACATAGGTC | SEQ ID NO: 56 |
| | | | TTGGAATAAAATGGCTGGTTCTTTTGTTT | |
| NM_003282 | TNNI2 | SEQ ID NO: 140 | CTCTGCAAACAGCTGCACGCCAAGATCGATG | SEQ ID NO: 141 |
| | | | CGGCTGAAGAGGAGAAGTACGACATGGAG | |
| AB033092 | MTA3 | SEQ ID NO: 667 | GTCTGTAAGGAGATGCCATCTACTAACCAAT | SEQ ID NO: 668 |
| | | | TTGTATTGTGTTTCCAATAAATTCCTGGA | |
| NM_005498 | AP1M2 | SEQ ID NO: 669 | TTATATGAAGAAATAGAAGAGGGGCTTGAAG | SEQ ID NO: 670 |
| | | | TCCCCCTCGCGAGTGCCTTCTTGCAATTA | |
| Contig43632_RC | | SEQ ID NO: 671 | TCTGATCATTTATACTAGCTACTCAGTCTCT | SEQ ID NO: 672 |
| | | | ACTTTTTCTAGAAGTCCCCAGTAACCAAA | |
| NM_002309 | LIF | SEQ ID NO: 673 | TCCTTTCCACTGAAAAGCACATGGCCTTGGG | SEQ ID NO: 674 |
| | | | TGACAAATTCCTCTTTGATGAATGTACCC | |
| NM_000167 | GK | SEQ ID NO: 675 | AGGAAGAATTCGGATCCTTACCATTGGAATC | SEQ ID NO: 676 |
| | | | TTCCATCGAACATACTCAAACACTTTTGG | |
| NM_001082 | CYP4F2 | SEQ ID NO: 677 | TTTTTTCTGTCCAGGTTGTTCATATAATAAT | SEQ ID NO: 678 |
| | | | ATGCTGTGAGCATCTTTCCATGACATTAA | |
| NM_001657 | AREG | SEQ ID NO: 679 | GTTGCTGTTATTACAGTCCAGCTTAGAAGAC | SEQ ID NO: 680 |
| | | | AATACGTCAGGAAATATGAAGGAGAAGCT | |
| NM_003670 | BHLHB2 | SEQ ID NO: 681 | GACTCAGTTTTCAATTCCATCCTAAAACTCC | SEQ ID NO: 682 |
| | | | TTTTAACCAAGCTTAGCTTCTCAAAGGCC | |
| NM_000799 | EPO | SEQ ID NO: 683 | TCATGGGGTCCAAGTTTTGTGTATTCTTCAA | SEQ ID NO: 684 |
| | | | CCTCATTGACAAGAACTGAAACCACCAAA | |
| NM_015922 | NSDHL | SEQ ID NO: 685 | ACGATGAGGGCGGCAAAAACAGACATTTCTT | SEQ ID NO: 686 |
| | | | CCTTCATGGAACTGGATTTGGATTTCTTG | |
| AK123483 | | SEQ ID NO: 687 | CAGCCTTTCCTCATGTCAACACAGTTCACAA | SEQ ID NO: 688 |
| | | | TATAGTTTTCAAAGTACAGTTTAAAACTC | |
| NM_016371 | HSD17B7 | SEQ ID NO: 689 | ACACTCTGGAACATTGCATACCTTCTGTACA | SEQ ID NO: 690 |
| | | | TTCTGGGGTACATGGATTTCTACTGAGTT | |
| NM_139164 | STARD4 | SEQ ID NO: 691 | AGGGCTGAGTAATTGCCTTTCATACAAATAA | SEQ ID NO: 692 |
| | | | AAGCTTTAACTCTTTTCTCAAGAGTGTAC | |
| NM_002130 | HMGCS1 | SEQ ID NO: 693 | TAAAATAACAGCAAGTTTATGTGATCTTAAA | SEQ ID NO: 694 |
| | | | TCAAGGCTTGATTCAAGAACTGGTGTGGC | |
| NM_005261 | GEM | SEQ ID NO: 695 | ACTAGGCTCATCAGAATCGTGACTATTATTG | SEQ ID NO: 696 |
| | | | CTCCATCAAACTGTGAAAAGAAATGATGT | |
| NM_000859 | HMGCR | SEQ ID NO: 697 | GCTCTTGGAAAGCTGATGATGTCTCTTGTAG | SEQ ID NO: 698 |
| | | | AGATGACTCTGAAAAACATTCCAGGAAAC | |
| NM_005252 | FOS | SEQ ID NO: 699 | ATGGAGTGTGTATTGTTCCCAGTGACACTTC | SEQ ID NO: 700 |
| | | | AGAGAGCTGGTAGTTAGTAGCATGTTGAG | |
| NM_003129 | SQLE | SEQ ID NO: 700 | GTTGCAATCTATGCCGTGTATTTTTGCTTTA | SEQ ID NO: 702 |
| | | | AGTCAGAACCTTGGATTACAAAACCTCGA | |
| NM_000431 | MVK | SEQ ID NO: 703 | GGCCTTGGTTTATATGCACTTTCTTCCGATC | SEQ ID NO: 704 |
| | | | TGTACCTGAGAGGTTTGTGGAAAAGATGG | |
| NM_004508 | IDI1 | SEQ ID NO: 705 | CTTGTAAATAGTATTTACCAGTTAGCAAAGT | SEQ ID NO: 706 |
| | | | CTGTGTTTTCAGAATTACAGTGAGCACAG | |
| NM_006745 | SC4MOL | SEQ ID NO: 707 | GAATTCACCTAAAGATCAAAATATCATGGAT | SEQ ID NO: 708 |
| | | | TGAACCTCATCAATTGATAGCAGTGAGTG | |
| XM_373497 | LOC387763 | SEQ ID NO: 709 | ACCTGGATATGTCTGTGAGGCTCCTGAAAGG | SEQ ID NO: 710 |
| | | | AGACAAATAAAGTCAATATATTTGCACAA | |
| AK001732 | SMU1 | SEQ ID NO: 711 | AAGCTGGTAGTTATGATGTGCATAGTTTGTA | SEQ ID NO: 712 |
| | | | TCACCGTTTATGAAGATGGAGAATCTAGT | |
| NM_017785 | FLJ20364 | SEQ ID NO: 713 | GCCTCAAAAGTTACTATGGTGCTTAAGATTG | SEQ ID NO: 714 |
| | | | TCTTGATCTGACATATATCACCTTCTGGG | |
| NM_006895 | HNMT | SEQ ID NO: 715 | TGAGATGTAGCAAATTCCAATACATTATTGG | SEQ ID NO: 716 |
| | | | ACTTCCATTTGGAATCATATGGGATACTG | |
| NM_001813 | CENPE | SEQ ID NO: 717 | TGTAGTGCAGCTTAATTTCAATTCAGTCTTT | SEQ ID NO: 718 |
| | | | ACTTTGCCACTAGAGTTGAAAGATAAGGG | |
| NM_006644 | HSPH1 | SEQ ID NO: 719 | GTGGAATGGAAGAAATGTCGATCCTGTTGTA | SEQ ID NO: 720 |
| | | | ACTGATTGTGAACACTTTTATGAGCTTTA | |
| NM_005813 | PRKCN | SEQ ID NO: 721 | CTGAGTCACTAGGCAATCAATCTACTGTGGT | SEQ ID NO: 722 |
| | | | TTTACTATGTAAGGTGAAAATTAACTGGA | |
| NM_018229 | C14orf108 | SEQ ID NO: 723 | CTTTAAGTGACCATTCAAGAAAAGATGAAAT | SEQ ID NO: 724 |
| | | | CTCACGAACCTCAAAACTTCATTCATGTC | |
| NM_000354 | SERPINA7 | SEQ ID NO: 725 | GGCCATTGGCTAATTGCACGTGTGTATTGCA | SEQ ID NO: 726 |
| | | | ATGGGAAATAAATAAATAATATAGCCTGG | |
| AK022936 | | SEQ ID NO: 727 | GGAGTGAAGGTCTTATCTGCATTAATTTTGT | SEQ ID NO: 728 |
| | | | TAATGCACATTGTAGATGTGGAGAACATC | |
| BG897660 | | SEQ ID NO: 729 | TGTAAGAAAAGAGACATGGCCATTTCTAAAC | SEQ ID NO: 730 |
| | | | TATATAAGGTGAGTGTGTCTATTCCCAGC | |
| AB040937 | MGC16943 | SEQ ID NO: 731 | TAGTTGTTCTTGATCATGTGACTTGTAATCA | SEQ ID NO: 732 |
| | | | GGATTTAGAACGACAAAAGCCTTTACTAC | |
| NM_018685 | ANLN | SEQ ID NO: 733 | TTCCATGCTATCTAGAGGTTTTTGATGTCAT | SEQ ID NO: 734 |
| | | | CTTAAGAAACACACTTAAGAGCATCAGAT | |
| AK054652 | | SEQ ID NO: 735 | GAAGAAATGTTCCAGGATACACTTGGATTAG | SEQ ID NO: 736 |
| | | | TTATGCAGTATTTCTTATCAATGTCTAGC | |
| NM_012415 | RAD54B | SEQ ID NO: 737 | GAGAAGAAGTTCATACAGGTGATTCGTTGGA | SEQ ID NO: 738 |
| | | | AAAATTCATTGTCTCTAGAGATTGTCAGC | |
| NM_021141 | XRCC5 | SEQ ID NO: 739 | CTACCTAAGTCAGCTTTCATCTTTGTGGATG | SEQ ID NO: 740 |
| | | | GTGTCTCCTTTACTAAATAAGAAAATAAC | |
| NM_000672 | ADH6 | SEQ ID NO: 741 | GGTTGCTGATTATATGGCAGAGAAGTTGAAT | SEQ ID NO: 742 |
| | | | CTAGATCCACTAATTACTCATACTCTGAA | |
| NM_003035 | SIL | SEQ ID NO: 743 | TACAAGAGTGTATTTCCCCTTAATTAGGATG | SEQ ID NO: 744 |
| | | | CATGTTGATTAAACTCGAGATACAGCTTT | |
| NM_024624 | SMC6L1 | SEQ ID NO: 745 | GTTGTAGTTTTGAATGTTGTGCTCTTTCATA | SEQ ID NO: 746 |
| | | | CAGAACGGGAAACATAATCCTCAGGTATC | |
| NM_012115 | CASP8AP2 | SEQ ID NO: 747 | AAATGAGCACTAGAGGGAGGTTTATCACCTT | SEQ ID NO: 748 |
| | | | TTTGGTTACCAGATGATAATCTATATTTG | |
| NM_001067 | TOP2A | SEQ ID NO: 749 | GATGATTCTGACTCTAATTTTGAGAAAATTG | SEQ ID NO: 750 |
| | | | TTTCGAAAGCAGTCACAAGCAAGAAATCC | |
| AK093898 | | SEQ ID NO: 751 | GCACTTACTAGGCTAAACTTTCTGAAACTGA | SEQ ID NO: 752 |
| | | | TTGGTTTTGACCTATGAGATAAGGAAACA | |
| NM_017669 | FLJ20105 | SEQ ID NO: 753 | AGTGAAACTGAATATGAGGGAATTTTTGTTC | SEQ ID NO: 754 |
| | | | CCATAATTGGATTCTTTGGGAACATGAAG | |
| NM_001033 | RRM1 | SEQ ID NO: 755 | TAACTCATGAGAAGTACTGATAGGACCTTTA | SEQ ID NO: 756 |
| | | | TCTGGATATGGTCCTATAGGTTATTCTGA | |
| NM_004673 | ANGPTL1 | SEQ ID NO: 178 | TGTGAAGATAATCATTTGGACAACTCAAATC | SEQ ID NO: 757 |
| | | | CATCAACATGACCAATGTTTTTCATCTGC | |
| NM_133496 | SLC30A7 | SEQ ID NO: 758 | CAGTTAGCCTCAATGAAGATTCTATTTTGAA | SEQ ID NO: 759 |
| | | | CTAGACATCCTGTCAATAGTTTGTCAAGT | |
| NM_014829 | DDX46 | SEQ ID NO: 760 | GTGATCATTTCCTAAGAAAAGGTAAAATTCA | SEQ ID NO: 761 |
| | | | CTATCATATTTTGTGTCCCCACCTTGATG | |
| NM_000185 | SERPIND1 | SEQ ID NO: 762 | CATGCTGTAAGCTCATAGAAGTCACTGTAAC | SEQ ID NO: 763 |
| | | | TGTAGTGTGTCTGCTGTTACCTAGAGGGT | |
| NM_001316 | CSE1L | SEQ ID NO: 764 | AAGGTATCTGGAAATGTAGAGAAAAAGATCT | SEQ ID NO: 765 |
| | | | GTGCGGTTGGCATAACCAACTTACTAACA | |
| NM_004766 | COPB2 | SEQ ID NO: 766 | CTGGATGAAGATATTTTGGATGATTGACTGT | SEQ ID NO: 767 |
| | | | AATGCTTTCCATTTACCTGACTAAACAGA | |
| NM_006855 | KDELR3 | SEQ ID NO: 768 | GCCTTGTACTTAGTACCTTAATACCAATAAC | SEQ ID NO: 769 |
| | | | CTAATGGTACTTAGGCGAGTACCATTTGC | |
| Contig35294_RC | BRIP1 | SEQ ID NO: 770 | AAGAATTTAGAATATATTAGATCCCATCTAG | SEQ ID NO: 771 |
| | | | TATTATATATTTTTTCTAGTTGATCATTG | |
| NM_018206 | VPS35 | SEQ ID NO: 772 | GGTTTTAAACCAGCTTATCCAAAAGATTCGA | SEQ ID NO: 773 |
| | | | GAAGACCTCCCGAATCTTGAATCCAGTGA | |
| NM_005915 | MCM6 | SEQ ID NO: 774 | TTGGTAGTTAACCCTAACTACTTGCTCGAAG | SEQ ID NO: 775 |
| | | | ATTGAGATAGTGAAAGTAACTGACCAGAG | |
| NM_030917 | FIP1L1 | SEQ ID NO: 776 | TGTAATGCTGAAATTCAACTCTAACCAGTTC | SEQ ID NO: 777 |
| | | | TGAATCTATCGTCATACATACACATCATC | |
| NM_006704 | SGT1 | SEQ ID NO: 778 | TAAAACCTGTAGTGAGTTCTTCAGTCAGTCA | SEQ ID NO: 779 |
| | | | ACACTAAACCTGTTTTGTAAATTTGATGC | |
| NM_001539 | DNAJA1 | SEQ ID NO: 780 | TTTATCGTAGACCATATGAAAAGGGTCGCCT | SEQ ID NO: 781 |
| | | | AATCATCGAATTTAAGGTAAACTTTCCTG | |
| AK095780 | | SEQ ID NO: 782 | CTGGCTCATAGCATTTCACAAACATTATACT | SEQ ID NO: 783 |
| | | | TCAGAGTCCCAAAGCCTTTAAATAAAATG | |
| NM_021163 | RBAK | SEQ ID NO: 787 | GGCAACTACTAAGATTTCCTACGCTGTCCTT | SEQ ID NO: 785 |
| | | | AAAGCTTAGCATAATGTATATTTTAAGTG | |
| NM_006267 | RANBP2 | SEQ ID NO: 786 | AATCTTAACTATTGGTGTACTCATTTATGCA | SEQ ID NO: 787 |
| | | | TAGAGACTCGTTTATGAATGGGTAGAGCC | |
| NM_002614 | PDZK1 | SEQ ID NO: 788 | GTCTTACAGGCGGCTATTGCAGACGGCTAAT | SEQ ID NO: 789 |
| | | | TTATGCTTAACTTAGGAAGAGATAAGGCA | |
| NM_001326 | CSTF3 | SEQ ID NO: 790 | GTGGTATGTTTAAAAGAGACAACGCTTTGTT | SEQ ID NO: 791 |
| | | | ACAAGGTTCTTGGAAACAAAGTTGTATTG | |
| NM_000465 | BARD1 | SEQ ID NO: 792 | AGCATGTCTACGAAGAAAAGTATGTGAACAG | SEQ ID NO: 793 |
| | | | GAAGAAAAGTATGAAATTCCTGAAGGTCC | |
| NM_003115 | UAP1 | SEQ ID NO: 794 | GCCAACCATTTCTTCACTGTACCATTTCTGA | SEQ ID NO: 795 |
| | | | GAGATGTTGTCAATGTTTATGAACCTCAG | |
| BU616254 | | SEQ ID NO: 796 | AGTTCAAAATGAGTGAGAGAGAACTTTATGC | SEQ ID NO: 797 |
| | | | AGGTTGAGATAATGCCTAAAATAATGAGC | |
| Contig35088_RC | | SEQ ID NO: 798 | TAGTTAGCCGTACTTTAGTACTAGAAACAGT | SEQ ID NO: 799 |
| | | | AAATTGACATCCTTGAACTAATTCCTACC | |
| U83115 | AIM1 | SEQ ID NO: 800 | TCTAATTCTAAAATGCTGATCTTCTCTGGAG | SEQ ID NO: 801 |
| | | | TCTATGGTAGGCAATTATGGTCACTGGAA | |
| NM_016618 | LOC51315 | SEQ ID NO: 802 | TGTGAATAGGTTCTTTAACTTCTAACAAAGG | SEQ ID NO: 803 |
| | | | CCTAGTAAACAAAGTGTTTAGCATGCTTG | |
| NM_006267 | RANBP2 | SEQ ID NO: 804 | CAAAATGACATGTAAACTGACTTTTCCCGTA | SEQ ID NO: 805 |
| | | | TTAGTATTCCAAAGATGCTTAAAAGTGGC | |
| NM_003368 | USP1 | SEQ ID NO: 806 | ATCTGTTTAACTCATATTCTGCACGATCTGT | SEQ ID NO: 807 |
| | | | ATATAGTACATCAAACTTAGAGGTGTGAC | |
| NM_012484 | HMMR | SEQ ID NO: 808 | TCACTTGGTCCTACCTATTATCCTTCTACTT | SEQ ID NO: 809 |
| | | | GTCCAGTTCAAATAAGAAATAAGGACAAG | |
| NM_000057 | BLM | SEQ ID NO: 810 | TAAGCCTTCATATGCATTCTCATAACAACCG | SEQ ID NO: 811 |
| | | | AATCTCAATGTACATAGACCCTCTTTCTT | |
| AB002330 | SR140 | SEQ ID NO: 812 | GGAACATGTGACTGTAAAATCTCACATTTAC | SEQ ID NO: 813 |
| | | | AAAGTGCTTGATCTCTTCATATTTCACAC | |
| AL117611 | | SEQ ID NO: 814 | TGATTCACAGTAAAACTTTTCATGAATCATT | SEQ ID NO: 815 |
| | | | GAGGGTTAACTGTTATCACTTGCCAATGG | |
| NM_015515 | KRT23 | SEQ ID NO: 816 | ACAGTCTCACCAGACTTCTCATAATGCTCTT | SEQ ID NO: 817 |
| | | | AATATATTGCACTTTTCTAATCAAAGTGC | |
| NM_000562 | C8A | SEQ ID NO: 818 | TGGATGTTGACTGTTAACTAGAAGCTCTGTC | SEQ ID NO: 819 |
| | | | CTACTTACAGCACTTTGGATCATCAAAAA | |
| NM_016041 | F-LANa | SEQ ID NO: 820 | TATTGTCTGTCTCTGAATCCCTATAAAGCTT | SEQ ID NO: 821 |
| | | | CAAGTCTGTATGACATTCTTAACGCCAAA | |
| NM_015599 | PGM3 | SEQ ID NO: 822 | AATGAATATCCTTGTACCCTCTATATAGCTA | SEQ ID NO: 823 |
| | | | ACTTCCAAATGTAAGAGAACTATGGGGTA | |
| NM_006895 | HNMT | SEQ ID NO: 824 | GGCCTTTTTCTCGTCCTTATTAAACCCTTAG | SEQ ID NO: 825 |
| | | | TATACAAGGATAAATAAATTCTCACCTGC | |
| NM_001122 | ADFP | SEQ ID NO: 826 | GCTGCAGAAAATGAAGGAATCTTTAGATGAC | SEQ ID NO: 827 |
| | | | GTGATGGATTATCTTGTTAACAACACGCC | |
| NM_014294 | TRAM1 | SEQ ID NO: 828 | AGAAAATCAGAAACTGGATTTCAGTACTGGA | SEQ ID NO: 829 |
| | | | AACTTCAATGTGTTAGCTGTTAGAATCGC | |
| AK026912 | | SEQ ID NO: 830 | TACTTTCCCAATGCTTAGTAGCACTTGAGAC | SEQ ID NO: 831 |
| | | | TTTATTTCCATCGACAAAAACCAAGAAGG | |
| NM_005989 | AKR1D1 | SEQ ID NO: 832 | CTGTCATAGGCTGAGAGAGTTCTTCAAAAAT | SEQ ID NO: 833 |
| | | | TATGTTTTCCCAAGATCAGTTGCTTATAG | |

Table 7 shows the use of one of the genes, SCD, selected from Table 6 as an off-target signature gene to estimate the degree of off-target activity for a set of HSD1 inhibitor compounds. Microarray measurements were made as described in Example 2 for the HSD1 inhibitor compounds listed in Table 7. Each HSD1 inhibitor compound treated sample was compared to a forskolin alone cell sample. The HepG2/HSD1 cells used in these experiments were not contacted with cortisone. Table 7 lists the log ratio value measure for the microarray probe corresponding to the gene SCD.

TABLE 7
Estimation of HSD1 compound off-target activity
using SCD expression level.

| | Log ratio of |
|---|---|
| Compound Treatment | SCD gene expression |
| 10 μM Forskolin alone plus 3-(2-chlorophenyl)-4- | 0.45 |
| methyl-5-(4-pentylbicyclo[2.2.2]oct-1-yl)-4H- | |
| 1,2,4-triazole | |
| 10 μM Forskolin alone plus Compound 5 | 0.35 |
| 10 μM Forskolin alone plus Compound 2 | 0.32 |
| 10 μM Forskolin alone plus Compound 3 | 0.26 |
| 10 μM Forskolin alone plus Compound 6 | 0.23 |
| 10 μM Forskolin alone plus Compound 4 | 0.08 |
| 10 μM Forskolin alone plus Compound 1 | 0 |

The data in Table 7 show that measurement of SCD gene expression level is useful in the estimation of the off-target activity of an HSD1 inhibitor compound.

### Example 6

### Quantitative Real-time PCR Analysis of Cortisone Response Signature or Off-Target Response Signature Gene Activity in HepG2 Cells Expressing HSD1

HepG2 cells stably transfected with human HSD1, as previously described in Example 1, were plated in either 96 well or 24 well tissue culture plates. Cells were plated at a density of 6.25×10⁵ cells/well on 24 well plates in 500 μl DMEM culture medium prepared as previously described. For the 96 well plates, the cells were plated at a density of 1×10⁵ cells per well in 100 μl culture medium. The cells were incubated at 37° C. in a 5% CO₂ atmosphere. After 24 hours, the culture medium was removed and the cells were pre-incubated with, either 250 μl (24 well plate) or 50 μl (96 well plate), Culture Medium containing 2× concentration of HSD1 inhibitor (14 μM) or vehicle for 20 min at 37° C. in a 5% CO₂ atmosphere. Culture Medium (either 250 μl or 50 μl) containing 1000 nM cortisone (500 nM final concentration) or vehicle, and 20 μM forskolin (10 μM final concentration), was next added to the cells. The cells were further incubated for 20 hrs at 37° C. in a 5% CO₂ atmosphere. All conditions were run in duplicates.

HepG2/HSD1 cells were lysed using Trizol reagent (Life Technologies, Grand Island, N.Y.). Total RNA was isolated from HepG2/HSD1 lysate using the RNeasy 96-Kit (Qiagen Inc., Valencia, Calif.) following the manufacturer's instructions, including the optional on-column removal of contaminating DNA step by RNase-free DNase I treatment. The RNA was reverse-transcribed to single stranded cDNA using random hexamer primers with TAQman reverse transcription reagents (Applied Biosystems, Foster City, Calif.).

The sequences of the TAQman primers and probes used to detect the target cDNAs were synthesized by Qiagen Operon (Alameda, Calif.) and are shown in Tables 8 and 9.

TABLE 8
Real-time PCR primers and probes for cortisone response signature genes.

| Primer/ | | Reference | | Reference | | Primer/probe | |
|---|---|---|---|---|---|---|---|
| probe | Gene | transcript | SEQ ID NO: | Protein | SEQ ID NO: | Sequence | SEQ ID NO: |
| HuHOMER- | Homer-2 | NM_004839 | SEQ ID NO: 12 | NP_004830 | SEQ ID NO: 16 | CGCAGTTTGTCG | SEQ ID NO: 834 |
| 2_F | | NM_199330 | SEQ ID NO: 13 | NP_955362 | SEQ ID NO: 17 | TGTTTCCA | |
| | | NM_199331 | SEQ ID NO: 14 | NP_955363 | SEQ ID NO: 18 | | |
| | | NM_199332 | SEQ ID NO: 15 | NP_955364 | SEQ ID NO: 19 | | |
| HuHOMER- | Homer-2 | NM_004839 | SEQ ID NO: 12 | NP_004830 | SEQ ID NO: 16 | AAGCGAGAGGA | SEQ ID NO: 835 |
| 2_R | | NM_199330 | SEQ ID NO: 13 | NP_955362 | SEQ ID NO: 17 | GATTTCTATTCT | |
| | | NM_199331 | SEQ ID NO: 14 | NP_955363 | SEQ ID NO: 18 | GAA | |
| | | NM_199332 | SEQ ID NO: 15 | NP_955364 | SEQ ID NO: 19 | | |
| HuHOMER- | Homer-2 | NM_004839 | SEQ ID NO: 12 | NP_004830 | SEQ ID NO: 16 | ACCAGTTGTGC | SEQ ID NO: 836 |
| 2_Tqm | | NM_199330 | SEQ ID NO: 13 | NP_955362 | SEQ ID NO: 17 | CGTCCACTCAC | |
| (probe) | | NM_199331 | SEQ ID NO: 14 | NP_955363 | SEQ ID NO: 18 | | |
| | | NM_199332 | SEQ ID NO: 15 | NP_955364 | SEQ ID NO: 19 | | |
| hG6P-CAT-F | G6PC | NM_000151 | SEQ ID NO: 22 | NP_000142 | SEQ ID NO: 23 | TCCTCATCAAGT | SEQ ID NO: 837 |
| | | | | | | TGTTGCTGGA | |
| hG6P-CAT-R | G6PC | NM_000151 | SEQ ID NO: 22 | NP_000142 | SEQ ID NO: 23 | GCTGTGGATGT | SEQ ID NO: 838 |
| | | | | | | GGCTGAAAGT | |
| hG6P-CAT- | G6PC | NM_000151 | SEQ ID NO: 22 | NP_000142 | SEQ ID NO: 23 | TCCTGTCAGGC | SEQ ID NO: 839 |
| Tqm(probe) | | | | | | ATTGCTGTTGCA | |
| | | | | | | GA | |
| HuPGC_F | PGC | NM_002630 | SEQ ID NO: 20 | NP_002621 | SEQ ID NO: 21 | CCGGCTTCTTTG | SEQ ID NO: 840 |
| | | | | | | GCTATGAC | |
| HuPGC_R | PGC | NM_002630 | SEQ ID NO: 20 | NP_002621 | SEQ ID NO: 21 | ACTCAAGCCGA | SEQ ID NO: 841 |
| | | | | | | ACTCCTGGTT | |
| HuPGC_P | PGC | NM_002630 | SEQ ID NO: 20 | NP_002621 | SEQ ID NO: 21 | CTGACTGTCCA | SEQ ID NO: 842 |
| (probe) | | | | | | GAGCATCCAGG | |
| | | | | | | TC | |
| hTAT_F | TAT | NM_000353 | SEQ ID NO: 24 | NP_000344 | SEQ ID NO: 25 | TGTTCCTGCTTT | SEQ ID NO: 843 |
| | | | | | | TCCCCAAGT | |
| hTAT_R | TAT | NM_000353 | SEQ ID NO: 24 | NP_000344 | SEQ ID NO: 25 | AAGGGAGTAAT | SEQ ID NO: 844 |
| | | | | | | CTGCGATGTGA | |
| | | | | | | AT | |
| hTAT_P | TAT | NM_000353 | SEQ ID NO: 24 | NP_000344 | SEQ ID NO: 25 | CAACCACACCC | SEQ ID NO: 845 |
| (probe) | | | | | | ACACTCAGATC | |
| | | | | | | CTCCT | |
| HuLIPG_F | LIPG | NM_006033 | SEQ ID NO: 26 | NP_006024 | SEQ ID NO: 27 | CCCAGTGGAGT | SEQ ID NO: 846 |
| | | | | | | TGGGATTTCT | |
| HuLIPG_R | LIPG | NM_006033 | SEQ ID NO: 26 | NP_006024 | SEQ ID NO: 27 | GCCTTTCCCCAT | SEQ ID NO: 847 |
| | | | | | | GCATATACA | |
| HuLIPG_P | LIPG | NM_006033 | SEQ ID NO: 26 | NP_006024 | SEQ ID NO: 27 | CCTCTTTCTGTT | SEQ ID NO: 848 |
| (probe) | | | | | | TGGATGGTGTAT | |

TABLE 9
Real-time PCR primers and probes for off-target signature genes.

| | | Reference | | Reference | | Primer/Probe | |
|---|---|---|---|---|---|---|---|
| Primer/probe | Gene | transcript | SEQ ID NO: | Protein | SEQ ID NO: | Sequence | SEQ ID NO: |
| huDHCR7_F | DHCR7 | NM_001360 | SEQ ID NO: 28 | NP_001351 | SEQ ID NO: 29 | CACCTTTACTA | SEQ ID NO: 849 |
| | | | | | | GTCCTTTGGAG | |
| | | | | | | ACAA | |
| huDHCR7_R | DHCR7 | NM_001360 | SEQ ID NO: 28 | NP_001351 | SEQ ID NO: 29 | CACCAGTGTG | SEQ ID NO: 850 |
| | | | | | | GGCAGAGTGT | |
| huDHCR7_P | DHCR7 | NM_001360 | SEQ ID NO: 28 | NP_001351 | SEQ ID NO: 29 | TTACCTGTATT | SEQ ID NO: 851 |
| (probe) | | | | | | AGGAGCCCAG | |
| | | | | | | GCC | |
| HuSCD_F | SCD | NM_005063 | SEQ ID NO: 30 | NP_005054 | SEQ ID NO: 31 | CACCACATTCT | SEQ ID NO: 852 |
| | | | | | | TCATTGATTGCA | |
| HuSCD_R | SCD | NM_005063 | SEQ ID NO: 30 | NP_005054 | SEQ ID NO: 31 | GCGGCCTTGG | SEQ ID NO: 853 |
| | | | | | | AGACTTTCTT | |
| HuSCD_P | SCD | NM_005063 | SEQ ID NO: 30 | NP_005054 | SEQ ID NO: 31 | CCGCCCTCGGT | SEQ ID NO: 854 |
| (probe) | | | | | | CTGGCCTATG | |
| HuACLY_F | ACLY | NM_001096 | SEQ ID NO: 32 | NP_001087 | SEQ ID NO: 34 | GGTCACCGAG | SEQ ID NO: 855 |
| | | NM_198830 | SEQ ID NO: 33 | NP_942127 | SEQ ID NO: 35 | TGAAGTCGATA | |
| | | | | | | AA | |
| HuACLY_R | ACLY | NM_001096 | SEQ ID NO: 32 | NP_001087 | SEQ ID NO: 34 | GAAGTGCTGC | SEQ ID NO: 856 |
| | | NM_198830 | SEQ ID NO: 33 | NP_942127 | SEQ ID NO: 35 | CTGACGTAATC | |
| | | | | | | TT | |
| HuACLY_P | ACLY | NM_001096 | SEQ ID NO: 32 | NP_001087 | SEQ ID NO: 34 | AACCCAGACA | SEQ ID NO: 857 |
| (probe) | | NM_198830 | SEQ ID NO: 33 | NP_942127 | SEQ ID NO: 35 | TGCGAGTGCA | |
| | | | | | | GATC | |
| huFBXO9_F | FBXO9 | NM_012347 | SEQ ID NO: 36 | NP_036479 | SEQ ID NO: 39 | TCTTCCGATGG | SEQ ID NO: 858 |
| | | NM_033480 | SEQ ID NO: 37 | NP_258441 | SEQ ID NO: 40 | GTGGTGTCT | |
| | | NM_033481 | SEQ ID NO: 38 | NP_258442 | SEQ ID NO: 41 | | |
| huFBXO9_R | FBXO9 | NM_012347 | SEQ ID NO: 36 | NP_036479 | SEQ ID NO: 39 | TCTGCACACCA | SEQ ID NO: 859 |
| | | NM_033480 | SEQ ID NO: 37 | NP_258441 | SEQ ID NO: 40 | GCGACAACT | |
| | | NM_033481 | SEQ ID NO: 38 | NP_258442 | SEQ ID NO: 41 | | |
| huFBXO9_P | FBXO9 | NM_012347 | SEQ ID NO: 36 | NP_036479 | SEQ ID NO: 39 | TGACTTGGACC | SEQ ID NO: 860 |
| (probe) | | NM_033480 | SEQ ID NO: 37 | NP_258441 | SEQ ID NO: 40 | TCAGATCATTG | |
| | | NM_033481 | SEQ ID NO: 38 | NP_258442 | SEQ ID NO: 41 | GA | |
| huHEY1_F | HEY1 | NM_012258 | SEQ ID NO: 42 | NP_036390 | SEQ ID NO: 43 | GCTGCCTCTGC | SEQ ID NO: 861 |
| | | | | | | TCTCCTCAGT | |
| huHEY1_R | HEY1 | NM_012258 | SEQ ID NO: 42 | NP_036390 | SEQ ID NO: 43 | GTAAGTGGAA | SEQ ID NO: 862 |
| | | | | | | GGAGCCGAAA | |
| | | | | | | GA | |
| huHEY1_P | HEY1 | NM_012258 | SEQ ID NO: 42 | NP_036390 | SEQ ID NO: 43 | CCTCCCTGTCG | SEQ ID NO: 863 |
| (probe) | | | | | | GCCTTCCCCTT | |

Oligonucleotide probes were labeled with the 6-FAM fluorophore at the 5′ end (FAM) and a non-fluorescent quencher at the 3′ end (NFQ). Real-time PCR was performed on human HepG2/HSD1 cDNA using the TaqMan Universal PCR Master Mix (Applied Biosystems, Foster City, Calif.) and appropriate primer/probe sets on an ABI Prism 7900HT Sequence Detection System (Applied Biosystems, Foster City, Calif.) in the 384-well format according to manufacturer's instructions. The levels of mRNA were normalized to the amount of 18S RNA detected in each sample using the Eukaryotic 18S rRNA Endogenous Control from Applied Biosystems. Levels of the target mRNAs in treated cells were calculated relative to the untreated cells.

Table 10 shows the Taqman assay results for five cortisone signature genes obtained using cortisone and forskolin alone and in combination with HSD1 inhibitor compound 3-(2-chlorophenyl)-4-methyl-5-(4-pentylbicyclo[2.2.2]oct-1-yl)-4H-1,2,4-triazole and four other test agents.

TABLE 10
Cortisone signature genes measured by RT-PCR.

| | Gene Transcript Measured | | | | | |
|---|---|---|---|---|---|---|
| | (Fold Change Relative to Vehicle Alone) | | | | | |
| | | | | | | Average |
| | | | | | | of five |
| Compound Treatment | Homer-2 | G6PC | PGC | TAT | LIPG | genes |
| Cortisone/Forskolin (CF) | 18 | 2.8 | 27 | 5.8 | 2.9 | 11.3 |
| CF plus Compound 7 [404′] | 14.7 | 2.4 | 28 | 6.8 | 3.3 | 11.0 |
| CF plus Compound 8 [851′] | 5.8 | 2.2 | 8.0 | 4.2 | 5.4 | 5.1 |
| CF plus Compound 9 [051′] | 2.0 | 1.6 | 2.5 | 3.0 | 2.2 | 2.7 |
| CF plus Compound 10 [400′] | 1.1 | 0.8 | 0.8 | 1.2 | 1.4 | 1.1 |
| CF plus 3-(2-chlorophenyl)-4- | 1.5 | 1.0 | 0.7 | 1.2 | 2.7 | 1.4 |
| methyl-5-(4- | | | | | | |
| pentylbicyclo[2.2.2]oct-1-yl)- | | | | | | |
| 4H-1,2,4-triazole | | | | | | |
| CF plus Compound 11 [750′] | 1.1 | 1.2 | 0.5 | 1.0 | 1.0 | 0.96 |

The results in Table 10 show that the various HSD1 inhibitors can be compared to each other based upon the changes in gene expression caused by each compound as compared to the Cortisone/Forskolin treated cells alone either on a gene by gene basis or by averaging across all five genes. For example, based upon these results it is apparent that Compound 7 is not as strong of an HSD1 inhibitor as compared to the other compounds tested in this experiment because it does not strongly reverse the gene expression levels measured for the cortisone/forskolin alone treatment. In contrast, Compounds 10 and 11 are strong HSD1 inhibitor agents as is reflected in the almost total reversal in the expression levels measured for these five cortisone signature genes as compared to the cortisone/forskolin alone treatment.

Table 11 shows the Taqman assay results for five off-target signature genes obtained using forskolin treatment alone and in combination with HSD1 inhibitor compound 3-(2-chlorophenyl)-4-methyl-5-(4-pentylbicyclo[2.2.2]oct-1-yl)-4H-1,2,4-triazole and four other test HSD1 inhibitor compounds.

TABLE 11
Off-target signature genes measured by RT-PCR.

| | Gene Transcript Measured | | | | | |
|---|---|---|---|---|---|---|
| | (Fold Change Relative to Vehicle Alone) | | | | | |
| | | | | | | Average |
| | | | | | | of five |
| Compound Treatment | SCD | DHCR7 | ACLY | FBXO9 | HEY1 | genes |
| Forskolin plus 3-(2- | 2.3 | 2.2 | 2.2 | 0.9 | 0.9 | 1.7 |
| chlorophenyl)-4-methyl-5-(4- | | | | | | |
| pentylbicyclo[2.2.2] oct-1-yl)- | | | | | | |
| 4H-1,2,4-triazole | | | | | | |
| CF plus Compound 8 [851′] | 1.8 | 2.6 | 2.7 | 0.8 | 0.7 | 1.7 |
| CF plus Compound 9 [051′] | 1.2 | 1.2 | 0.9 | 1.6 | 1.2 | 1.5 |
| CF plus Compound 11 [750′] | 1.3 | 1.4 | 1.0 | 2.3 | 1.3 | 1.46 |
| Forskolin plus Compound 7 | 1.3 | 1.5 | 1.1 | 1.2 | 1.0 | 1.2 |
| [404′] | | | | | | |
| CF plus Compound 10 [400′] | 1.1 | 1.1 | 0.9 | 1.3 | 0.8 | 1.0 |

The results in Table 11 show that the various HSD1 inhibitors can be compared to each other and rank ordered based upon the changes in gene expression caused by each compound as compared to the cells treated with an HSD1 inhibitor known to induce changes in gene expression of many off-target gene, e.g., 3-(2-chlorophenyl)-4-methyl-5-(4-pentylbicyclo[2.2.2]oct-1-yl)-4H-1,2,4-triazole. The HSD1 inhibitor compounds may be compared using each single gene measurement, i.e., on a gene by gene basis, or by averaging the fold change in expression levels across all five off-target genes. For example, based upon these results it is apparent that Compound 8 has a high level of off-target effects as compared to Compounds 7 and 10. However, as is apparent from the data in Table 10, Compound 7 is a poor inhibitor of HSD1, while Compound 10 has high inhibitor activity, but low off-target effects.

### REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method of identifying an agent that modifies HSD1 activity comprising
a) measuring expression levels of one or more genes in a test sample from a HepG2 hepatoma cell line contacted with an agent and cortisone, wherein said HepG2 hepatoma cell line expresses HSD1 at a level that is detectably higher than the level of HSD1 in unmodified HepG2 hepatoma cells, and each of said one or more genes corresponds to a marker listed in Table 2; and
b) comparing said measured gene expression levels of said one or more genes in said test sample to gene expression levels of said one or more genes measured in a control sample, wherein said control sample is obtained from said HepG2 hepatoma cell line that expresses HSD1, is contacted with cortisone, and is not contacted with said test agent,
wherein a difference in said expression levels of said one or more genes in said test sample as compared to said control sample indicates that said test agent modifies HSD1 activity.

2. The method of claim 1 wherein said plurality of genes comprises three or more protein sequences of markers listed in Table 2.

3. The method of claim 1 wherein said measuring of expression level of one or more genes is determined using a method selected from the group consisting of a microarray, RT-PCR, Northern blot, and detecting protein level.

4. The method of claim 1 wherein said one or more genes comprises three or more nucleotide sequences selected from Table 2.

5. The method of claim 1 wherein said one or more genes comprises five or more genes selected from Table 2.

6. The method of claim 1 wherein said one or more genes comprises ten or more genes selected from Table 2.

7. A method of estimating HSD1 off-target activity of an agent comprising:
a) contacting an agent that modifies HSD1 activity with a first sample of HepG2 hepatoma cell line cells, wherein said cell line over-expresses HSD1;
b) measuring an expression level of one or more genes in said first sample, wherein said one or more genes are indicative of HSD1 off-target activity;
c) comparing each said measured expression level to a second expression level of each of said one or more genes in a second sample of cells from said HepG2 hepatoma cell line not contacted with said agent;
wherein a change in expression level of said one or more genes between said first sample and said second sample provides an estimate of HSD1 off-target activity.

8. The method of claim 7 wherein said measuring an expression level is determined using a method selected from the group consisting of a microarray, RT-PCR, Northern blot, and detecting protein level.

9. The method of claim 7 wherein said one or more genes comprise SCD1, DHCR7, ACLY, FBXO9 or HEY1.

10. The method of claim 7 wherein said expression level is measured for a plurality of genes indicative of HSD1 off-target activity.

11. The method of claim 10 wherein said plurality of genes comprises five or more genes, each gene in said five or more genes corresponding to a sequence selected from Table 6

12. The method of claim 11 wherein said five or more genes comprise SCD1, DHCR7, ACLY, FBXO9 and HEY1.

13. The method of claim 7 wherein said measuring an expression level is determined by measuring the transcript level of said one or more genes.

14. The method of claim 7 wherein said one or more genes comprises SCD1.

15. The method of claim 7, wherein HSD1 is expressed in said HepG2 hepatoma cell line at least two-fold higher than expression of HSD1 in a HepG2 hepatoma cell line that does not over-express HSD1.

16. The method of claim 7, wherein HSD1 is expressed in said HepG2 hepatoma cell line at least five-fold higher than expression of HSD1 in a HepG2 hepatoma cell line that does not over-express HSD1.

17. The method of claim 7, wherein HSD1 is expressed in said HepG2 cell line at least ten-fold higher than expression of HSD1 in a HepG2 hepatoma cell line that does not over-express HSD1.

18. The method of claim 7, wherein steps a) and b) are repeated using a second agent and wherein said first and second agents are rank ordered based on the magnitude of change in expression level of said one or more genes between said first samples contacted with said first and second agents compared to said second cell sample not contacted with said agents.

19. The method of claim 7, wherein steps a) and b) are performed for each of a plurality of additional agents and wherein said agents are rank ordered based on the magnitude of change in expression level of said one or more genes between said first samples contacted with said plurality of agents compared to said second cell sample not contacted with said plurality of agents, and wherein the agent exhibiting the largest change in gene expression between said first sample and second sample compared to the other agents within said plurality of agents is identified at having the highest level of off-target HSD1 activity.

20. A method of sorting HSD1-agents comprising:
a) contacting a subject with an HSD1-agent and cortisone, wherein said subject has detectable HSD1 activity;
b) measuring expression levels of a plurality of genes in a test sample obtained from said subject after contact with said HSD1-agent and cortisone, wherein each of said plurality of genes corresponds to the markers listed in Table 2, and
c) comparing said measured gene expression levels in said test sample to gene expression levels for said plurality of genes measured in a control sample to obtain a gene expression difference result,
wherein said control sample is obtained from said subject and contacted with cortisone and not contacted with said HSD1-agent,
wherein said HSD1-agent is sorted by the magnitude of said gene expression difference result.
